SEQUENCE LISTING (10 sequences)

SEQ ID NO: 1 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 4 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 5 - protein, 5 aa, synthetic construct
SYGMH

SEQ ID NO: 6 - protein, 17 aa, synthetic construct
IIWFDGSSTYYADSVRG

SEQ ID NO: 7 - protein, 9 aa, synthetic construct
ELGRRYFDL

SEQ ID NO: 8 - protein, 11 aa, synthetic construct
RASQSVSSYLA

SEQ ID NO: 9 - protein, 7 aa, synthetic construct
DASKRAT

SEQ ID NO: 10 - protein, 11 aa, synthetic construct
QQRSKWPPWTF

(19) United States
(12) Patent Application Publication
(10) Pub. No.: US 2024/0043545 A1
(43) Pub. Date: Feb. 8, 2024

(54) METHODS AND COMPOSITIONS FOR TREATMENT OF THYROID EYE DISEASE

(71) Applicant: Horizon Therapeutics Ireland DAC, Dublin 2 (IE)

(72) Inventors: Saba SILE, Oakland, CA (US); Jeffrey Wayne SHERMAN, Lincolnshire, IL (US); Robert John HOLT, Vernon Hills, IL (US); Theresa Anne PODREBARAC, Millis, MA (US); Shayna Ravindran MELVANI, Oak Brook, IL (US)

(21) Appl. No.: 18/324,056

(22) Filed: May 25, 2023

Related U.S. Application Data
(60) Provisional application No. 63/386,528, filed on Dec. 8, 2022.
(60) Provisional application No. 63/346,069, filed on May 26, 2022.

(51) Int. Cl. C07K 16/28; A61K 45/06; A61K 39/395; A61P 27/02

(52) U.S. Cl. CPC C07K 16/2863; A61K 45/06; A61K 39/3955; A61P 27/02

(57) ABSTRACT

Provided herein are methods and compositions for treating an individual afflicted with an insulin-like growth factor 1 receptor (IGF1R) associated disorder using an antagonist of IGF1R signaling.

### CROSS-REFERENCE

This application claims the benefit of U.S. Provisional Application No. 63/346,069, filed May 26, 2022, and U.S. Provisional Application No. 63/386,528, filed Dec. 8, 2022, both of which are herein entirely incorporated by reference.

### SEQUENCE LISTING

The instant application contains a Sequence Listing which has been submitted electronically in XML format and is hereby incorporated by reference in its entirety. Said XML file, created on Oct. 5, 2023, is named “58651-821_201_SL.xml” and is 12,288 bytes in size.

### BACKGROUND

The insulin-like growth factor 1 receptor (IGF1R) is a cell surface receptor. The IGF1R signaling is involved in a number of disorders, such as thyroid eye disease (TED), also known as Graves' eye disease, Graves' ophthalmopathy, Graves' orbitopathy, thyroid-associated ophthalmopathy (TAO). The thyroid eye disease may affect the thyroid and eyes, and cause inflammation and damage to the muscle and connective tissues of the eyes, leading to pain, swelling, disfigurement of the eye socket, poor vision, or blindness.

There exists a need for methods and composition for the treatment of IGF1R associated disorders, such as thyroid eye disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

In an aspect, provided herein is a method of treating an individual afflicted with an insulin-like growth factor 1 receptor (IGF1R) associated disorder, the method comprising: a) selecting an individual afflicted with the IGF1R associated disorder for treatment with an antagonist of IGF1R signaling if the individual exhibits normal blood glucose control by a test for blood glucose control; and b) administering the antagonist of IGF1R signaling to the individual. In some embodiments, the IGF1R associated disorder comprises thyroid eye disease. In some embodiments, the antagonist of IGF1R signaling comprises an antibody that binds IGF1R. In some embodiments, the antibody that binds IGF1R comprises ganitumab, figitumumab, MEDI-573, cixutumumab, dalotuzumab, robatumumab, AVE1642, BIIB022, xentuzumab, istiratumab, or linsitinib. In some embodiments, the antibody that binds IGF1R comprises teprotumumab. In some embodiments, the antagonist of IGF1R signaling comprises a small molecule that inhibits IGF1R signaling. In some embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, or A-928605. In some embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In some embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In some embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In some embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In some embodiments, the antibody that binds IGF1R comprises: a) a heavy chain comprising an amino acid sequence according to SEQ ID No.: 1; and b) a light chain comprising an amino acid sequence according to SEQ ID No.: 2.

In some embodiments, the test for blood glucose control comprises a measurement of hemoglobin A1c (HbA1c), an oral glucose tolerance test, a measurement of fasting blood glucose, a measurement of non-fasting blood glucose, or any combination thereof. In some embodiments, the test for blood glucose control comprises a measurement of hemoglobin A1c (HbA1c). In some embodiments, the individual exhibits normal blood glucose control if the measurement of hemoglobin A1c (HbA1c) is below about 6.5%. In some embodiments, the individual exhibits normal blood glucose control if the measurement of hemoglobin A1c (HbA1c) is below about 5.7%. In some embodiments, the test for blood glucose control comprises an oral glucose tolerance test. In some embodiments, the individual exhibits normal blood glucose control if the individual exhibits a blood glucose level below about 200 mg/dL about two hours after oral ingestion of about 75 grams of sugar. In some embodiments, the individual exhibits normal blood glucose control if the individual exhibits a blood glucose level of about 140 mg/dL or less about two hours after oral ingestion of about 75 grams of sugar. In some embodiments, the test for blood glucose control comprises a measurement of fasting blood glucose. In some embodiments, the individual exhibits normal blood glucose control if the individual exhibits a blood glucose level below about 126 mg/dL after an overnight fast. In some embodiments, the individual exhibits normal blood glucose control if the individual exhibits a blood glucose level of about 99 mg/dL or less after an overnight fast. In some embodiments, the overnight fast lasts at least about 8 hours. In some embodiments, the overnight fast lasts between about 8 and about 16 hours. In some embodiments, the test for blood glucose control comprises a measurement of non-fasting blood glucose. In some embodiments, the individual exhibits normal blood glucose control if the individual exhibits a non-fasting blood glucose level below about 200 mg/dL. In some embodiments, the individual is diagnosed with hyperglycemia, prediabetes, or diabetes before the treatment with the antagonist of IGF1R signaling. In some embodiments, the administering comprises intravenous infusion. In some embodiments, the administering comprises administering about 10 mg/kg of the antagonist of IGF1R signaling over the course of about 60 min to about 90 min based on the weight of the individual. In some embodiments, the administering further comprises administering about 20 mg/kg of the antagonist of IGF1R signaling over the course of about 60 min to about 90 min based on the weight of the individual. In some embodiments, the administering further comprises administering about 20 mg/kg of the antagonist of IGF1R signaling once every 3 weeks for about 7 times.

In another aspect, provided herein is a method of treating an individual afflicted with an insulin-like growth factor 1 receptor (IGF1R) associated disorder the method comprising: a) obtaining the results of a test for blood glucose control from the individual; and b) administering an antagonist of IGF1R signaling to the individual if the individual exhibits normal blood glucose control by the test for blood glucose control. In some embodiments, the IGF1R associated disorder comprises thyroid eye disease. In some embodiments, the antagonist of IGF1R signaling comprises an antibody that binds IGF1R. In some embodiments, the antibody that binds IGF1R comprises ganitumab, figitumumab, MEDI-573, cixutumumab, dalotuzumab, robatumumab, AVE1642, BIIB022, xentuzumab, istiratumab, or linsitinib. In some embodiments, the antibody that binds IGF1R comprises teprotumumab. In some embodiments, the antagonist of IGF1R signaling comprises a small molecule that inhibits IGF1R signaling. In some embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, or A-928605. In some embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In some embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In some embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In some embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In some embodiments, the antibody that binds IGF1R comprises: a) a heavy chain comprising an amino acid sequence according to SEQ ID No.: 1; and b) a light chain comprising an amino acid sequence according to SEQ ID No.: 2.

In some embodiments, the test for blood glucose control comprises a measurement of hemoglobin A1c (HbA1c), an oral glucose tolerance test, a measurement of fasting blood glucose, a measurement of non-fasting blood glucose, or any combination thereof. In some embodiments, the test for blood glucose control comprises a measurement of hemoglobin A1c (HbA1c). In some embodiments, the individual exhibits normal blood glucose control if the measurement of HbA1c is below about 6.5%. In some embodiments, the individual exhibits normal blood glucose control if the measurement of HbA1c is below about 5.7%. In some embodiments, the test for blood glucose control comprises an oral glucose tolerance test. In some embodiments, the individual exhibits normal blood glucose control if the individual exhibits a blood glucose level below about 200 mg/dL about two hours after oral ingestion of about 75 grams of sugar. In some embodiments, the individual exhibits normal blood glucose control if the individual exhibits a blood glucose level of about 140 mg/dL or less about two hours after oral ingestion of about 75 grams of sugar. In some embodiments, the test for blood glucose control comprises a measurement of fasting blood glucose. In some embodiments, the individual exhibits normal blood glucose control if the individual exhibits a blood glucose level below about 126 mg/dL after an overnight fast. In some embodiments, the individual exhibits normal blood glucose control if the individual exhibits a blood glucose level of about 99 mg/dL or less after an overnight fast. In some embodiments, the overnight fast lasts at least about 8 hours. In some embodiments, the overnight fast lasts between about 8 and about 16 hours. In some embodiments, the test for blood glucose control comprises a measurement of non-fasting blood glucose. In some embodiments, the individual exhibits normal blood glucose control if the individual exhibits a non-fasting blood glucose level below about 200 mg/dL. In some embodiments, the individual is diagnosed with hyperglycemia, prediabetes, or diabetes before the treatment with the antagonist of IGF1R signaling. In some embodiments, the administering comprises intravenous infusion. In some embodiments, the administering comprises administering about 10 mg/kg of the antagonist of IGF1R signaling over the course of about 60 min to about 90 min based on the weight of the individual. In some embodiments, the administering further comprises administering about 20 mg/kg of the antagonist of IGF1R signaling over the course of about 60 min to about 90 min based on the weight of the individual. In some embodiments, the administering further comprises administering about 20 mg/kg of the antagonist of IGF1R signaling once every 3 weeks for about 7 times.

In another aspect, provided herein is a method of monitoring an individual being treated with an antagonist of insulin-like growth factor 1 receptor (IGF1R) signaling, wherein the individual is afflicted with an IGF1R associated disorder, the method comprising: a) obtaining the results of a test for blood glucose control from the individual being treated with the antagonist of IGF1R signaling; and b) interrupting the treatment of the individual with the antagonist of IGF1R signaling if the individual exhibits abnormal blood glucose control by the test for blood glucose control. In some embodiments, the IGF1R associated disorder comprises thyroid eye disease. In some embodiments, the antagonist of IGF1R signaling comprises an antibody that binds IGF1R. In some embodiments, the antibody that binds IGF1R comprises ganitumab, figitumumab, MEDI-573, cixutumumab, dalotuzumab, robatumumab, AVE1642, BIIB022, xentuzumab, istiratumab, or linsitinib. In some embodiments, the antibody that binds IGF1R comprises teprotumumab. In some embodiments, the antagonist of IGF1R signaling comprises a small molecule that inhibits IGF1R signaling. In some embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, or A-928605. In some embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In some embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In some embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In some embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In some embodiments, the antibody that binds IGF1R comprises: a) a heavy chain comprising an amino acid sequence according to SEQ ID No.: 1; and b) a light chain comprising an amino acid sequence according to SEQ ID No.: 2.

In some embodiments, the test for blood glucose control comprises a measurement of hemoglobin A1c (HbA1c), an oral glucose tolerance test, a measurement of fasting blood glucose, a measurement of non-fasting blood glucose, or any combination thereof. In some embodiments, the test for blood glucose control comprises a measurement of hemoglobin A1c (HbA1c). In some embodiments, the individual exhibits abnormal blood glucose control if the measurement of HbA1c is about 6.5% or above. In some embodiments, the individual exhibits abnormal blood glucose control if the measurement of HbA1c is above about 5.7% or above. In some embodiments, the test for blood glucose control comprises an oral glucose tolerance test. In some embodiments, the individual exhibits abnormal blood glucose control if the individual exhibits a blood glucose level of at least about 200 mg/dL about two hours after oral ingestion of about 75 grams of sugar. In some embodiments, the individual exhibits abnormal blood glucose control if the individual exhibits a blood glucose level above about 140 mg/dL about two hours after oral ingestion of about 75 grams of sugar. In some embodiments, the test for blood glucose control comprises a measurement of fasting blood glucose. In some embodiments, the individual exhibits abnormal blood glucose control if the measurement of fasting blood glucose is about 126 mg/dL or above after an overnight fast. In some embodiments, the individual exhibits abnormal blood glucose control if the measurement of fasting blood glucose is above about 99 mg/dL after an overnight fast. In some embodiments, the overnight fast lasts at least about 8 hours. In some embodiments, the overnight fast lasts between about 8 and about 16 hours. In some embodiments, the test for blood glucose control comprises a measurement of non-fasting blood glucose. In some embodiments, the individual exhibits abnormal blood glucose control if the individual exhibits a non-fasting blood glucose level of about 200 mg/dL or above. In some embodiments, the individual is diagnosed with pre-existing hyperglycemia, prediabetes, or diabetes before the treatment with the antagonist of IGF1R signaling. In some embodiments, the administering comprises intravenous infusion. In some embodiments, the method further comprises obtaining the results of a second test for blood glucose control from the individual after treating the individual for abnormal blood glucose control. In some embodiments, the method further comprises administering the antagonist of IGF1R signaling to the individual if the individual exhibits normal blood glucose control by the second test for blood glucose control. In some embodiments, the administering further comprises administering about 10 mg/kg of the antagonist of IGF1R signaling based on the weight of the individual. In some embodiments, the administering further comprises administering about 20 mg/kg of the antagonist of IGF1R signaling based on the weight of the individual once every 3 weeks for about 7 times.

In another aspect, provided herein is a method of treating an individual afflicted with an insulin-like growth factor 1 receptor (IGF1R) associated disorder, the method comprising: a) administering a first dose of an antagonist of IGF1R signaling to the individual; b) obtaining the results of a test for blood glucose control from the individual; and c) administering a second dose of the antagonist of IGF1R signaling to the individual if the individual exhibits abnormal blood glucose control by the test for blood glucose control, wherein the second dose is lower than the first dose. In some embodiments, the IGF1R associated disorder comprises thyroid-eye disease. In some embodiments, the antagonist of IGF1R signaling comprises an antibody that binds IGF1R. In some embodiments, the antibody that binds IGF1R comprises ganitumab, figitumumab, MEDI-573, cixutumumab, dalotuzumab, robatumumab, AVE1642, BI113022, xentuzumab, istiratumab, or linsitinib. In some embodiments, the antibody that binds IGF1R comprises teprotumumab. In some embodiments, the antagonist of IGF1R signaling comprises a small molecule that inhibits IGF1R signaling. In some embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, or A-928605. In some embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In some embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In some embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In some embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In some embodiments, the antibody that binds IGF1R comprises: a) a heavy chain comprising an amino acid sequence according to SEQ ID No.: 1; and b) a light chain comprising an amino acid sequence according to SEQ ID No.: 2.

In some embodiments, the test for blood glucose control comprises a measurement of hemoglobin A1c (HbA1c), an oral glucose tolerance test, a measurement of fasting blood glucose, a measurement of non-fasting blood glucose, or any combination thereof. In some embodiments, the test for blood glucose control comprises a measurement of hemoglobin A1c (HbA1c). In some embodiments, the individual exhibits abnormal blood glucose control if the measurement of HbA1c is about 6.5% or above. In some embodiments, the individual exhibits abnormal blood glucose control if the measurement of HbA1c is about 5.7% or above. In some embodiments, the test for blood glucose control comprises an oral glucose tolerance test. In some embodiments, the individual exhibits abnormal blood glucose control if the individual exhibits a blood glucose level of about 200 mg/dL or above about two hours after oral ingestion of about 75 grams of sugar. In some embodiments, the individual exhibits abnormal blood glucose control if the individual exhibits a blood glucose level above about 140 mg/dL about two hours after oral ingestion of about 75 grams of sugar. In some embodiments, the test for blood glucose control comprises a measurement of fasting blood glucose. In some embodiments, the individual exhibits abnormal blood glucose control if the measurement of fasting blood glucose is about 126 mg/dL or above after an overnight fast. In some embodiments, the individual exhibits abnormal blood glucose control if the measurement of fasting blood glucose is above about 99 mg/dL after an overnight fast. In some embodiments, the overnight fast lasts at least about 8 hours. In some embodiments, the overnight fast lasts between about 8 and about 16 hours. In some embodiments, the test for blood glucose control comprises a measurement of non-fasting blood glucose. In some embodiments, the individual exhibits abnormal blood glucose control if the individual exhibits a non-fasting blood glucose level of about 200 mg/dL or above. In some embodiments, the individual is diagnosed with pre-existing hyperglycemia, prediabetes or diabetes before the treatment with the antagonist of IGF1R signaling. In some embodiments, the administering comprises intravenous infusion. In some embodiments, the first dose comprises about 10 mg/kg of the antagonist of IGF1R signaling being administered over the course of about 60 min to about 90 min based on the weight of the individual. In some embodiments, the first dose further comprises about 20 mg/kg of the antagonist of IGF1R signaling being administered over the course of about 60 min to about 90 min based on the weight of the individual. In some embodiments, the about 20 mg/kg of the antagonist of IGF1R signaling is administered to the individual once every 3 weeks for about 7 times.

In another aspect, provided herein is a method of treating an individual afflicted with an insulin-like growth factor 1 receptor (IGF1R) associated disorder, the method comprising: a) selecting an individual afflicted with the IGF1R associated disorder for treatment with an antagonist of IGF1R signaling if the individual exhibits normal blood glucose control by a first test for blood glucose control; b) administering a first dose of the antagonist of IGF1R signaling to the individual; and c) obtaining the results of a second test for blood glucose control from the individual being treated with the antagonist of IGF1R signaling. In some embodiments, the method further comprises interrupting treatment of the individual with the antagonist of IGF1R signaling if the individual exhibits abnormal blood glucose control by the results of the second test for blood glucose control. In some embodiments, the method further comprises administering a second dose of the antagonist of IGF1R signaling to the individual if the individual exhibits abnormal blood glucose control by the results of the second test for blood glucose control. In some embodiments, the IGF1R associated disorder comprises thyroid-eye disease. In some embodiments, the antagonist of IGF1R signaling comprises an antibody that binds IGF1R. In some embodiments, the antibody that binds IGF1R comprises ganitumab, figitumumab, MEDI-573, cixutumumab, dalotuzumab, robatumumab, AVE1642, BI113022, xentuzumab, istiratumab, or linsitinib. In some embodiments, the antibody that binds IGF1R comprises teprotumumab. In some embodiments, the antagonist of IGF1R signaling comprises a small molecule that inhibits IGF1R signaling. In some embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, or A-928605. In some embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In some embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In some embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In some embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In some embodiments, the antibody that binds IGF1R comprises: a) a heavy chain comprising an amino acid sequence according to SEQ ID No.: 1; and b) a light chain comprising an amino acid sequence according to SEQ ID No.: 2.

In some embodiments, the first or second test for blood glucose control comprises a measurement of hemoglobin A1c (HbA1c), an oral glucose tolerance test, a measurement of fasting blood glucose, a measurement of non-fasting blood glucose, or any combination thereof. In some embodiments, the first or second test for blood glucose control comprises a measurement of hemoglobin A1c (HbA1c). In some embodiments, the individual exhibits abnormal blood glucose control if the measurement of HbA1c is about 6.5% or above, wherein the individual exhibits normal blood glucose control if the measurement of hemoglobin A1c (HbA1c) is below about 6.5%. In some embodiments, the individual exhibits abnormal blood glucose control if the measurement of HbA1c is about 5.7% or above, wherein the individual exhibits normal blood glucose control if the measurement of hemoglobin A1c (HbA1c) is below about 5.7%. In some embodiments, the test for blood glucose control comprises an oral glucose tolerance test. In some embodiments, the individual exhibits abnormal blood glucose control if the individual exhibits a blood glucose level of at least about 200 mg/dL about two hours after oral ingestion of about 75 grams of sugar, wherein the individual exhibits normal blood glucose control if the individual exhibits a blood glucose level below about 200 mg/dL about two hours after oral ingestion of about 75 grams of sugar. In some embodiments, the individual exhibits abnormal blood glucose control if the individual exhibits a blood glucose level above about 140 mg/dL about two hours after oral ingestion of 75 grams of sugar, wherein the individual exhibits normal blood glucose control if the individual exhibits a blood glucose level of about 140 mg/dL or less about two hours after oral ingestion of about 75 grams of sugar. In some embodiments, the test for blood glucose control comprises a measurement of fasting blood glucose. In some embodiments, the individual exhibits abnormal blood glucose control if the measurement of fasting blood glucose is about 126 mg/dL or above after an overnight fast, wherein the individual exhibits normal blood glucose control if the individual exhibits a blood glucose level below about 126 mg/dL after an overnight fast. In some embodiments, the individual exhibits abnormal blood glucose control if the measurement of fasting blood glucose is above about 99 mg/dL after an overnight fast, wherein the individual exhibits normal blood glucose control if the individual exhibits a blood glucose level of about 99 mg/dL or less after an overnight fast. In some embodiments, the overnight fast lasts at least about 8 hours. In some embodiments, the overnight fast lasts between about 8 and about 16 hours. In some embodiments, the test for blood glucose control comprises a measurement of non-fasting blood glucose. In some embodiments, the individual exhibits abnormal blood glucose control if the individual exhibits a non-fasting blood glucose level of about 200 mg/dL or above, wherein the individual exhibits normal blood glucose control if the individual exhibits a non-fasting blood glucose level below about 200 mg/dL. In some embodiments, the individual is diagnosed with pre-existing hyperglycemia, prediabetes, or diabetes before the treatment with the antagonist of IGF1R signaling. In some embodiments, the administering comprises intravenous infusion. In some embodiments, the first dose comprises about 10 mg/kg of the antagonist of IGF1R signaling being administered over the course of about 60 min to about 90 min based on the weight of the individual. In some embodiments, the first dose further comprises about 20 mg/kg of the antagonist of IGF1R signaling being administered over the course of about 60 min to about 90 min based on the weight of the individual. In some embodiments, the about 20 mg/kg of the antagonist of IGF1R signaling is administered to the individual once every 3 weeks for 7 times. In some embodiments, the method further comprises treating the individual for abnormal blood control if the individual exhibits abnormal blood glucose control by the results of the second test for blood glucose control.

Described herein in one aspect is a method of treating an individual afflicted with an insulin-like growth factor 1 receptor (IGF1R) associated disorder, the method comprising: a) obtaining the results of a test for blood glucose from the individual; and b) administering a dose of the antagonist of IGF1R signaling to the individual if the individual does not exhibit elevated blood glucose by the test for blood glucose. In certain embodiments, the IGF1R associated disorder comprises thyroid eye disease. In certain embodiments, the antagonist of IGF1R signaling comprises an antibody that binds IGF1R. In certain embodiments, the antibody that binds IGF1R comprises ganitumab, figitumumab, MEDI-573, cixutumumab, dalotuzumab, robatumumab, AVE1642, BIIB022, xentuzumab, istiratumab, or linsitinib. In certain embodiments, the antibody that binds IGF1R comprises teprotumumab. In certain embodiments, the antagonist of IGF1R signaling comprises a small molecule that inhibits IGF1R signaling. In certain embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, or A-928605. In certain embodiments, the antibody that binds IGF1R comprises: a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In certain embodiments, the antibody that binds IGF1R comprises: a) a heavy chain variable domain comprising an amino acid sequence having at least 85%, 90%, or 95% sequence identity to SEQ ID No.: 3; and b) a light chain variable domain comprising an amino acid sequence having at least 85%, 90%, or 95% sequence identity to SEQ ID No.: 4. In certain embodiments, the antibody that binds IGF1R comprises: a) a heavy chain variable domain comprising an amino acid sequence according to SEQ ID No.: 3; and b) a light chain variable domain comprising an amino acid sequence according to SEQ ID No.: 4. In certain embodiments, the antibody that binds IGF1R comprises: a) a heavy chain comprising an amino acid sequence having at least 85%, 90%, or 95% sequence identity to SEQ ID No.: 1; and b) a light chain comprising an amino acid sequence having at least 85%, 90%, or 95% sequence identity to SEQ ID No.: 2. In certain embodiments, the antibody that binds IGF1R comprises: a) a heavy chain comprising an amino acid sequence according to SEQ ID No.: 1; and b) a light chain comprising an amino acid sequence according to SEQ ID No.: 2. In certain embodiments, the test for blood glucose comprises a measurement of hemoglobin A1c (HbA1c), an oral glucose tolerance test, a measurement of fasting blood glucose, a measurement of non-fasting blood glucose, or any combination thereof. In certain embodiments, the test for blood glucose comprises a measurement of hemoglobin A1c (HbA1c). In certain embodiments, the individual does not exhibit elevated blood glucose if the measurement of HbA1c is about 6.5% or below. In certain embodiments, the individual does not exhibit elevated blood glucose if the measurement of HbA1c is about 5.7% or below. In certain embodiments, the test for blood glucose comprises an oral glucose tolerance test. In certain embodiments, the individual does not exhibit elevated blood glucose if the individual exhibits a blood glucose level below 200 mg/dL after about two hours after oral ingestion of about 75 grams of sugar. In certain embodiments, the individual does not exhibit elevated blood glucose if the individual exhibits a blood glucose level below 140 mg/dL after about two hours after oral ingestion of about 75 grams of sugar. In certain embodiments, the test for blood glucose comprises a measurement of fasting blood glucose. In certain embodiments, the individual does not exhibit elevated blood glucose if the measurement of fasting blood glucose is below 126 mg/dL after an overnight fast. In certain embodiments, the individual does not exhibit elevated blood glucose if the measurement of fasting blood glucose is below 99 mg/dL after an overnight fast. In certain embodiments, the overnight fast lasts at least about 8 hours. In certain embodiments, the overnight fast lasts between about 8 and about 16 hours. In certain embodiments, the test for blood glucose comprises a measurement of non-fasting blood glucose. In certain embodiments, the individual does not exhibit elevated blood glucose if the individual exhibits a non-fasting blood glucose level of below about 200 mg/dL. In certain embodiments, the individual is diagnosed with pre-existing hyperglycemia, prediabetes or diabetes before the treatment with the antagonist of IGF1R signaling. In certain embodiments, the administering comprises intravenous infusion. In certain embodiments, dose comprises about 10 mg/kg of the antagonist of IGF1R signaling being administered over the course of about 60 min to about 90 min based on the weight of the individual. In certain embodiments, the dose comprises about 20 mg/kg of the antagonist of IGF1R signaling being administered over the course of about 60 min to about 90 min based on the weight of the individual. In certain embodiments, the about 20 mg/kg of the antagonist of IGF1R signaling is administered to the individual once every 3 weeks for about 7 times.

Also described herein in another aspect is a method of treating an individual afflicted with an insulin-like growth factor 1 receptor (IGF1R) associated disorder wherein the individual is also afflicted with diabetes, the method comprising: (a) administering one or more anti-diabetic drugs to lower blood glucose levels; and (b) administering a dose of the antagonist of IGF1R signaling to the individual. In certain embodiments, the IGF1R associated disorder comprises thyroid eye disease. In certain embodiments, the antagonist of IGF1R signaling comprises an antibody that binds IGF1R. In certain embodiments, the antibody that binds IGF1R comprises ganitumab, figitumumab, MEDI-573, cixutumumab, dalotuzumab, robatumumab, AVE1642, B1113022, xentuzumab, istiratumab, or linsitinib. In certain embodiments, the antibody that binds IGF1R comprises teprotumumab. In certain embodiments, the antagonist of IGF1R signaling comprises a small molecule that inhibits IGF1R signaling. In certain embodiments, the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, or A-928605. In certain embodiments, the antibody that binds IGF1R comprises: (a) a heavy chain comprising: (i) a first heavy chain complementarity determining region (HCDR1) comprising an amino acid sequence according to SEQ ID No.: 5; (ii) a second heavy chain complementarity determining region (HCDR2) comprising an amino acid sequence according to SEQ ID No.: 6; and (iii) a third heavy chain complementarity determining region (HCDR3) comprising an amino acid sequence according to SEQ ID No.: 7; and (b) a light chain comprising: (i) a first light chain complementarity determining region (LCDR1) comprising an amino acid sequence according to SEQ ID No.: 8; (ii) a second light chain complementarity determining region (LCDR2) comprising an amino acid sequence according to SEQ ID No.: 9; and (iii) a third light chain complementarity determining region (LCDR3) comprising an amino acid sequence according to SEQ ID No.: 10. In certain embodiments, the antibody that binds IGF1R comprises: (a) a heavy chain variable domain comprising an amino acid sequence having at least 85%, 90%, or 95% sequence identity to SEQ ID No.: 3; and (b) a light chain variable domain comprising an amino acid sequence having at least 85%, 90%, or 95% sequence identity to SEQ ID No.: 4. In certain embodiments, the antibody that binds IGF1R comprises: (a) a heavy chain variable domain comprising an amino acid sequence according to SEQ ID No.: 3; and (b) a light chain variable domain comprising an amino acid sequence according to SEQ ID No.: 4. In certain embodiments, the antibody that binds IGF1R comprises: (a) a heavy chain comprising an amino acid sequence having at least 85%, 90%, or 95% sequence identity to SEQ ID No.: 1; and (b) a light chain comprising an amino acid sequence having at least 85%, 90%, or 95% sequence identity to SEQ ID No.: 2. V the antibody that binds IGF1R comprises: (a) a heavy chain comprising an amino acid sequence according to SEQ ID No.: 1; and (b) a light chain comprising an amino acid sequence according to SEQ ID No.: 2. In certain embodiments, the one or more anti-diabetic drugs are selected from the list consisting of insulin, metformin, pioglitazone, rosiglitazone, acarbose, miglitol, bromocriptine, alogliptin, linagliptin, saxagliptin, sitagliptin, dulaglutide, exenatide, liraglutide, lixisenatide, semaglutide, tirzepatide, nateglinide, repaglinide, canagliflozin, dapagliflozin, empagliflozin, ertugliflozin, glimepiride, gliclazide, glipizide, and combinations thereof. In certain embodiments, blood glucose levels are established by a measurement of hemoglobin A1c (HbA1c), an oral glucose tolerance test, a measurement of fasting blood glucose, a measurement of non-fasting blood glucose, or any combination thereof. In certain embodiments, the administering comprises intravenous infusion. In certain embodiments, dose comprises about 10 mg/kg of the antagonist of IGF1R signaling being administered over the course of about 60 min to about 90 min based on the weight of the individual. In certain embodiments, the dose comprises about 20 mg/kg of the antagonist of IGF1R signaling being administered over the course of about 60 min to about 90 min based on the weight of the individual. In certain embodiments, the about 20 mg/kg of the antagonist of IGF1R signaling is administered to the individual once every 3 weeks for about 7 times.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example of a method for treatment of an individual afflicted with an IGF1R associated disorder with an antagonist of IGF1R signaling.

### DETAILED DESCRIPTION

The present disclosure employs, unless otherwise indicated, conventional biological and medical techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The term “antibody” herein is used in the broadest sense and includes monoclonal antibodies, and includes intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As described herein a “test for blood glucose control” is any test capable of accurately measuring the blood glucose in an individual. Such tests may utilize a personal glucose monitor such as those compatible with diabetic test strips or insulin pumps; continuous glucose monitors, and laboratory glucose analyzers and autoanalyzer's used by hospitals and laboratory contractors. Such tests for blood glucose control include the measurement of, without limitation fasting blood glucose, non-fasting blood glucose, oral glucose tolerance tests, or the measure of glycated Hemoglobin A1c (HbA1c).

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition. As used herein with respect to thyroid eye disease treating can result in a reduction of Clinical Activity Score (CAS). This reduction could be by 1, 2, 3, 4, 5, 6, or more points.

Normal blood glucose control, when not present, may be achieved through any appropriate means including, but not limited to, diet, exercise, or treatment with biguanide, insulin, DPP4 inhibitors, GLP-1 receptor agonists, meglitinides, sulfonylureas, thiazolidinediones, SGLT2 inhibitors, Alpha-glucosidase inhibitors, or any combination thereof. Normal blood glucose can be established by doctor or patient self-administration of insulin prior to administration of an IGF1R inhibitor.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

### Methods and Composition for Treatment of IGF1R Associated Disorders

The insulin-like growth factor 1 receptor (IGF1R) is expressed on the surface of a number of cell types. The IGF1R belongs to the insulin receptor family and has tyrosine kinase activity. The IGF1R signaling is involved in disease disorders, including thyroid eye disease (TED), also known as Graves' eye disease, Graves' ophthalmopathy, Graves' orbitopathy, thyroid-associated ophthalmopathy (TAO). The thyroid eye disease is an autoimmune disease and may cause inflammation and damage to the muscle and connective tissues of the eyes. Symptoms of the thyroid eye disease may include eye swelling, disfigurement of the eye socket, poor vision, and blindness. An IGF1R associated disorder can be treated with an antagonist of IGF1R signaling.

Thyroid eye disease can be assessed by a clinical activity score (CAS) described by Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mourits et al., Clinical Endocrinology, 1997. 47, no. 1, 9-14). CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present (1 point) or absent (0 points). The score at each efficacy assessment is the sum of all items present; giving a range of 0-7, where 0 or 1 constitutes inactive disease and 7 severe active ophthalmopathy. A CAS score can differentiate between active and inactive TED. Quality of life for individuals can be measured using self-assessment questionnaires such as with the Graves' Ophthalmopathy Quality of Life (GO-QoL) questionnaire. See e.g., Terwee C B, Gerding M N, Dekker F W, Prummel M F, Wiersinga W M. Development of a disease specific quality of life questionnaire for patients with Graves' ophthalmopathy: the GO-QoL. Br J Ophthalmol. 1998 July; 82(7):773-9.

In certain embodiments individuals are also selected for treatment by CAS an individual with a lower CAS may delay treatment. In certain embodiments, an individual with a CAS of 3 or less, 2 or less, 1 or less, or 0. May delay treatment until normal blood glucose is established. An individual with inactive or non-inflammatory TED may delay treatment until normal blood glucose control is established.

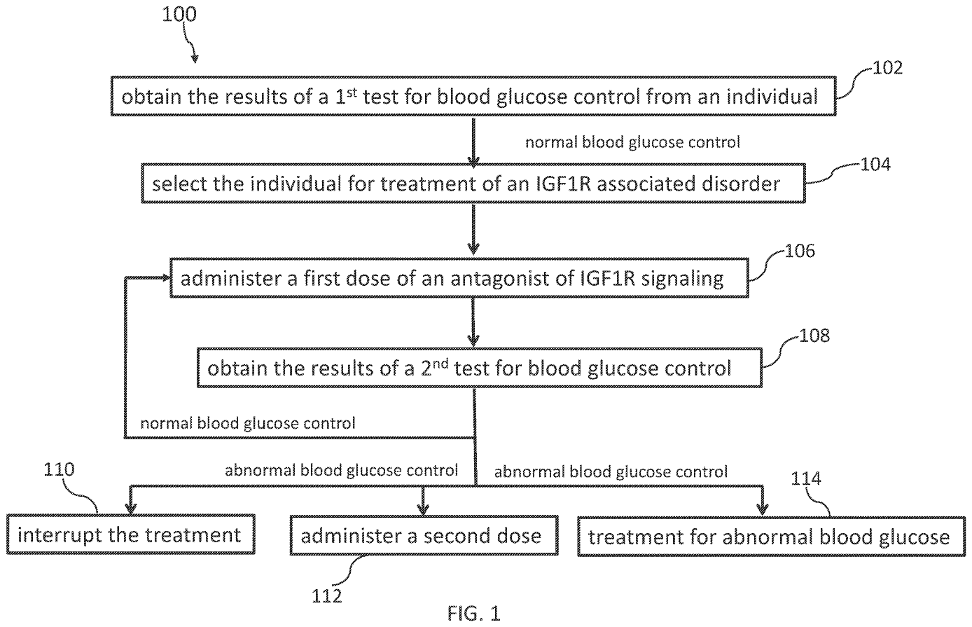

In one aspect, provided herein is a method for diagnosis, treatment, or monitoring of an individual afflicted with an insulin-like growth factor 1 receptor (IGF1R) associated disorder. In some embodiments, the method comprises one or more steps disclosed herein. In some embodiments, the method comprises one or more, but not all, of the steps disclosed herein. FIG. 1 is an illustration of one embodiment of the method 100. The processes, steps, compositions, and components of the method 100 are for illustrative purpose only and should not be construed in any way to limit the scope of the method disclosed herein.

In some embodiments, the method comprises a step 102 of obtaining the results of a first test for blood glucose control from an individual afflicted with an IGF1R associated disorder. In some embodiments, the method comprises a step 104 of selecting the individual afflicted with the IGF1R associated disorder for treatment with an antagonist of IGF1R signaling. In some embodiments, the method comprises selecting the individual afflicted with the IGF1R associated disorder for treatment with the antagonist of IGF1R signaling based on the results of the first test for blood glucose control. In some embodiments, the method comprises selecting the individual afflicted with the IGF1R associated disorder for treatment with the antagonist of IGF1R signaling if the individual exhibits normal blood glucose control by the first test for blood glucose control. In some embodiments, the method comprises selecting the individual afflicted with the IGF1R associated disorder for treatment with the antagonist of IGF1R signaling if the individual exhibits abnormal blood glucose control by the first test for blood glucose control. In some embodiments, the method comprises treating the individual for abnormal blood glucose control before selecting the individual afflicted with the IGF1R associated disorder for treatment with the antagonist of IGF1R signaling. In some embodiments, the treatment for abnormal blood glucose control comprises any suitable treatment, including but not limited to insulin treatment.

In some embodiments, the method comprises a step 106 of administering a first dose of the antagonist of IGF1R signaling to the individual. In some embodiments, the method comprises administering the first dose of the antagonist of IGF1R signaling to the individual based on the results of the first test for blood glucose control. In some embodiments, the method comprises administering the first dose of the antagonist of IGF1R signaling to the individual if the individual exhibits normal blood glucose control by the first test for blood glucose control. In some embodiments, the method comprises administering the first dose of the antagonist of IGF1R signaling to the individual if the individual exhibits abnormal blood glucose control by the first test for blood glucose control. In some embodiments, the method comprises treating the individual for abnormal blood glucose control before administering the first dose of the antagonist of IGF1R signaling to the individual.

In some embodiments, the method comprises a step 108 of obtaining the results of a second test for blood glucose control from the individual being treated with the first dose of the antagonist of IGF1R signaling. In some embodiments, the method comprises a step 110 of interrupting the treatment of the individual with the first dose of the antagonist of IGF1R signaling. In some embodiments, the method comprises interrupting the treatment of the individual with the first dose of the antagonist of IGF1R signaling based on the results of the second test for blood glucose control. In some embodiments, the method comprises interrupting the treatment of the individual with the first dose of the antagonist of IGF1R signaling if the individual exhibits abnormal blood glucose control by the second test for blood glucose control. In some embodiments, the method comprises interrupting the treatment of the individual with the first dose of the antagonist of IGF1R signaling if the individual exhibits normal blood glucose control by the second test for blood glucose control.

In some embodiments, the method comprises a step 112 of administering a second dose of the antagonist of IGF1R signaling to the individual. In some embodiments, the method comprises administering the second dose of the antagonist of IGF1R signaling to the individual based on the results of the second test for blood glucose control. In some embodiments, the method comprises administering the second dose of the antagonist of IGF1R signaling to the individual if the individual exhibits abnormal blood glucose control by the second test for blood glucose control. In some embodiments, the method comprises administering the second dose of the antagonist of IGF1R signaling to the individual if the individual exhibits normal blood glucose control by the second test for blood glucose control. In some embodiments, the second dose of the antagonist of IGF1R signaling is the same as the first dose of the antagonist of IGF1R signaling. In some embodiments, the second dose of the antagonist of IGF1R signaling is different from the first dose of the antagonist of IGF1R signaling. In some embodiments, the second dose of the antagonist of IGF1R signaling is higher than the first dose of the antagonist of IGF1R signaling. In some embodiments, the second dose of the antagonist of IGF1R signaling is lower than the first dose of the antagonist of IGF1R signaling. In some embodiments, the second test for blood glucose control is a different test from the first test for blood glucose control. In some embodiments, the second test for blood glucose control is the same test as the first test for blood glucose control. In some embodiments, the method comprises a step 114 of treating the individual for abnormal blood glucose control if the individual exhibits abnormal blood glucose control by the second test for blood glucose control. In some embodiments, the treatment for abnormal blood glucose control comprises any suitable treatment, including but not limited to insulin treatment.

In some embodiments, the method 100, or one or more steps, or processes of the method 100 disclosed herein may be carried out using a pharmaceutical composition comprising an antagonist of IGF1R signaling, as described below. In some embodiments, the pharmaceutical composition comprises an effective amount of the antagonist of IGF1R signaling. In some embodiments, the method comprises obtaining the results of a first test for blood glucose control from an individual afflicted with an IGF1R associated disorder. In some embodiments, the method comprises selecting the individual afflicted with the IGF1R associated disorder for treatment with a pharmaceutical composition comprising an antagonist of IGF1R signaling. In some embodiments, the method comprises selecting the individual afflicted with the IGF1R associated disorder for treatment with the pharmaceutical composition comprising the antagonist of IGF1R signaling based on the results of the first test for blood glucose control. In some embodiments, the method comprises selecting the individual afflicted with the IGF1R associated disorder for treatment with the pharmaceutical composition comprising the antagonist of IGF1R signaling if the individual exhibits normal blood glucose control by the first test for blood glucose control. In some embodiments, the method comprises selecting the individual afflicted with the IGF1R associated disorder for treatment with the pharmaceutical composition comprising the antagonist of IGF1R signaling if the individual exhibits abnormal blood glucose control by the first test for blood glucose control. In some embodiments, the method comprises treating the individual for abnormal blood glucose control before selecting the individual afflicted with the IGF1R associated disorder for treatment with the pharmaceutical composition comprising the antagonist of IGF1R signaling. In some embodiments, the treatment for abnormal blood glucose control comprises any suitable treatment, including but not limited to insulin treatment.

In some embodiments, the method comprises administering a first dose of the pharmaceutical composition comprising the antagonist of IGF1R signaling to the individual. In some embodiments, the method comprises administering the first dose of the pharmaceutical composition comprising the antagonist of IGF1R signaling to the individual based on the results of the first test for blood glucose control. In some embodiments, the method comprises administering the first dose of the pharmaceutical composition comprising the antagonist of IGF1R signaling to the individual if the individual exhibits normal blood glucose control by the first test for blood glucose control. In some embodiments, the method comprises administering the first dose of the pharmaceutical composition comprising the antagonist of IGF1R signaling to the individual if the individual exhibits abnormal blood glucose control by the first test for blood glucose control. In some embodiments, the method comprises treating the individual for abnormal blood glucose control before administering the first dose of the pharmaceutical composition comprising the antagonist of IGF1R signaling to the individual.

In some embodiments, the method comprises obtaining the results of a second test for blood glucose control from the individual being treated with the first dose of the pharmaceutical composition comprising the antagonist of IGF1R signaling. In some embodiments, the method comprises interrupting the treatment of the individual with the first dose of the pharmaceutical composition comprising the antagonist of IGF1R signaling. In some embodiments, the method comprises interrupting the treatment of the individual with the first dose of the pharmaceutical composition comprising the antagonist of IGF1R signaling based on the results of the second test for blood glucose control. In some embodiments, the method comprises interrupting the treatment of the individual with the first dose of the pharmaceutical composition comprising the antagonist of IGF1R signaling if the individual exhibits abnormal blood glucose control by the second test for blood glucose control. In some embodiments, the method comprises interrupting the treatment of the individual with the first dose of the pharmaceutical composition comprising the antagonist of IGF1R signaling if the individual exhibits normal blood glucose control by the second test for blood glucose control.

In some embodiments, the method comprises administering a second dose of the pharmaceutical composition comprising the antagonist of IGF1R signaling to the individual. In some embodiments, the method comprises administering the second dose of the pharmaceutical composition comprising the antagonist of IGF1R signaling to the individual based on the results of the second test for blood glucose control. In some embodiments, the method comprises administering the second dose of the pharmaceutical composition comprising the antagonist of IGF1R signaling to the individual if the individual exhibits abnormal blood glucose control by the second test for blood glucose control. In some embodiments, the method comprises administering the second dose of the pharmaceutical composition comprising the antagonist of IGF1R signaling to the individual if the individual exhibits normal blood glucose control by the second test for blood glucose control. In some embodiments, the second dose of the pharmaceutical composition comprising the antagonist of IGF1R signaling is the same as the first dose of the pharmaceutical composition comprising the antagonist of IGF1R signaling. In some embodiments, the second dose of the pharmaceutical composition comprising the antagonist of IGF1R signaling is different from the first dose of the pharmaceutical composition comprising the antagonist of IGF1R signaling. In some embodiments, the second dose of the pharmaceutical composition comprising the antagonist of IGF1R signaling is higher than the first dose of the pharmaceutical composition comprising the antagonist of IGF1R signaling. In some embodiments, the second dose of the pharmaceutical composition comprising the antagonist of IGF1R signaling is lower than the first dose of the pharmaceutical composition comprising the antagonist of IGF1R signaling. In some embodiments, the second test for blood glucose control is a different test from the first test for blood glucose control. In some embodiments, the second test for blood glucose control is the same test as the first test for blood glucose control. In some embodiments, the method comprises treating the individual for abnormal blood glucose control if the individual exhibits abnormal blood glucose control by the second test for blood glucose control. In some embodiments, the treatment for abnormal blood glucose control comprises any suitable treatment, including but not limited to insulin treatment.

In some embodiments, the IGF1R associated disorder comprises the thyroid eye disease (TED). In some embodiments, the thyroid eye disease is Graves' eye disease, Graves' ophthalmopathy, Graves' orbitopathy, or thyroid-associated ophthalmopathy (TAO). In some embodiments, the antagonist of IGF1R signaling inhibits, reduces, blocks, or prevents the IGF1R signaling. In some embodiments, the antagonist of IGF1R signaling comprises an antibody that binds IGF1R. In some embodiments, the binding of the antagonist of IGF1R signaling to IGF1R inhibits, reduces, blocks, or prevents the IGF1R signaling. In some embodiments, the antibody that binds IGF1R comprises one or more selected from the group comprising ganitumab, figitumumab, MEDI-573, cixutumumab, dalotuzumab, robatumumab, AVE1642, BIIB022, xentuzumab, istiratumab, linsitinib, and teprotumumab. In some embodiments, the antibody that binds IGF1R comprises two selected from the group comprising ganitumab, figitumumab, MEDI-573, cixutumumab, dalotuzumab, robatumumab, AVE1642, BIIB022, xentuzumab, istiratumab, linsitinib, and teprotumumab. In some embodiments, the antibody that binds IGF1R comprises three selected from the group comprising ganitumab, figitumumab, MEDI-573, cixutumumab, dalotuzumab, robatumumab, AVE1642, BIIB022, xentuzumab, istiratumab, linsitinib, and teprotumumab. In some embodiments, the antibody that binds IGF1R comprises four or more selected from the group comprising ganitumab, figitumumab, MEDI-573, cixutumumab, dalotuzumab, robatumumab, AVE1642, BIIB022, xentuzumab, istiratumab, linsitinib, and teprotumumab. In some embodiments, the antibody that binds IGF1R comprises teprotumumab, also known as TEPEZZA® (teprotumumab-trbw). In some embodiments, the antagonist of IGF1R signaling comprises a small molecule that inhibits reduces, blocks, or prevents the IGF1R signaling. In some embodiments, the small molecule comprises one or more selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605. In some embodiments, the small molecule comprises two selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605. In some embodiments, the small molecule comprises three selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605. In some embodiments, the small molecule comprises four or more selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605.

In some embodiments, the antagonist of IGF1R signaling comprises: (a) an antibody that binds IGF1R; and (b) a small molecule that inhibits reduces, blocks, or prevents the IGF1R signaling. In some embodiments, the antagonist of IGF1R signaling comprises (a) an antibody that binds IGF1R comprising one or more selected from the group comprising ganitumab, figitumumab, MEDI-573, cixutumumab, dalotuzumab, robatumumab, AVE1642, BI113022, xentuzumab, istiratumab, linsitinib, and teprotumumab; and (b) a small molecule that inhibits reduces, blocks, or prevents the IGF1R signaling comprising one or more selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605. In some embodiments, the antagonist of IGF1R signaling comprises (a) an antibody that binds IGF1R comprising teprotumumab; and (b) a small molecule that inhibits reduces, blocks, or prevents the IGF1R signaling comprising one or more selected from the group comprising picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, and A-928605.

In some embodiments, the antibody that binds IGF1R comprises an amino acid sequence according to any one listed in Table 1.

TABLE 1
Sequences of Teprotumumab

| SEQ | | |
|---|---|---|
| ID | | |
| No. | Description | Amino Acid Sequences |
| 1 | Teprotumumab | QVELVESGGGVVQPGRSQRLSCAASGFT |
| | HC¹ | FSSYGMHWVRQAPGKGLEWVAIIWFDGS |
| | | STYYADSVRGRFTISRDNSKNTLYLQMN |
| | | SLRAEDTAVYFCARELGRRYFDLWGRGT |
| | | LVSVSSASTKGPSVFPLAPSSKSTSGGT |
| | | AALGCLVKDYFPEPVTVSWNSGALTSGV |
| | | HTFPAVLQSSGLYSLSSVVTVPSSSLGT |
| | | QTYICNVNHKPSNTKVDKKVEPKSCDKT |
| | | HTCPPCPAPELLGGPSVFLFPPKPKDTL |
| | | MISRTPEVTCVVVDVSHEDPEVKFNWYV |
| | | DGVEVHNAKTKPREEQYNSTYRVVSVLT |
| | | VLHQDWLNGKEYKCKVSNKALPAPIEKT |
| | | ISKAKGQPREPQVYTLPPSRDELTKNQV |
| | | SLTCLVKGFYPSDIAVEWESNGQPENNY |
| | | KTTPPVLDSDGSFFLYSKLTVDKSRWQQ |
| | | GNVFSCSVMHEALHNHYTQKSLSLSPGK |
| 2 | Teprotumumab | EIVLTQSPATLSLSPGERATLSCRASQS |
| | LC² | VSSYLAWYQQKPGQAPRLLIYDASKRAT |
| | | GIPARFSGSGSGTDFTLTISSLEPEDFA |
| | | VYYCQQRSKWPPWTFGQGTKVESKRTVA |
| | | APSVFIFPPSDEQLKSGTASVVCLLNNF |
| | | YPREAKVQWKVDNALQSGNSQESVTEQD |
| | | SKDSTYSLSSTLTLSKADYEKHKVYACE |
| | | VTHQGLSSPVTKSFNRGEC |
| 3 | Teprotumumab | QVELVESGGGVVQPGRSQRLSCAASGFT |
| | VH³ | FSSYGMHWVRQAPGKGLEWVAIIWFDGS |
| | | STYYADSVRGRFTISRDNSKNTLYLQMN |
| | | SLRAEDTAVYFCARELGRRYFDLWGRGT |
| | | LVSVSS |
| 4 | Teprotumumab | EIVLTQSPATLSLSPGERATLSCRASQS |
| | VL⁴ | VSSYLAWYQQKPGQAPRLLIYDASKRAT |
| | | GIPARFSGSGSGTDFTLTISSLEPEDFA |
| | | VYYCQQRSKWPPWTFGQGTKVESK |
| 5 | Teprotumumab | SYGMH |
| | HCDR1⁵ | |
| 6 | Teprotumumab | IIWFDGSSTYYADSVRG |
| | HCDR2⁶ | |
| 7 | Teprotumumab | ELGRRYFDL |
| | HCDR3⁷ | |
| 8 | Teprotumumab | RASQSVSSYLA |
| | LCDR1⁸ | |
| 9 | Teprotumumab | DASKRAT |
| | LCDR2⁹ | |
| 10 | Teprotumumab | QQRSKWPPWTF |
| | LCDR3¹⁰ | |

HC¹: Heavy Chain
LC²: Light Chain
VH³: Heavy Chain Variable Region
VL⁴: Light Chain Variable Region
HCDR1⁵: First Heavy Chain Complementarity Determining Region
HCDR2⁶: Second Heavy Chain Complementarity Determining Region
HCDR3⁷: Third Heavy Chain Complementarity Determining Region
LCDR1⁸: First Light Chain Complementarity Determining Region
LCDR2⁹: Second Light Chain Complementarity Determining Region
LCDR3¹⁰: Third Light Chain Complementarity Determining Region

In some embodiments, the antibody that binds IGF1R comprises a heavy chain comprising a first heavy chain complementarity determining region (HCDR1) comprising an amino acid sequence according to SEQ ID No.: 5. In some embodiments, the antibody that binds IGF1R comprises a heavy chain comprising a second heavy chain complementarity determining region (HCDR2) comprising an amino acid sequence according to SEQ ID No.: 6. In some embodiments, the antibody that binds IGF1R comprises a heavy chain comprising a third heavy chain complementarity determining region (HCDR3) comprising an amino acid sequence according to SEQ ID No.: 7. In some embodiments, the antibody that binds IGF1R comprises a heavy chain comprising: (a) a HCDR1 comprising an amino acid sequence according to SEQ ID No.: 5; (b) a HCDR2 comprising an amino acid sequence according to SEQ ID No.: 6; and (c) a HCDR3 comprising an amino acid sequence according to SEQ ID No.: 7. In some embodiments, the antibody that binds IGF1R comprises a light chain comprising a first light chain complementarity determining region (LCDR1) comprising an amino acid sequence according to SEQ ID No.: 8. In some embodiments, the antibody that binds IGF1R comprises a light chain comprising a second light chain complementarity determining region (LCDR2) comprising an amino acid sequence according to SEQ ID No.: 9. In some embodiments, the antibody that binds IGF1R comprises a light chain comprising a third light chain complementarity determining region (LCDR3) comprising an amino acid sequence according to SEQ ID No.: 10. In some embodiments, the antibody that binds IGF1R comprises a light chain comprising: (a) a LCDR1 comprising an amino acid sequence according to SEQ ID No.: 8; (b) a LCDR2 comprising an amino acid sequence according to SEQ ID No.: 9; and (c) a LCDR3 comprising an amino acid sequence according to SEQ ID No.: 10. In some embodiments, the antibody that binds IGF1R comprises: (a) a heavy chain comprising: (i) a HCDR1 comprising an amino acid sequence according to SEQ ID No.: 5; (ii) a HCDR2 comprising an amino acid sequence according to SEQ ID No.: 6; and (iii) a HCDR3 comprising an amino acid sequence according to SEQ ID No.: 7; and (b) a light chain comprising: (i) a LCDR1 comprising an amino acid sequence according to SEQ ID No.: 8; (ii) a LCDR2 comprising an amino acid sequence according to SEQ ID No.: 9; and (iii) a LCDR3 comprising an amino acid sequence according to SEQ ID No.: 10.

In some embodiments, the antibody that binds IGF1R comprises a heavy chain variable domain (V_{H}) comprising an amino acid sequence having at least 75% sequence identity to SEQ ID No.: 3. In some embodiments, the antibody that binds IGF1R comprises a heavy chain variable domain (V_{H}) comprising an amino acid sequence having at least 76% sequence identity to SEQ ID No.: 3. In some embodiments, the antibody that binds IGF1R comprises a heavy chain variable domain (V_{H}) comprising an amino acid sequence having at least 77% sequence identity to SEQ ID No.: 3. In some embodiments, the antibody that binds IGF1R comprises a heavy chain variable domain (V_{H}) comprising an amino acid sequence having at least 78% sequence identity to SEQ ID No.: 3. In some embodiments, the antibody that binds IGF1R comprises a heavy chain variable domain (V_{H}) comprising an amino acid sequence having at least 79% sequence identity to SEQ ID No.: 3. In some embodiments, the antibody that binds IGF1R comprises a heavy chain variable domain (V_{H}) comprising an amino acid sequence having at least 80% sequence identity to SEQ ID No.: 3. In some embodiments, the antibody that binds IGF1R comprises a heavy chain variable domain (V_{H}) comprising an amino acid sequence having at least 81% sequence identity to SEQ ID No.: 3. In some embodiments, the antibody that binds IGF1R comprises a heavy chain variable domain (V_{H}) comprising an amino acid sequence having at least 82% sequence identity to SEQ ID No.: 3. In some embodiments, the antibody that binds IGF1R comprises a heavy chain variable domain (V_{H}) comprising an amino acid sequence having at least 83% sequence identity to SEQ ID No.: 3. In some embodiments, the antibody that binds IGF1R comprises a heavy chain variable domain (V_{H}) comprising an amino acid sequence having at least 84% sequence identity to SEQ ID No.: 3. In some embodiments, the antibody that binds IGF1R comprises a heavy chain variable domain (V_{H}) comprising an amino acid sequence having at least 85% sequence identity to SEQ ID No.: 3. In some embodiments, the antibody that binds IGF1R comprises a heavy chain variable domain (V_{H}) comprising an amino acid sequence having at least 86% sequence identity to SEQ ID No.: 3. In some embodiments, the antibody that binds IGF1R comprises a heavy chain variable domain (V_{H}) comprising an amino acid sequence having at least 87% sequence identity to SEQ ID No.: 3. In some embodiments, the antibody that binds IGF1R comprises a heavy chain variable domain (V_{H}) comprising an amino acid sequence having at least 88% sequence identity to SEQ ID No.: 3. In some embodiments, the antibody that binds IGF1R comprises a heavy chain variable domain (V_{H}) comprising an amino acid sequence having at least 89% sequence identity to SEQ ID No.: 3. In some embodiments, the antibody that binds IGF1R comprises a heavy chain variable domain (V_{H}) comprising an amino acid sequence having at least 90% sequence identity to SEQ ID No.: 3. In some embodiments, the antibody that binds IGF1R comprises a heavy chain variable domain (V_{H}) comprising an amino acid sequence having at least 91% sequence identity to SEQ ID No.: 3. In some embodiments, the antibody that binds IGF1R comprises a heavy chain variable domain (V_{H}) comprising an amino acid sequence having at least 92% sequence identity to SEQ ID No.: 3. In some embodiments, the antibody that binds IGF1R comprises a heavy chain variable domain (V_{H}) comprising an amino acid sequence having at least 93% sequence identity to SEQ ID No.: 3. In some embodiments, the antibody that binds IGF1R comprises a heavy chain variable domain (V_{H}) comprising an amino acid sequence having at least 94% sequence identity to SEQ ID No.: 3. In some embodiments, the antibody that binds IGF1R comprises a heavy chain variable domain (V_{H}) comprising an amino acid sequence having at least 95% sequence identity to SEQ ID No.: 3. In some embodiments, the antibody that binds IGF1R comprises a heavy chain variable domain (V_{H}) comprising an amino acid sequence having at least 96% sequence identity to SEQ ID No.: 3. In some embodiments, the antibody that binds IGF1R comprises a heavy chain variable domain (V_{H}) comprising an amino acid sequence having at least 97% sequence identity to SEQ ID No.: 3. In some embodiments, the antibody that binds IGF1R comprises a heavy chain variable domain (V_{H}) comprising an amino acid sequence having at least 98% sequence identity to SEQ ID No.: 3. In some embodiments, the antibody that binds IGF1R comprises a heavy chain variable domain (V_{H}) comprising an amino acid sequence having at least 99% sequence identity to SEQ ID No.: 3. In some embodiments, the antibody that binds IGF1R comprises a heavy chain variable domain (V_{H}) comprising an amino acid sequence according to SEQ ID No.: 3.

In some embodiments, the antibody that binds IGF1R comprises a light chain variable domain (V_{L}) comprising an amino acid sequence having at least 75% sequence identity to SEQ ID No.: 4. In some embodiments, the antibody that binds IGF1R comprises a light chain variable domain (V_{L}) comprising an amino acid sequence having at least 76% sequence identity to SEQ ID No.: 4. In some embodiments, the antibody that binds IGF1R comprises a light chain variable domain (V_{L}) comprising an amino acid sequence having at least 77% sequence identity to SEQ ID No.: 4. In some embodiments, the antibody that binds IGF1R comprises a light chain variable domain (V_{L}) comprising an amino acid sequence having at least 78% sequence identity to SEQ ID No.: 4. In some embodiments, the antibody that binds IGF1R comprises a light chain variable domain (V_{L}) comprising an amino acid sequence having at least 79% sequence identity to SEQ ID No.: 4. In some embodiments, the antibody that binds IGF1R comprises a light chain variable domain (V_{L}) comprising an amino acid sequence having at least 80% sequence identity to SEQ ID No.: 4. In some embodiments, the antibody that binds IGF1R comprises a heavy chain variable domain (V_{H}) comprising an amino acid sequence having at least 81% sequence identity to SEQ ID No.: 3. In some embodiments, the antibody that binds IGF1R comprises a heavy chain variable domain (V_{H}) comprising an amino acid sequence having at least 82% sequence identity to SEQ ID No.: 3. In some embodiments, the antibody that binds IGF1R comprises a heavy chain variable domain (V_{H}) comprising an amino acid sequence having at least 83% sequence identity to SEQ ID No.: 3. In some embodiments, the antibody that binds IGF1R comprises a heavy chain variable domain (V_{H}) comprising an amino acid sequence having at least 84% sequence identity to SEQ ID No.: 3. In some embodiments, the antibody that binds IGF1R comprises a heavy chain variable domain (V_{H}) comprising an amino acid sequence having at least 85% sequence identity to SEQ ID No.: 3. In some embodiments, the antibody that binds IGF1R comprises a heavy chain variable domain (V_{H}) comprising an amino acid sequence having at least 86% sequence identity to SEQ ID No.: 3. In some embodiments, the antibody that binds IGF1R comprises a heavy chain variable domain (V_{H}) comprising an amino acid sequence having at least 87% sequence identity to SEQ ID No.: 3. In some embodiments, the antibody that binds IGF1R comprises a heavy chain variable domain (V_{H}) comprising an amino acid sequence having at least 88% sequence identity to SEQ ID No.: 3. In some embodiments, the antibody that binds IGF1R comprises a heavy chain variable domain (V_{H}) comprising an amino acid sequence having at least 89% sequence identity to SEQ ID No.: 3. In some embodiments, the antibody that binds IGF1R comprises a heavy chain variable domain (V_{H}) comprising an amino acid sequence having at least 90% sequence identity to SEQ ID No.: 3. In some embodiments, the antibody that binds IGF1R comprises a heavy chain variable domain (V_{H}) comprising an amino acid sequence having at least 91% sequence identity to SEQ ID No.: 3. In some embodiments, the antibody that binds IGF1R comprises a heavy chain variable domain (V_{H}) comprising an amino acid sequence having at least 92% sequence identity to SEQ ID No.: 3. In some embodiments, the antibody that binds IGF1R comprises a heavy chain variable domain (V_{H}) comprising an amino acid sequence having at least 93% sequence identity to SEQ ID No.: 3. In some embodiments, the antibody that binds IGF1R comprises a heavy chain variable domain (V_{H}) comprising an amino acid sequence having at least 94% sequence identity to SEQ ID No.: 3. In some embodiments, the antibody that binds IGF1R comprises a heavy chain variable domain (V_{H}) comprising an amino acid sequence having at least 95% sequence identity to SEQ ID No.: 3. In some embodiments, the antibody that binds IGF1R comprises a heavy chain variable domain (V_{H}) comprising an amino acid sequence having at least 96% sequence identity to SEQ ID No.: 3. In some embodiments, the antibody that binds IGF1R comprises a heavy chain variable domain (V_{H}) comprising an amino acid sequence having at least 97% sequence identity to SEQ ID No.: 3. In some embodiments, the antibody that binds IGF1R comprises a heavy chain variable domain (V_{H}) comprising an amino acid sequence having at least 98% sequence identity to SEQ ID No.: 3. In some embodiments, the antibody that binds IGF1R comprises a heavy chain variable domain (V_{H}) comprising an amino acid sequence having at least 99% sequence identity to SEQ ID No.: 3. In some embodiments, the antibody that binds IGF1R comprises a light chain variable domain (V_{L}) comprising an amino acid sequence according to SEQ ID No.: 4.

In some embodiments, the antibody that binds IGF1R comprises a V_{H} comprising an amino acid sequence having at least 75% sequence identity to SEQ ID No.: 3 and a V_{L} comprising an amino acid sequence having at least 75% sequence identity to SEQ ID No.: 4. In some embodiments, the antibody that binds IGF1R comprises a V_{H} comprising an amino acid sequence having at least 76% sequence identity to SEQ ID No.: 3 and a V_{L} comprising an amino acid sequence having at least 76% sequence identity to SEQ ID No.: 4. In some embodiments, the antibody that binds IGF1R comprises a V_{H} comprising an amino acid sequence having at least 77% sequence identity to SEQ ID No.: 3 and a V_{L} comprising an amino acid sequence having at least 77% sequence identity to SEQ ID No.: 4. In some embodiments, the antibody that binds IGF1R comprises a V_{H} comprising an amino acid sequence having at least 78% sequence identity to SEQ ID No.: 3 and a V_{L} comprising an amino acid sequence having at least 78% sequence identity to SEQ ID No.: 4. In some embodiments, the antibody that binds IGF1R comprises a V_{H} comprising an amino acid sequence having at least 79% sequence identity to SEQ ID No.: 3 and a V_{L} comprising an amino acid sequence having at least 79% sequence identity to SEQ ID No.: 4. In some embodiments, the antibody that binds IGF1R comprises a V_{H} comprising an amino acid sequence having at least 80% sequence identity to SEQ ID No.: 3 and a V_{L} comprising an amino acid sequence having at least 80% sequence identity to SEQ ID No.: 4. In some embodiments, the antibody that binds IGF1R comprises a V_{H} comprising an amino acid sequence having at least 81% sequence identity to SEQ ID No.: 3 and a V_{L} comprising an amino acid sequence having at least 81% sequence identity to SEQ ID No.: 4. In some embodiments, the antibody that binds IGF1R comprises a V_{H} comprising an amino acid sequence having at least 82% sequence identity to SEQ ID No.: 3 and a V_{L} comprising an amino acid sequence having at least 82% sequence identity to SEQ ID No.: 4. In some embodiments, the antibody that binds IGF1R comprises a V_{H} comprising an amino acid sequence having at least 83% sequence identity to SEQ ID No.: 3 and a V_{L} comprising an amino acid sequence having at least 83% sequence identity to SEQ ID No.: 4. In some embodiments, the antibody that binds IGF1R comprises a V_{H} comprising an amino acid sequence having at least 84% sequence identity to SEQ ID No.: 3 and a V_{L} comprising an amino acid sequence having at least 84% sequence identity to SEQ ID No.: 4. In some embodiments, the antibody that binds IGF1R comprises a V_{H} comprising an amino acid sequence having at least 85% sequence identity to SEQ ID No.: 3 and a V_{L} comprising an amino acid sequence having at least 85% sequence identity to SEQ ID No.: 4. In some embodiments, the antibody that binds IGF1R comprises a V_{H} comprising an amino acid sequence having at least 86% sequence identity to SEQ ID No.: 3 and a V_{L} comprising an amino acid sequence having at least 86% sequence identity to SEQ ID No.: 4. In some embodiments, the antibody that binds IGF1R comprises a V_{H} comprising an amino acid sequence having at least 87% sequence identity to SEQ ID No.: 3 and a V_{L} comprising an amino acid sequence having at least 87% sequence identity to SEQ ID No.: 4. In some embodiments, the antibody that binds IGF1R comprises a V_{H} comprising an amino acid sequence having at least 88% sequence identity to SEQ ID No.: 3 and a V_{L} comprising an amino acid sequence having at least 88% sequence identity to SEQ ID No.: 4. In some embodiments, the antibody that binds IGF1R comprises a V_{H} comprising an amino acid sequence having at least 89% sequence identity to SEQ ID No.: 3 and a V_{L} comprising an amino acid sequence having at least 89% sequence identity to SEQ ID No.: 4. In some embodiments, the antibody that binds IGF1R comprises a V_{H} comprising an amino acid sequence having at least 90% sequence identity to SEQ ID No.: 3 and a V_{L} comprising an amino acid sequence having at least 90% sequence identity to SEQ ID No.: 4. In some embodiments, the antibody that binds IGF1R comprises a V_{H} comprising an amino acid sequence having at least 91% sequence identity to SEQ ID No.: 3 and a V_{L} comprising an amino acid sequence having at least 91% sequence identity to SEQ ID No.: 4. In some embodiments, the antibody that binds IGF1R comprises a V_{H} comprising an amino acid sequence having at least 92% sequence identity to SEQ ID No.: 3 and a V_{L} comprising an amino acid sequence having at least 92% sequence identity to SEQ ID No.: 4. In some embodiments, the antibody that binds IGF1R comprises a V_{H} comprising an amino acid sequence having at least 93% sequence identity to SEQ ID No.: 3 and a V_{L} comprising an amino acid sequence having at least 93% sequence identity to SEQ ID No.: 4. In some embodiments, the antibody that binds IGF1R comprises a V_{H} comprising an amino acid sequence having at least 94% sequence identity to SEQ ID No.: 3 and a V_{L} comprising an amino acid sequence having at least 94% sequence identity to SEQ ID No.: 4. In some embodiments, the antibody that binds IGF1R comprises a V_{H} comprising an amino acid sequence having at least 95% sequence identity to SEQ ID No.: 3 and a V_{L} comprising an amino acid sequence having at least 95% sequence identity to SEQ ID No.: 4. In some embodiments, the antibody that binds IGF1R comprises a V_{H} comprising an amino acid sequence having at least 96% sequence identity to SEQ ID No.: 3 and a V_{L} comprising an amino acid sequence having at least 96% sequence identity to SEQ ID No.: 4. In some embodiments, the antibody that binds IGF1R comprises a V_{H} comprising an amino acid sequence having at least 97% sequence identity to SEQ ID No.: 3 and a V_{L} comprising an amino acid sequence having at least 97% sequence identity to SEQ ID No.: 4. In some embodiments, the antibody that binds IGF1R comprises a V_{H} comprising an amino acid sequence having at least 98% sequence identity to SEQ ID No.: 3 and a V_{L} comprising an amino acid sequence having at least 98% sequence identity to SEQ ID No.: 4. In some embodiments, the antibody that binds IGF1R comprises a V_{H} comprising an amino acid sequence having at least 99% sequence identity to SEQ ID No.: 3 and a V_{L} comprising an amino acid sequence having at least 99% sequence identity to SEQ ID No.: 4. In some embodiments, the antibody that binds IGF1R comprises a V_{H} comprising an amino acid sequence according to SEQ ID No.: 3 and a V_{L} comprising an amino acid sequence according to SEQ ID No.: 4.

In some embodiments, the antibody that binds IGF1R comprises a heavy chain comprising an amino acid sequence having at least 75% sequence identity to SEQ ID No.: 1. In some embodiments, the antibody that binds IGF1R comprises a heavy chain comprising an amino acid sequence having at least 76% sequence identity to SEQ ID No.: 1. In some embodiments, the antibody that binds IGF1R comprises a heavy chain comprising an amino acid sequence having at least 77% sequence identity to SEQ ID No.: 1. In some embodiments, the antibody that binds IGF1R comprises a heavy chain comprising an amino acid sequence having at least 78% sequence identity to SEQ ID No.: 1. In some embodiments, the antibody that binds IGF1R comprises a heavy chain comprising an amino acid sequence having at least 79% sequence identity to SEQ ID No.: 1. In some embodiments, the antibody that binds IGF1R comprises a heavy chain comprising an amino acid sequence having at least 80% sequence identity to SEQ ID No.: 1. In some embodiments, the antibody that binds IGF1R comprises a heavy chain comprising an amino acid sequence having at least 81% sequence identity to SEQ ID No.: 1. In some embodiments, the antibody that binds IGF1R comprises a heavy chain comprising an amino acid sequence having at least 82% sequence identity to SEQ ID No.: 1. In some embodiments, the antibody that binds IGF1R comprises a heavy chain comprising an amino acid sequence having at least 83% sequence identity to SEQ ID No.: 1. In some embodiments, the antibody that binds IGF1R comprises a heavy chain comprising an amino acid sequence having at least 84% sequence identity to SEQ ID No.: 1. In some embodiments, the antibody that binds IGF1R comprises a heavy chain comprising an amino acid sequence having at least 85% sequence identity to SEQ ID No.: 1. In some embodiments, the antibody that binds IGF1R comprises a heavy chain comprising an amino acid sequence having at least 86% sequence identity to SEQ ID No.: 1. In some embodiments, the antibody that binds IGF1R comprises a heavy chain comprising an amino acid sequence having at least 87% sequence identity to SEQ ID No.: 1. In some embodiments, the antibody that binds IGF1R comprises a heavy chain comprising an amino acid sequence having at least 88% sequence identity to SEQ ID No.: 1. In some embodiments, the antibody that binds IGF1R comprises a heavy chain comprising an amino acid sequence having at least 89% sequence identity to SEQ ID No.: 1. In some embodiments, the antibody that binds IGF1R comprises a heavy chain comprising an amino acid sequence having at least 90% sequence identity to SEQ ID No.: 1. In some embodiments, the antibody that binds IGF1R comprises a heavy chain comprising an amino acid sequence having at least 91% sequence identity to SEQ ID No.: 1. In some embodiments, the antibody that binds IGF1R comprises a heavy chain comprising an amino acid sequence having at least 92% sequence identity to SEQ ID No.: 1. In some embodiments, the antibody that binds IGF1R comprises a heavy chain comprising an amino acid sequence having at least 93% sequence identity to SEQ ID No.: 1. In some embodiments, the antibody that binds IGF1R comprises a heavy chain comprising an amino acid sequence having at least 94% sequence identity to SEQ ID No.: 1. In some embodiments, the antibody that binds IGF1R comprises a heavy chain comprising an amino acid sequence having at least 95% sequence identity to SEQ ID No.: 1. In some embodiments, the antibody that binds IGF1R comprises a heavy chain comprising an amino acid sequence having at least 96% sequence identity to SEQ ID No.: 1. In some embodiments, the antibody that binds IGF1R comprises a heavy chain comprising an amino acid sequence having at least 97% sequence identity to SEQ ID No.: 1. In some embodiments, the antibody that binds IGF1R comprises a heavy chain comprising an amino acid sequence having at least 98% sequence identity to SEQ ID No.: 1. In some embodiments, the antibody that binds IGF1R comprises a heavy chain comprising an amino acid sequence having at least 99% sequence identity to SEQ ID No.: 1. In some embodiments, the antibody that binds IGF1R comprises a heavy chain comprising an amino acid sequence according to SEQ ID No.: 1.

In some embodiments, the antibody that binds IGF1R comprises a light chain comprising an amino acid sequence having at least 75% sequence identity to SEQ ID No.: 2. In some embodiments, the antibody that binds IGF1R comprises a light chain comprising an amino acid sequence having at least 76% sequence identity to SEQ ID No.: 2. In some embodiments, the antibody that binds IGF1R comprises a light chain comprising an amino acid sequence having at least 77% sequence identity to SEQ ID No.: 2. In some embodiments, the antibody that binds IGF1R comprises a light chain comprising an amino acid sequence having at least 78% sequence identity to SEQ ID No.: 2. In some embodiments, the antibody that binds IGF1R comprises a light chain comprising an amino acid sequence having at least 79% sequence identity to SEQ ID No.: 2. In some embodiments, the antibody that binds IGF1R comprises a light chain comprising an amino acid sequence having at least 80% sequence identity to SEQ ID No.: 2. In some embodiments, the antibody that binds IGF1R comprises a light chain comprising an amino acid sequence having at least 81% sequence identity to SEQ ID No.: 2. In some embodiments, the antibody that binds IGF1R comprises a light chain comprising an amino acid sequence having at least 82% sequence identity to SEQ ID No.: 2. In some embodiments, the antibody that binds IGF1R comprises a light chain comprising an amino acid sequence having at least 83% sequence identity to SEQ ID No.: 2. In some embodiments, the antibody that binds IGF1R comprises a light chain comprising an amino acid sequence having at least 84% sequence identity to SEQ ID No.: 2. In some embodiments, the antibody that binds IGF1R comprises a light chain comprising an amino acid sequence having at least 85% sequence identity to SEQ ID No.: 2. In some embodiments, the antibody that binds IGF1R comprises a light chain comprising an amino acid sequence having at least 86% sequence identity to SEQ ID No.: 2. In some embodiments, the antibody that binds IGF1R comprises a light chain comprising an amino acid sequence having at least 87% sequence identity to SEQ ID No.: 2. In some embodiments, the antibody that binds IGF1R comprises a light chain comprising an amino acid sequence having at least 88% sequence identity to SEQ ID No.: 2. In some embodiments, the antibody that binds IGF1R comprises a light chain comprising an amino acid sequence having at least 89% sequence identity to SEQ ID No.: 2. In some embodiments, the antibody that binds IGF1R comprises a light chain comprising an amino acid sequence having at least 90% sequence identity to SEQ ID No.: 2. In some embodiments, the antibody that binds IGF1R comprises a light chain comprising an amino acid sequence having at least 91% sequence identity to SEQ ID No.: 2. In some embodiments, the antibody that binds IGF1R comprises a light chain comprising an amino acid sequence having at least 92% sequence identity to SEQ ID No.: 2. In some embodiments, the antibody that binds IGF1R comprises a light chain comprising an amino acid sequence having at least 93% sequence identity to SEQ ID No.: 2. In some embodiments, the antibody that binds IGF1R comprises a light chain comprising an amino acid sequence having at least 94% sequence identity to SEQ ID No.: 2. In some embodiments, the antibody that binds IGF1R comprises a light chain comprising an amino acid sequence having at least 95% sequence identity to SEQ ID No.: 2. In some embodiments, the antibody that binds IGF1R comprises a light chain comprising an amino acid sequence having at least 96% sequence identity to SEQ ID No.: 2. In some embodiments, the antibody that binds IGF1R comprises a light chain comprising an amino acid sequence having at least 97% sequence identity to SEQ ID No.: 2. In some embodiments, the antibody that binds IGF1R comprises a light chain comprising an amino acid sequence having at least 98% sequence identity to SEQ ID No.: 2. In some embodiments, the antibody that binds IGF1R comprises a light chain comprising an amino acid sequence having at least 99% sequence identity to SEQ ID No.: 2. In some embodiments, the antibody that binds IGF1R comprises a light chain comprising an amino acid sequence according to SEQ ID No.: 2.

In some embodiments, the antibody that binds IGF1R comprises a heavy chain comprising an amino acid sequence having at least 75% sequence identity to SEQ ID No.: 1 and a light chain comprising an amino acid sequence having at least 75% sequence identity to SEQ ID No.: 2. In some embodiments, the antibody that binds IGF1R comprises a heavy chain comprising an amino acid sequence having at least 76% sequence identity to SEQ ID No.: 1 and a light chain comprising an amino acid sequence having at least 76% sequence identity to SEQ ID No.: 2. In some embodiments, the antibody that binds IGF1R comprises a heavy chain comprising an amino acid sequence having at least 77% sequence identity to SEQ ID No.: 1 and a light chain comprising an amino acid sequence having at least 77% sequence identity to SEQ ID No.: 2. In some embodiments, the antibody that binds IGF1R comprises a heavy chain comprising an amino acid sequence having at least 78% sequence identity to SEQ ID No.: 1 and a light chain comprising an amino acid sequence having at least 78% sequence identity to SEQ ID No.: 2. In some embodiments, the antibody that binds IGF1R comprises a heavy chain comprising an amino acid sequence having at least 79% sequence identity to SEQ ID No.: 1 and a light chain comprising an amino acid sequence having at least 79% sequence identity to SEQ ID No.: 2. In some embodiments, the antibody that binds IGF1R comprises a heavy chain comprising an amino acid sequence having at least 80% sequence identity to SEQ ID No.: 1 and a light chain comprising an amino acid sequence having at least 80% sequence identity to SEQ ID No.: 2. In some embodiments, the antibody that binds IGF1R comprises a heavy chain comprising an amino acid sequence having at least 81% sequence identity to SEQ ID No.: 1 and a light chain comprising an amino acid sequence having at least 81% sequence identity to SEQ ID No.: 2. In some embodiments, the antibody that binds IGF1R comprises a heavy chain comprising an amino acid sequence having at least 82% sequence identity to SEQ ID No.: 1 and a light chain comprising an amino acid sequence having at least 82% sequence identity to SEQ ID No.: 2. In some embodiments, the antibody that binds IGF1R comprises a heavy chain comprising an amino acid sequence having at least 83% sequence identity to SEQ ID No.: 1 and a light chain comprising an amino acid sequence having at least 83% sequence identity to SEQ ID No.: 2. In some embodiments, the antibody that binds IGF1R comprises a heavy chain comprising an amino acid sequence having at least 84% sequence identity to SEQ ID No.: 1 and a light chain comprising an amino acid sequence having at least 84% sequence identity to SEQ ID No.: 2. In some embodiments, the antibody that binds IGF1R comprises a heavy chain comprising an amino acid sequence having at least 85% sequence identity to SEQ ID No.: 1 and a light chain comprising an amino acid sequence having at least 85% sequence identity to SEQ ID No.: 2. In some embodiments, the antibody that binds IGF1R comprises a heavy chain comprising an amino acid sequence having at least 86% sequence identity to SEQ ID No.: 1 and a light chain comprising an amino acid sequence having at least 86% sequence identity to SEQ ID No.: 2. In some embodiments, the antibody that binds IGF1R comprises a heavy chain comprising an amino acid sequence having at least 87% sequence identity to SEQ ID No.: 1 and a light chain comprising an amino acid sequence having at least 87% sequence identity to SEQ ID No.: 2. In some embodiments, the antibody that binds IGF1R comprises a heavy chain comprising an amino acid sequence having at least 88% sequence identity to SEQ ID No.: 1 and a light chain comprising an amino acid sequence having at least 88% sequence identity to SEQ ID No.: 2. In some embodiments, the antibody that binds IGF1R comprises a heavy chain comprising an amino acid sequence having at least 89% sequence identity to SEQ ID No.: 1 and a light chain comprising an amino acid sequence having at least 89% sequence identity to SEQ ID No.: 2. In some embodiments, the antibody that binds IGF1R comprises a heavy chain comprising an amino acid sequence having at least 90% sequence identity to SEQ ID No.: 1 and a light chain comprising an amino acid sequence having at least 90% sequence identity to SEQ ID No.: 2. In some embodiments, the antibody that binds IGF1R comprises a heavy chain comprising an amino acid sequence having at least 91% sequence identity to SEQ ID No.: 1 and a light chain comprising an amino acid sequence having at least 91% sequence identity to SEQ ID No.: 2. In some embodiments, the antibody that binds IGF1R comprises a heavy chain comprising an amino acid sequence having at least 92% sequence identity to SEQ ID No.: 1 and a light chain comprising an amino acid sequence having at least 92% sequence identity to SEQ ID No.: 2. In some embodiments, the antibody that binds IGF1R comprises a heavy chain comprising an amino acid sequence having at least 93% sequence identity to SEQ ID No.: 1 and a light chain comprising an amino acid sequence having at least 93% sequence identity to SEQ ID No.: 2. In some embodiments, the antibody that binds IGF1R comprises a heavy chain comprising an amino acid sequence having at least 94% sequence identity to SEQ ID No.: 1 and a light chain comprising an amino acid sequence having at least 94% sequence identity to SEQ ID No.: 2. In some embodiments, the antibody that binds IGF1R comprises a heavy chain comprising an amino acid sequence having at least 95% sequence identity to SEQ ID No.: 1 and a light chain comprising an amino acid sequence having at least 95% sequence identity to SEQ ID No.: 2. In some embodiments, the antibody that binds IGF1R comprises a heavy chain comprising an amino acid sequence having at least 96% sequence identity to SEQ ID No.: 1 and a light chain comprising an amino acid sequence having at least 96% sequence identity to SEQ ID No.: 2. In some embodiments, the antibody that binds IGF1R comprises a heavy chain comprising an amino acid sequence having at least 97% sequence identity to SEQ ID No.: 1 and a light chain comprising an amino acid sequence having at least 97% sequence identity to SEQ ID No.: 2. In some embodiments, the antibody that binds IGF1R comprises a heavy chain comprising an amino acid sequence having at least 98% sequence identity to SEQ ID No.: 1 and a light chain comprising an amino acid sequence having at least 98% sequence identity to SEQ ID No.: 2. In some embodiments, the antibody that binds IGF1R comprises a heavy chain comprising an amino acid sequence having at least 99% sequence identity to SEQ ID No.: 1 and a light chain comprising an amino acid sequence having at least 99% sequence identity to SEQ ID No.: 2. In some embodiments, the antibody that binds IGF1R comprises a heavy chain comprising an amino acid sequence according to SEQ ID No.: 1 and a light chain comprising an amino acid sequence according to SEQ ID No.: 2.

In some embodiments, a test for blood glucose control comprises a test that measures the level of glucose in a blood sample. In some embodiments, a test for blood glucose control comprises a test that measures the level of sugar in a blood sample. In some embodiments, when the blood glucose level is within a first range, an individual may be classified as being healthy. In some embodiments, when the blood glucose level is within a second range and each value of the second range is higher than each value within the first range, an individual may be classified as having pre-diabetes. In some embodiments, when the blood glucose level is within a third range and each value of the third range is higher than each value within the second range, an individual may be classified as having diabetes. In some embodiments, the test for blood glucose control comprises any type of test that is has been used for, is being used for, or will be used for measurement of blood glucose level. In some embodiments, the test for blood glucose control comprises any type of test that has been used for, is being used for, or will be used for diagnosis of disorder related to blood glucose, including but not limited to high blood glucose level, hyperglycemia, prediabetes, diabetes, type I diabetes, type II diabetes, gestational diabetes, impaired glucose tolerance, or glucose intolerance. In some embodiments, the test for blood glucose control may be carried out using a blood glucose test kit, a blood glucose testing device, or both. In some embodiments, the test for blood glucose control comprises a measurement of hemoglobin A1c (HbA1c), an oral glucose tolerance test, a measurement of fasting blood glucose, a measurement of non-fasting blood glucose, or any combination thereof. In some embodiments, a test for blood glucose control disclosed herein is applicable to the first test for blood glucose control of the method disclosed herein, the second test for blood glucose control of the method disclosed herein, or both.

In some embodiments, the test for blood glucose control comprises a measurement of hemoglobin A1c (HbA1c) in a blood sample. In some embodiments, the measurement of hemoglobin A1c (HbA1c) in a blood sample is also known as a glycated hemoglobin test, a glycosylated hemoglobin test, a hemoglobin A1C test, or a HbA1c test. In some embodiments, the HbA1c test comprises a measurement of the glycated hemoglobin protein in a blood sample. In some embodiments, the HbA1c test comprises a measurement of an average level of the glycated hemoglobin protein in a blood sample over a period of time. In some embodiments, the HbA1c test comprises a measurement of an average level of the percentage of glycated hemoglobin protein among the total amount of hemoglobin protein in a blood sample over a period of time. In some embodiments, said period of time is about 90 days. In some embodiments, the period of time for the HbA1c test is between about 0 and about 24 months, e.g., about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, or about 24.0 months, or any period of time therebetween. In some embodiments, the period of time for the HbA1c test is between about 0 and about 24 months, between about 0 and about 12 months, between about 0 and 6 months, between about 0 and 5 months, between about 0 and 4 months, between about 0 and 3 months, between about 0 and 2 months, between about 0 and 1 month, between about 1 and 24 months, between about 1 and 12 months, between about 1 and 6 months, between about 1 and 5 months, between about 1 and 4 months, between about 1 and 3 months, between about 1 and 2 months, between about 2 and 24 months, between about 2 and 12 months, between about 2 and 6 months, between about 2 and 5 months, between about 2 and 4 months, between about 2 and 3 months, between about 3 and 24 months, between about 3 and 12 months, between about 3 and 6 months, between about 3 and 5 months, between about 3 and 4 months, between about 4 and 24 months, between about 4 and 12 months, between about 4 and 6 months, between about 4 and 5 months, between about 5 and 24 months, between about 5 and 12 months, or between about 5 and 6 months. In some embodiments, the period of time for the HbA1c test is about 1 month. In some embodiments, the period of time for the HbA1c test is about 2 months. In some embodiments, the period of time for the HbA1c test is about 3 months.

In some embodiments, the individual exhibits normal blood glucose control if the measurement of HbA1c is below about 4.5%. In some embodiments, the individual exhibits normal blood glucose control if the measurement of HbA1c is below about 4.6%. In some embodiments, the individual exhibits normal blood glucose control if the measurement of HbA1c is below about 4.7%. In some embodiments, the individual exhibits normal blood glucose control if the measurement of HbA1c is below about 4.8%. In some embodiments, the individual exhibits normal blood glucose control if the measurement of HbA1c is below about 4.9%. In some embodiments, the individual exhibits normal blood glucose control if the measurement of HbA1c is below about 5.0%. In some embodiments, the individual exhibits normal blood glucose control if the measurement of HbA1c is below about 5.1%. In some embodiments, the individual exhibits normal blood glucose control if the measurement of HbA1c is below about 5.2%. In some embodiments, the individual exhibits normal blood glucose control if the measurement of HbA1c is below about 5.3%. In some embodiments, the individual exhibits normal blood glucose control if the measurement of HbA1c is below about 5.4%. In some embodiments, the individual exhibits normal blood glucose control if the measurement of HbA1c is below about 5.5%. In some embodiments, the individual exhibits normal blood glucose control if the measurement of HbA1c is below about 5.6%. In some embodiments, the individual exhibits normal blood glucose control if the measurement of HbA1c is below about 5.7%. In some embodiments, the individual exhibits normal blood glucose control if the measurement of HbA1c is below about 5.8%. In some embodiments, the individual exhibits normal blood glucose control if the measurement of HbA1c is below about 5.9%. In some embodiments, the individual exhibits normal blood glucose control if the measurement of HbA1c is below about 6.0%. In some embodiments, the individual exhibits normal blood glucose control if the measurement of HbA1c is below about 6.1%. In some embodiments, the individual exhibits normal blood glucose control if the measurement of HbA1c is below about 6.2%. In some embodiments, the individual exhibits normal blood glucose control if the measurement of HbA1c is below about 6.3%. In some embodiments, the individual exhibits normal blood glucose control if the measurement of HbA1c is below about 6.4%. In some embodiments, the individual exhibits normal blood glucose control if the measurement of HbA1c is below about 6.5%. In some embodiments, the individual exhibits normal blood glucose control if the measurement of HbA1c is below about 6.6%. In some embodiments, the individual exhibits normal blood glucose control if the measurement of HbA1c is below about 6.7%. In some embodiments, the individual exhibits normal blood glucose control if the measurement of HbA1c is below about 6.8%. In some embodiments, the individual exhibits normal blood glucose control if the measurement of HbA1c is below about 6.9%. In some embodiments, the individual exhibits normal blood glucose control if the measurement of HbA1c is below about 7.0%. In some embodiments, the individual exhibits normal blood glucose control if the measurement of HbA1c is below about 7.1%. In some embodiments, the individual exhibits normal blood glucose control if the measurement of HbA1c is below about 7.2%. In some embodiments, the individual exhibits normal blood glucose control if the measurement of HbA1c is below about 7.3%. %. In some embodiments, the individual exhibits normal blood glucose control if the measurement of HbA1c is below about 7.4%. In some embodiments, the individual exhibits normal blood glucose control if the measurement of HbA1c is below about 7.5%. In some embodiments, the individual exhibits normal blood glucose control if the measurement of HbA1c is below about 7.6%. In some embodiments, the individual exhibits normal blood glucose control if the measurement of HbA1c is below about 7.7%. In some embodiments, the individual exhibits normal blood glucose control if the measurement of HbA1c is below about 7.8%. In some embodiments, the individual exhibits normal blood glucose control if the measurement of HbA1c is below about 7.9%. In some embodiments, the individual exhibits normal blood glucose control if the measurement of HbA1c is below about 8.0%. In some embodiments, the individual exhibits normal blood glucose control if the measurement of HbA1c is below about 8.5%. In some embodiments, the individual exhibits normal blood glucose control if the measurement of HbA1c is below about 9.0%.

In some embodiments, the individual exhibits normal blood glucose control if the measurement of HbA1c is between 0% and about 7.5%, e.g., about 7.4%, about 7.3%, about 7.2%, about 7.1%, about 7%, about 6.9%, about 6.8%, about 6.7%, about 6.6%, about 6.5%, about 6.4%, about 6.3%, about 6.2%, about 6.1%, about 6%, about 5.9%, about 5.8%, about 5.7%, about 5.6%, about 5.5%, about 5.4%, about 5.3%, about 5.2%, about 5.1%, about 5%, about 4.9%, about 4.8%, about 4.7%, about 4.6%, about 4.5%, about 4.4%, about 4.3%, about 4.2%, about 4.1%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1%, or any percentage therebetween.

In some embodiments, the method of treating an individual afflicted with an IGF1R associated disorder comprises a step of not selecting an individual afflicted with the IGF1R associated disorder for treatment with an antagonist of IGF1R signaling if the individual exhibits abnormal blood glucose control. In some embodiments, the individual exhibits abnormal blood glucose control if the measurement of HbA1c is at least about 4.5%. In some embodiments, the individual exhibits abnormal blood glucose control if the measurement of HbA1c is at least about 4.6%. In some embodiments, the individual exhibits abnormal blood glucose control if the measurement of HbA1c is at least about 4.7%. In some embodiments, the individual exhibits abnormal blood glucose control if the measurement of HbA1c is at least about 4.8%. In some embodiments, the individual exhibits abnormal blood glucose control if the measurement of HbA1c is at least about 4.9%. In some embodiments, the individual exhibits abnormal blood glucose control if the measurement of HbA1c is at least about 5.0%. In some embodiments, the individual exhibits abnormal blood glucose control if the measurement of HbA1c is at least about 5.1%. In some embodiments, the individual exhibits abnormal blood glucose control if the measurement of HbA1c is at least about 5.2%. In some embodiments, the individual exhibits abnormal blood glucose control if the measurement of HbA1c is at least about 5.3%. In some embodiments, the individual exhibits abnormal blood glucose control if the measurement of HbA1c is at least about 5.4%. In some embodiments, the individual exhibits abnormal blood glucose control if the measurement of HbA1c is at least about 5.5%. In some embodiments, the individual exhibits abnormal blood glucose control if the measurement of HbA1c is at least about 5.6%. In some embodiments, the individual exhibits abnormal blood glucose control if the measurement of HbA1c is at least about 5.7%. In some embodiments, the individual exhibits abnormal blood glucose control if the measurement of HbA1c is at least about 5.8%. In some embodiments, the individual exhibits abnormal blood glucose control if the measurement of HbA1c is at least about 5.9%. In some embodiments, the individual exhibits abnormal blood glucose control if the measurement of HbA1c is at least about 6.0%. In some embodiments, the individual exhibits abnormal blood glucose control if the measurement of HbA1c is at least about 6.1%. In some embodiments, the individual exhibits abnormal blood glucose control if the measurement of HbA1c is at least about 6.2%. In some embodiments, the individual exhibits abnormal blood glucose control if the measurement of HbA1c is at least about 6.3%. In some embodiments, the individual exhibits abnormal blood glucose control if the measurement of HbA1c is at least about 6.4%. In some embodiments, the individual exhibits abnormal blood glucose control if the measurement of HbA1c is at least about 6.5%. In some embodiments, the individual exhibits abnormal blood glucose control if the measurement of HbA1c is at least about 6.6%. In some embodiments, the individual exhibits abnormal blood glucose control if the measurement of HbA1c is at least about 6.7%. In some embodiments, the individual exhibits abnormal blood glucose control if the measurement of HbA1c is at least about 6.8%. In some embodiments, the individual exhibits abnormal blood glucose control if the measurement of HbA1c is at least about 6.9%. In some embodiments, the individual exhibits abnormal blood glucose control if the measurement of HbA1c is at least about 7.0%. In some embodiments, the individual exhibits abnormal blood glucose control if the measurement of HbA1c is at least about 7.1%. In some embodiments, the individual exhibits abnormal blood glucose control if the measurement of HbA1c is at least about 7.2%. %. In some embodiments, the individual exhibits abnormal blood glucose control if the measurement of HbA1c is at least about 7.3%. %. In some embodiments, the individual exhibits abnormal blood glucose control if the measurement of HbA1c is at least about 7.4%. %. In some embodiments, the individual exhibits abnormal blood glucose control if the measurement of HbA1c is at least about 7.5%. In some embodiments, the individual exhibits abnormal blood glucose control if the measurement of HbA1c is at least about 7.6%. In some embodiments, the individual exhibits abnormal blood glucose control if the measurement of HbA1c is at least about 7.7%. In some embodiments, the individual exhibits abnormal blood glucose control if the measurement of HbA1c is at least about 7.8%. In some embodiments, the individual exhibits abnormal blood glucose control if the measurement of HbA1c is at least about 7.9%. In some embodiments, the individual exhibits abnormal blood glucose control if the measurement of HbA1c is at least about 8.0%. In some embodiments, the individual exhibits abnormal blood glucose control if the measurement of HbA1c is at least about 8.5%. In some embodiments, the individual exhibits abnormal blood glucose control if the measurement of HbA1c is at least about 9.0%. In some embodiments, the individual exhibits abnormal blood glucose control if the measurement of HbA1c is at least about 4.5%, e.g., about 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, or 8%, or any percentage above 8%. In some embodiments, the test for blood glucose control comprises an oral glucose tolerance test. In some embodiments, the oral glucose tolerance test comprises a measurement of an individual's response to glucose. In some embodiments, the oral glucose tolerance test comprises subjecting an individual to fasting for a period of time. In some embodiments, the period of time for fasting is overnight. In some embodiments, the period of time for fasting is from about 6 hours to about 18 hours, e.g., about 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, or 18 hours, or any duration therebetween. In some embodiments, the period of time for fasting is about 6 hours to about 18 hours, about 6 hours to about 17.5 hours, about 6 hours to about 17 hours, about 6 hours to about 16.5 hours, about 6 hours to about 16 hours, about 6 hours to about 15.5 hours, about 6 hours to about 15 hours, about 6 hours to about 14.5 hours, about 6 hours to about 14 hours, about 6 hours to about 13.5 hours, about 6 hours to about 13 hours, about 6 hours to about 12.5 hours, about 6 hours to about 12 hours, about 6 hours to about 11.5 hours, about 6 hours to about 11 hours, about 6 hours to about 10.5 hours, about 6 hours to about 10 hours, about 6 hours to about 9.5 hours, about 6 hours to about 9 hours, about 6 hours to about 8.5 hours, about 6 hours to about 8 hours, about 6 hours to about 7.5 hours, about 6 hours to about 7 hours, about 6 hours to about 6.5 hours, about 7 hours to about 18 hours, about 7 hours to about 17.5 hours, about 7 hours to about 17 hours, about 7 hours to about 16.5 hours, about 7 hours to about 16 hours, about 7 hours to about 15.5 hours, about 7 hours to about 15 hours, about 7 hours to about 14.5 hours, about 7 hours to about 14 hours, about 7 hours to about 13.5 hours, about 7 hours to about 13 hours, about 7 hours to about 12.5 hours, about 7 hours to about 12 hours, about 7 hours to about 11.5 hours, about 7 hours to about 11 hours, about 7 hours to about 10.5 hours, about 7 hours to about 10 hours, about 7 hours to about 9.5 hours, about 7 hours to about 9 hours, about 7 hours to about 8.5 hours, about 7 hours to about 8 hours, about 7 hours to about 7.5 hours, about 8 hours to about 18 hours, about 8 hours to about 17.5 hours, about 8 hours to about 17 hours, about 8 hours to about 16.5 hours, about 8 hours to about 16 hours, about 8 hours to about 15.5 hours, about 8 hours to about 15 hours, about 8 hours to about 14.5 hours, about 8 hours to about 14 hours, about 8 hours to about 13.5 hours, about 8 hours to about 13 hours, about 8 hours to about 12.5 hours, about 8 hours to about 12 hours, about 8 hours to about 11.5 hours, about 8 hours to about 11 hours, about 8 hours to about 10.5 hours, about 8 hours to about 10 hours, about 8 hours to about 9.5 hours, about 8 hours to about 9 hours, about 8 hours to about 8.5 hours, about 9 hours to about 18 hours, about 9 hours to about 17.5 hours, about 9 hours to about 17 hours, about 9 hours to about 16.5 hours, about 9 hours to about 16 hours, about 9 hours to about 15.5 hours, about 9 hours to about 15 hours, about 9 hours to about 14.5 hours, about 9 hours to about 14 hours, about 9 hours to about 13.5 hours, about 9 hours to about 13 hours, about 9 hours to about 12.5 hours, about 9 hours to about 12 hours, about 9 hours to about 11.5 hours, about 9 hours to about 11 hours, about 9 hours to about 10.5 hours, about 9 hours to about 10 hours, about 9 hours to about 9.5 hours, about 10 hours to about 18 hours, about 10 hours to about 17.5 hours, about 10 hours to about 17 hours, about 10 hours to about 16.5 hours, about 10 hours to about 16 hours, about 10 hours to about 15.5 hours, about 10 hours to about 15 hours, about 10 hours to about 14.5 hours, about 10 hours to about 14 hours, about 10 hours to about 13.5 hours, about 10 hours to about 13 hours, about 10 hours to about 12.5 hours, about 10 hours to about 12 hours, about 10 hours to about 11.5 hours, about 10 hours to about 11 hours, about 10 hours to about 10.5 hours, about 11 hours to about 18 hours, about 11 hours to about 17.5 hours, about 11 hours to about 17 hours, about 11 hours to about 16.5 hours, about 11 hours to about 16 hours, about 11 hours to about 15.5 hours, about 11 hours to about 15 hours, about 11 hours to about 14.5 hours, about 11 hours to about 14 hours, about 11 hours to about 13.5 hours, about 11 hours to about 13 hours, about 11 hours to about 12.5 hours, about 11 hours to about 12 hours, about 11 hours to about 11.5 hours, about 12 hours to about 18 hours, about 12 hours to about 17.5 hours, about 12 hours to about 17 hours, about 12 hours to about 16.5 hours, about 12 hours to about 16 hours, about 12 hours to about 15.5 hours, about 12 hours to about 15 hours, about 12 hours to about 14.5 hours, about 12 hours to about 14 hours, about 12 hours to about 13.5 hours, about 12 hours to about 13 hours, about 12 hours to about 12.5 hours, about 13 hours to about 18 hours, about 13 hours to about 17.5 hours, about 13 hours to about 17 hours, about 13 hours to about 16.5 hours, about 13 hours to about 16 hours, about 13 hours to about 15.5 hours, about 13 hours to about 15 hours, about 13 hours to about 14.5 hours, about 13 hours to about 14 hours, about 13 hours to about 13.5 hours, about 14 hours to about 18 hours, about 14 hours to about 17.5 hours, about 14 hours to about 17 hours, about 14 hours to about 16.5 hours, about 14 hours to about 16 hours, about 14 hours to about 15.5 hours, about 14 hours to about 15 hours, about 14 hours to about 14.5 hours, about 15 hours to about 18 hours, about 15 hours to about 17.5 hours, about 15 hours to about 17 hours, about 15 hours to about 16.5 hours, about 15 hours to about 16 hours, about 15 hours to about 15.5 hours, about 16 hours to about 18 hours, about 16 hours to about 17.5 hours, about 16 hours to about 17 hours, about 16 hours to about 16.5 hours, about 17 hours to about 18 hours, or about 17 hours to about 17.5 hours.

In some embodiments, the oral glucose tolerance test comprises measuring a first blood glucose level of the individual after fasting. In some embodiments, the oral glucose tolerance test comprises the individual orally ingesting an amount of sugar after fasting. In some embodiments, the oral glucose tolerance test comprises the individual orally ingesting an amount of sugar after the measuring the first blood glucose level of the individual. In some embodiments, the oral glucose tolerance test comprises the individual orally ingesting an amount of glucose after fasting. In some embodiments, the oral glucose tolerance test comprises the individual orally ingesting an amount of glucose after the measuring the first blood glucose level of the individual. In some embodiments, the amount of sugar or glucose being ingested is between about 50 to 150 grams (g), e.g., about 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g, 112 g, 113 g, 114 g, 115 g, 116 g, 117 g, 118 g, 119 g, 120 g, 121 g, 122 g, 123 g, 124 g, 125 g, 126 g, 127 g, 128 g, 129 g, 130 g, 131 g, 132 g, 133 g, 134 g, 135 g, 136 g, 137 g, 138 g, 139 g, 140 g, 141 g, 142 g, 143 g, 144 g, 145 g, 146 g, 147 g, 148 g, 149 g, or about 150 g, or any amount therebetween. In some embodiments, the amount of sugar being ingested is about 75 g. In some embodiments, the amount of glucose being ingested is about 75 g. In some embodiments, the oral glucose tolerance test comprises the individual orally ingesting about 75 g of sugar dissolved in an aqueous medium after fasting. In some embodiments, the oral glucose tolerance test comprises the individual orally ingesting about 75 g of glucose dissolved in an aqueous medium after fasting. In some embodiments, the oral glucose tolerance test comprises the individual orally ingesting about 75 g of sugar dissolved in an aqueous medium after the measuring the first blood glucose level of the individual. In some embodiments, the oral glucose tolerance test comprises the individual orally ingesting about 75 g of glucose dissolved in an aqueous medium after the measuring the first blood glucose level of the individual. In some embodiments, the aqueous medium comprises water.

In some embodiments, the oral glucose tolerance test comprises measuring a second blood glucose level of the individual after the individual's oral ingestion of sugar or glucose. In some embodiments, the time duration between the individual's oral ingestion of sugar or glucose and the measuring the second blood glucose level of the individual is about 30 minutes (min) to about 6 hours, e.g., about 30 min, 1 hour, 1.5 hours, 2 hours, 2.5 hours, 3 hours, 3.5 hours, 4 hours, 4.5 hours, 5 hours, 5.5 hours, or about 6 hours, or any duration therebetween. In some embodiments, the time duration between the individual's oral ingestion of about 75 g of sugar or glucose dissolved in an aqueous medium and the measuring the second blood glucose level of the individual is about 30 minutes (min) to about 6 hours, e.g., about 30 min, 1 hour, 1.5 hours, 2 hours, 2.5 hours, 3 hours, 3.5 hours, 4 hours, 4.5 hours, 5 hours, 5.5 hours, or about 6 hours, or any duration therebetween. In some embodiments, the time duration between the individual's oral ingestion of sugar or glucose dissolved in an aqueous medium and the measuring the second blood glucose level of the individual is about 30 min to about 6 hours, about 30 min to about 5.5 hours, about 30 min to about 5 hours, about 30 min to about 4.5 hours, about 30 min to about 4 hours, about 30 min to about 3.5 hours, about 30 min to about 3 hours, about 30 min to about 2.5 hours, about 30 min to about 2 hours, about 30 min to about 1.5 hours, about 30 min to about 1 hour, about 1 hour to about 6 hours, about 1 hour to about 5.5 hours, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours, about 2 hours to about 6 hours, about 2 hours to about 5.5 hours, about 2 hours to about 5 hours, about 2 hours to about 4.5 hours, about 2 hours to about 4 hours, about 2 hours to about 3.5 hours, about 2 hours to about 3 hours, about 2 hours to about 2.5 hours, about 2.5 hours to about 6 hours, about 2.5 hours to about 5.5 hours, about 2.5 hours to about 5 hours, about 2.5 hours to about 4.5 hours, about 2.5 hours to about 4 hours, about 2.5 hours to about 3.5 hours, about 2.5 hours to about 3 hours, about 3 hours to about 6 hours, about 3 hours to about 5.5 hours, about 3 hours to about 5 hours, about 3 hours to about 4.5 hours, about 3 hours to about 4 hours, about 3 hours to about 3.5 hours, about 4 hours to about 6 hours, about 4 hours to about 5.5 hours, about 4 hours to about 5 hours, or about 4 hours to about 4.5 hours. In some embodiments, the time duration between the individual's oral ingestion of 75 g of sugar or glucose dissolved in an aqueous medium and the measuring the second blood glucose level of the individual is about 30 min to about 6 hours, about 30 min to about 5.5 hours, about 30 min to about 5 hours, about 30 min to about 4.5 hours, about 30 min to about 4 hours, about 30 min to about 3.5 hours, about 30 min to about 3 hours, about 30 min to about 2.5 hours, about 30 min to about 2 hours, about 30 min to about 1.5 hours, about 30 min to about 1 hour, about 1 hour to about 6 hours, about 1 hour to about 5.5 hours, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours, about 1.5 hours to about 6 hours, about 1.5 hours to about 5.5 hours, about 1.5 hours to about 5 hours, about 1.5 hours to about 4.5 hours, about 1.5 hours to about 4 hours, about 1.5 hours to about 3.5 hours, about 1.5 hours to about 3 hours, about 1.5 hours to about 2.5 hours, about 1.5 hours to about 2 hours, about 2 hours to about 6 hours, about 2 hours to about 5.5 hours, about 2 hours to about 5 hours, about 2 hours to about 4.5 hours, about 2 hours to about 4 hours, about 2 hours to about 3.5 hours, about 2 hours to about 3 hours, about 2 hours to about 2.5 hours, about 2.5 hours to about 6 hours, about 2.5 hours to about 5.5 hours, about 2.5 hours to about 5 hours, about 2.5 hours to about 4.5 hours, about 2.5 hours to about 4 hours, about 2.5 hours to about 3.5 hours, about 2.5 hours to about 3 hours, about 3 hours to about 6 hours, about 3 hours to about 5.5 hours, about 3 hours to about 5 hours, about 3 hours to about 4.5 hours, about 3 hours to about 4 hours, about 3 hours to about 3.5 hours, about 3.5 hours to about 6 hours, about 3.5 hours to about 5.5 hours, about 3.5 hours to about 5 hours, about 3.5 hours to about 4.5 hours, about 3.5 hours to about 4 hours, about 4 hours to about 6 hours, about 4 hours to about 5.5 hours, about 4 hours to about 5 hours, about 4 hours to about 4.5 hours, about 4.5 hours to about 6 hours, about 4.5 hours to about 5.5 hours, about 4.5 hours to about 5 hours, about 5 hours to about 6 hours, or about 5.5 hours to about 6 hours.

In some embodiments, the time duration between the individual's oral ingestion of 75 g of sugar or glucose dissolved in an aqueous medium and the measuring the second blood glucose level of the individual is about 1 hour. In some embodiments, the time duration between the individual's oral ingestion of about 75 g of sugar or glucose dissolved in an aqueous medium and the measuring the second blood glucose level of the individual is about 1.5 hours. In some embodiments, the time duration between the individual's oral ingestion of about 75 g of sugar or glucose dissolved in an aqueous medium and the measuring the second blood glucose level of the individual is about 2 hours. In some embodiments, the time duration between the individual's oral ingestion of about 75 g of sugar or glucose dissolved in an aqueous medium and the measuring the second blood glucose level of the individual is about 2.5 hours. In some embodiments, the time duration between the individual's oral ingestion of about 75 g of sugar or glucose dissolved in an aqueous medium and the measuring the second blood glucose level of the individual is about 3 hours. In some embodiments, the time duration between the individual's oral ingestion of about 75 g of sugar or glucose dissolved in an aqueous medium and the measuring the second blood glucose level of the individual is about 3.5 hours. In some embodiments, the time duration between the individual's oral ingestion of about 75 g of sugar or glucose dissolved in an aqueous medium and the measuring the second blood glucose level of the individual is about 4 hours.

In some embodiments, the individual exhibits normal blood glucose control if the second blood glucose level of the individual measured by the oral glucose tolerance test is no more than 250 milligrams per deciliter (mg/dL). In some embodiments, the individual exhibits normal blood glucose control if the second blood glucose level of the individual measured by the oral glucose tolerance test is no more than 245 mg/dL. In some embodiments, the individual exhibits normal blood glucose control if the second blood glucose level of the individual measured by the oral glucose tolerance test is no more than 240 mg/dL. In some embodiments, the individual exhibits normal blood glucose control if the second blood glucose level of the individual measured by the oral glucose tolerance test is no more than 235 mg/dL. In some embodiments, the individual exhibits normal blood glucose control if the second blood glucose level of the individual measured by the oral glucose tolerance test is no more than 230 mg/dL. In some embodiments, the individual exhibits normal blood glucose control if the second blood glucose level of the individual measured by the oral glucose tolerance test is no more than 225 mg/dL. In some embodiments, the individual exhibits normal blood glucose control if the second blood glucose level of the individual measured by the oral glucose tolerance test is no more than 220 mg/dL. In some embodiments, the individual exhibits normal blood glucose control if the second blood glucose level of the individual measured by the oral glucose tolerance test is no more than 215 mg/dL. In some embodiments, the individual exhibits normal blood glucose control if the second blood glucose level of the individual measured by the oral glucose tolerance test is no more than 210 mg/dL. In some embodiments, the individual exhibits normal blood glucose control if the second blood glucose level of the individual measured by the oral glucose tolerance test is no more than 205 mg/dL. In some embodiments, the individual exhibits normal blood glucose control if the second blood glucose level of the individual measured by the oral glucose tolerance test is no more than 200 mg/dL. In some embodiments, the individual exhibits normal blood glucose control if the second blood glucose level of the individual measured by the oral glucose tolerance test is no more than 195 mg/dL. In some embodiments, the individual exhibits normal blood glucose control if the second blood glucose level of the individual measured by the oral glucose tolerance test is no more than 190 mg/dL. In some embodiments, the individual exhibits normal blood glucose control if the second blood glucose level of the individual measured by the oral glucose tolerance test is no more than 185 mg/dL. In some embodiments, the individual exhibits normal blood glucose control if the second blood glucose level of the individual measured by the oral glucose tolerance test is no more than 180 mg/dL. In some embodiments, the individual exhibits normal blood glucose control if the second blood glucose level of the individual measured by the oral glucose tolerance test is no more than 175 mg/dL. In some embodiments, the individual exhibits normal blood glucose control if the second blood glucose level of the individual measured by the oral glucose tolerance test is no more than 170 mg/dL. In some embodiments, the individual exhibits normal blood glucose control if the second blood glucose level of the individual measured by the oral glucose tolerance test is no more than 165 mg/dL. In some embodiments, the individual exhibits normal blood glucose control if the second blood glucose level of the individual measured by the oral glucose tolerance test is no more than 160 mg/dL. In some embodiments, the individual exhibits normal blood glucose control if the second blood glucose level of the individual measured by the oral glucose tolerance test is no more than 155 mg/dL. In some embodiments, the individual exhibits normal blood glucose control if the second blood glucose level of the individual measured by the oral glucose tolerance test is no more than 150 mg/dL. In some embodiments, the individual exhibits normal blood glucose control if the second blood glucose level of the individual measured by the oral glucose tolerance test is no more than 145 mg/dL. In some embodiments, the individual exhibits normal blood glucose control if the second blood glucose level of the individual measured by the oral glucose tolerance test is no more than 140 mg/dL. In some embodiments, the individual exhibits normal blood glucose control if the second blood glucose level of the individual measured by the oral glucose tolerance test is no more than 135 mg/dL. In some embodiments, the individual exhibits normal blood glucose control if the second blood glucose level of the individual measured by the oral glucose tolerance test is no more than 130 mg/dL. In some embodiments, the individual exhibits normal blood glucose control if the second blood glucose level of the individual measured by the oral glucose tolerance test is no more than 125 mg/dL. In some embodiments, the individual exhibits normal blood glucose control if the second blood glucose level of the individual measured by the oral glucose tolerance test is no more than 120 mg/dL. In some embodiments, the individual exhibits normal blood glucose control if the second blood glucose level of the individual measured by the oral glucose tolerance test is no more than 115 mg/dL. In some embodiments, the individual exhibits normal blood glucose control if the second blood glucose level of the individual measured by the oral glucose tolerance test is no more than 110 mg/dL. In some embodiments, the individual exhibits normal blood glucose control if the second blood glucose level of the individual measured by the oral glucose tolerance test is no more than 105 mg/dL. In some embodiments, the individual exhibits normal blood glucose control if the second blood glucose level of the individual measured by the oral glucose tolerance test is no more than 100 mg/dL.

In some embodiments, the individual exhibits normal blood glucose control if the second blood glucose level of the individual measured by the oral glucose tolerance test is no more than 250 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 245 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 240 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 235 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 230 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 225 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 220 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 215 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 210 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 205 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 200 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 195 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 190 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 185 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 180 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 175 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 170 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 165 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 160 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 155 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 150 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 145 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 140 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 135 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 130 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 125 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 120 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 115 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 110 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 105 mg/dL and the second blood glucose level is measured about 2 hours after the individual's oral ingestion of about 75 g of glucose or sugar dissolved in an aqueous medium. In some embodiments, the individual exhibits normal blood glucose control if the second blood glucose level of the individual measured by the oral glucose tolerance test is no more than 100 mg/dL and the second blood glucose level is measured about 2 hours after the individual's oral ingestion of about 75 g of glucose or sugar dissolved in an aqueous medium.

In some embodiments, the individual exhibits normal blood glucose control if the second blood glucose level of the individual measured by the oral glucose tolerance test is about 0 to about 250 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, or 250 mg/dL, or any concentration therebetween.

In some embodiments, during the oral glucose tolerance test, when the second blood glucose level is measured 2 hours after the individual's oral ingestion of 75 g of glucose or sugar dissolved in an aqueous medium, the individual exhibits normal blood glucose control if the second blood glucose level is between about 0 and about 250 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, or 250 mg/dL, or any concentration therebetween.

In some embodiments, the individual exhibits abnormal blood glucose control if the second blood glucose level of the individual measured by the oral glucose tolerance test is above 250 mg/dL. In some embodiments, the individual exhibits abnormal blood glucose control if the second blood glucose level of the individual measured by the oral glucose tolerance test is above 240 mg/dL. In some embodiments, the individual exhibits abnormal blood glucose control if the second blood glucose level of the individual measured by the oral glucose tolerance test is above 230 mg/dL. In some embodiments, the individual exhibits abnormal blood glucose control if the second blood glucose level of the individual measured by the oral glucose tolerance test is above 220 mg/dL. In some embodiments, the individual exhibits abnormal blood glucose control if the second blood glucose level of the individual measured by the oral glucose tolerance test is above 210 mg/dL. In some embodiments, the individual exhibits abnormal blood glucose control if the second blood glucose level of the individual measured by the oral glucose tolerance test is above 200 mg/dL. In some embodiments, the individual exhibits abnormal blood glucose control if the second blood glucose level of the individual measured by the oral glucose tolerance test is above 190 mg/dL. In some embodiments, the individual exhibits abnormal blood glucose control if the second blood glucose level of the individual measured by the oral glucose tolerance test is above 180 mg/dL. In some embodiments, the individual exhibits abnormal blood glucose control if the second blood glucose level of the individual measured by the oral glucose tolerance test is above 170 mg/dL. In some embodiments, the individual exhibits abnormal blood glucose control if the second blood glucose level of the individual measured by the oral glucose tolerance test is above 160 mg/dL. In some embodiments, the individual exhibits abnormal blood glucose control if the second blood glucose level of the individual measured by the oral glucose tolerance test is above 150 mg/dL. In some embodiments, the individual exhibits abnormal blood glucose control if the second blood glucose level of the individual measured by the oral glucose tolerance test is above 140 mg/dL. In some embodiments, the individual exhibits abnormal blood glucose control if the second blood glucose level of the individual measured by the oral glucose tolerance test is above 130 mg/dL. In some embodiments, the individual exhibits abnormal blood glucose control if the second blood glucose level of the individual measured by the oral glucose tolerance test is above 120 mg/dL. In some embodiments, the individual exhibits abnormal blood glucose control if the second blood glucose level of the individual measured by the oral glucose tolerance test is above 110 mg/dL. In some embodiments, the individual exhibits abnormal blood glucose control if the second blood glucose level of the individual measured by the oral glucose tolerance test is above 100 mg/dL.

In some embodiments, the individual exhibits abnormal blood glucose control if the second blood glucose level of the individual measured by the oral glucose tolerance test is above 250 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 240 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 230 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 220 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 210 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 200 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 190 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 180 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 170 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 160 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 150 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 140 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 130 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 120 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 110 mg/dL and the second blood glucose level is measured 2 hours after the individual's oral ingestion of 75 g of glucose or sugar dissolved in an aqueous medium. In some embodiments, the individual exhibits abnormal blood glucose control if the second blood glucose level of the individual measured by the oral glucose tolerance test is above 100 mg/dL and the second blood glucose level is measured 2 hours after the individual's oral ingestion of 75 g of glucose or sugar dissolved in an aqueous medium.

In some embodiments, the individual exhibits abnormal blood glucose control if the second blood glucose level of the individual measured by the oral glucose tolerance test is above about 100 mg/dL, e.g., about 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, 251 mg/dL, 252 mg/dL, 253 mg/dL, 254 mg/dL, 255 mg/dL, 256 mg/dL, 257 mg/dL, 258 mg/dL, 259 mg/dL, 260 mg/dL, 261 mg/dL, 262 mg/dL, 263 mg/dL, 264 mg/dL, 265 mg/dL, 266 mg/dL, 267 mg/dL, 268 mg/dL, 269 mg/dL, 270 mg/dL, 271 mg/dL, 272 mg/dL, 273 mg/dL, 274 mg/dL, or about 275 mg/dL, or any concentration above about 275 mg/dL.

In some embodiments, during the oral glucose tolerance test, when the second blood glucose level is measured 2 hours after the individual's oral ingestion of 75 g of glucose or sugar dissolved in an aqueous medium, the individual exhibits abnormal blood glucose control if the second blood glucose level is above about 100 mg/dL, e.g., about 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, 251 mg/dL, 252 mg/dL, 253 mg/dL, 254 mg/dL, 255 mg/dL, 256 mg/dL, 257 mg/dL, 258 mg/dL, 259 mg/dL, 260 mg/dL, 261 mg/dL, 262 mg/dL, 263 mg/dL, 264 mg/dL, 265 mg/dL, 266 mg/dL, 267 mg/dL, 268 mg/dL, 269 mg/dL, 270 mg/dL, 271 mg/dL, 272 mg/dL, 273 mg/dL, 274 mg/dL, or about 275 mg/dL, or any concentration above about 275 mg/dL.

In some embodiments, the test for blood glucose control comprises a measurement of fasting blood glucose. In some embodiments, the measurement of fasting blood glucose comprises subjecting an individual to fasting. In some embodiments, the fasting lasts overnight. In some embodiments, the fasting lasts about 6 hours to about 18 hours, e.g., about 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, or 18 hours, or any duration therebetween. In some embodiments, the fasting lasts about 6 hours to about 18 hours, about 6 hours to about 17.5 hours, about 6 hours to about 17 hours, about 6 hours to about 16.5 hours, about 6 hours to about 16 hours, about 6 hours to about 15.5 hours, about 6 hours to about 15 hours, about 6 hours to about 14.5 hours, about 6 hours to about 14 hours, about 6 hours to about 13.5 hours, about 6 hours to about 13 hours, about 6 hours to about 12.5 hours, about 6 hours to about 12 hours, about 6 hours to about 11.5 hours, about 6 hours to about 11 hours, about 6 hours to about 10.5 hours, about 6 hours to about 10 hours, about 6 hours to about 9.5 hours, about 6 hours to about 9 hours, about 6 hours to about 8.5 hours, about 6 hours to about 8 hours, about 6 hours to about 7.5 hours, about 6 hours to about 7 hours, about 6 hours to about 6.5 hours, about 7 hours to about 18 hours, about 7 hours to about 17.5 hours, about 7 hours to about 17 hours, about 7 hours to about 16.5 hours, about 7 hours to about 16 hours, about 7 hours to about 15.5 hours, about 7 hours to about 15 hours, about 7 hours to about 14.5 hours, about 7 hours to about 14 hours, about 7 hours to about 13.5 hours, about 7 hours to about 13 hours, about 7 hours to about 12.5 hours, about 7 hours to about 12 hours, about 7 hours to about 11.5 hours, about 7 hours to about 11 hours, about 7 hours to about 10.5 hours, about 7 hours to about 10 hours, about 7 hours to about 9.5 hours, about 7 hours to about 9 hours, about 7 hours to about 8.5 hours, about 7 hours to about 8 hours, about 7 hours to about 7.5 hours, about 8 hours to about 18 hours, about 8 hours to about 17.5 hours, about 8 hours to about 17 hours, about 8 hours to about 16.5 hours, about 8 hours to about 16 hours, about 8 hours to about 15.5 hours, about 8 hours to about 15 hours, about 8 hours to about 14.5 hours, about 8 hours to about 14 hours, about 8 hours to about 13.5 hours, about 8 hours to about 13 hours, about 8 hours to about 12.5 hours, about 8 hours to about 12 hours, about 8 hours to about 11.5 hours, about 8 hours to about 11 hours, about 8 hours to about 10.5 hours, about 8 hours to about 10 hours, about 8 hours to about 9.5 hours, about 8 hours to about 9 hours, about 8 hours to about 8.5 hours, about 9 hours to about 18 hours, about 9 hours to about 17.5 hours, about 9 hours to about 17 hours, about 9 hours to about 16.5 hours, about 9 hours to about 16 hours, about 9 hours to about 15.5 hours, about 9 hours to about 15 hours, about 9 hours to about 14.5 hours, about 9 hours to about 14 hours, about 9 hours to about 13.5 hours, about 9 hours to about 13 hours, about 9 hours to about 12.5 hours, about 9 hours to about 12 hours, about 9 hours to about 11.5 hours, about 9 hours to about 11 hours, about 9 hours to about 10.5 hours, about 9 hours to about 10 hours, about 9 hours to about 9.5 hours, about 10 hours to about 18 hours, about 10 hours to about 17.5 hours, about 10 hours to about 17 hours, about 10 hours to about 16.5 hours, about 10 hours to about 16 hours, about 10 hours to about 15.5 hours, about 10 hours to about 15 hours, about 10 hours to about 14.5 hours, about 10 hours to about 14 hours, about 10 hours to about 13.5 hours, about 10 hours to about 13 hours, about 10 hours to about 12.5 hours, about 10 hours to about 12 hours, about 10 hours to about 11.5 hours, about 10 hours to about 11 hours, about 10 hours to about 10.5 hours, about 11 hours to about 18 hours, about 11 hours to about 17.5 hours, about 11 hours to about 17 hours, about 11 hours to about 16.5 hours, about 11 hours to about 16 hours, about 11 hours to about 15.5 hours, about 11 hours to about 15 hours, about 11 hours to about 14.5 hours, about 11 hours to about 14 hours, about 11 hours to about 13.5 hours, about 11 hours to about 13 hours, about 11 hours to about 12.5 hours, about 11 hours to about 12 hours, about 11 hours to about 11.5 hours, about 12 hours to about 18 hours, about 12 hours to about 17.5 hours, about 12 hours to about 17 hours, about 12 hours to about 16.5 hours, about 12 hours to about 16 hours, about 12 hours to about 15.5 hours, about 12 hours to about 15 hours, about 12 hours to about 14.5 hours, about 12 hours to about 14 hours, about 12 hours to about 13.5 hours, about 12 hours to about 13 hours, about 12 hours to about 12.5 hours, about 13 hours to about 18 hours, about 13 hours to about 17.5 hours, about 13 hours to about 17 hours, about 13 hours to about 16.5 hours, about 13 hours to about 16 hours, about 13 hours to about 15.5 hours, about 13 hours to about 15 hours, about 13 hours to about 14.5 hours, about 13 hours to about 14 hours, about 13 hours to about 13.5 hours, about 14 hours to about 18 hours, about 14 hours to about 17.5 hours, about 14 hours to about 17 hours, about 14 hours to about 16.5 hours, about 14 hours to about 16 hours, about 14 hours to about 15.5 hours, about 14 hours to about 15 hours, about 14 hours to about 14.5 hours, about 15 hours to about 18 hours, about 15 hours to about 17.5 hours, about 15 hours to about 17 hours, about 15 hours to about 16.5 hours, about 15 hours to about 16 hours, about 15 hours to about 15.5 hours, about 16 hours to about 18 hours, about 16 hours to about 17.5 hours, about 16 hours to about 17 hours, about 16 hours to about 16.5 hours, about 17 hours to about 18 hours, or about 17 hours to about 17.5 hours.

In some embodiments, the measurement of fasting blood glucose test comprises measuring a blood glucose level of the individual after the fast. In some embodiments, the individual exhibits normal blood glucose control if the blood glucose level of the individual after the fast is no more than about 150 mg/dL. In some embodiments, the individual exhibits normal blood glucose control if the blood glucose level of the individual after the fast is no more than about 149 mg/dL. In some embodiments, the individual exhibits normal blood glucose control if the blood glucose level of the individual after the fast is no more than about 148 mg/dL. In some embodiments, the individual exhibits normal blood glucose control if the blood glucose level of the individual after the fast is no more than about 147 mg/dL. In some embodiments, the individual exhibits normal blood glucose control if the blood glucose level of the individual after the fast is no more than about 146 mg/dL. In some embodiments, the individual exhibits normal blood glucose control if the blood glucose level of the individual after the fast is no more than about 145 mg/dL. In some embodiments, the individual exhibits normal blood glucose control if the blood glucose level of the individual after the fast is no more than about 144 mg/dL. In some embodiments, the individual exhibits normal blood glucose control if the blood glucose level of the individual after the fast is no more than about 143 mg/dL. In some embodiments, the individual exhibits normal blood glucose control if the blood glucose level of the individual after the fast is no more than about 142 mg/dL. In some embodiments, the individual exhibits normal blood glucose control if the blood glucose level of the individual after the fast is no more than about 141 mg/dL. In some embodiments, the individual exhibits normal blood glucose control if the blood glucose level of the individual after the fast is no more than about 140 mg/dL. In some embodiments, the individual exhibits normal blood glucose control if the blood glucose level of the individual after the fast is no more than about 139 mg/dL. In some embodiments, the individual exhibits normal blood glucose control if the blood glucose level of the individual after the fast is no more than about 138 mg/dL. In some embodiments, the individual exhibits normal blood glucose control if the blood glucose level of the individual after the fast is no more than about 137 mg/dL. In some embodiments, the individual exhibits normal blood glucose control if the blood glucose level of the individual after the fast is no more than about 136 mg/dL. In some embodiments, the individual exhibits normal blood glucose control if the blood glucose level of the individual after the fast is no more than about 135 mg/dL. In some embodiments, the individual exhibits normal blood glucose control if the blood glucose level of the individual after the fast is no more than about 134 mg/dL. In some embodiments, the individual exhibits normal blood glucose control if the blood glucose level of the individual after the fast is no more than about 133 mg/dL. In some embodiments, the individual exhibits normal blood glucose control if the blood glucose level of the individual after the fast is no more than about 132 mg/dL. In some embodiments, the individual exhibits normal blood glucose control if the blood glucose level of the individual after the fast is no more than about 131 mg/dL. In some embodiments, the individual exhibits normal blood glucose control if the blood glucose level of the individual after the fast is no more than about 130 mg/dL. In some embodiments, the individual exhibits normal blood glucose control if the blood glucose level of the individual after the fast is no more than about 129 mg/dL. In some embodiments, the individual exhibits normal blood glucose control if the blood glucose level of the individual after the fast is no more than about 128 mg/dL. In some embodiments, the individual exhibits normal blood glucose control if the blood glucose level of the individual after the fast is no more than about 127 mg/dL. In some embodiments, the individual exhibits normal blood glucose control if the blood glucose level of the individual after the fast is no more than about 126 mg/dL. In some embodiments, the individual exhibits normal blood glucose control if the blood glucose level of the individual after the fast is no more than about 125 mg/dL. In some embodiments, the individual exhibits normal blood glucose control if the blood glucose level of the individual after the fast is no more than about 124 mg/dL. In some embodiments, the individual exhibits normal blood glucose control if the blood glucose level of the individual after the fast is no more than about 123 mg/dL. In some embodiments, the individual exhibits normal blood glucose control if the blood glucose level of the individual after the fast is no more than about 122 mg/dL. In some embodiments, the individual exhibits normal blood glucose control if the blood glucose level of the individual after the fast is no more than about 121 mg/dL. In some embodiments, the individual exhibits normal blood glucose control if the blood glucose level of the individual after the fast is no more than about 120 mg/dL. In some embodiments, the individual exhibits normal blood glucose control if the blood glucose level of the individual after the fast is no more than about 119 mg/dL. In some embodiments, the individual exhibits normal blood glucose control if the blood glucose level of the individual after the fast is no more than about 118 mg/dL. In some embodiments, the individual exhibits normal blood glucose control if the blood glucose level of the individual after the fast is no more than about 117 mg/dL. In some embodiments, the individual exhibits normal blood glucose control if the blood glucose level of the individual after the fast is no more than about 116 mg/dL. In some embodiments, the individual exhibits normal blood glucose control if the blood glucose level of the individual after the fast is no more than about 115 mg/dL. In some embodiments, the individual exhibits normal blood glucose control if the blood glucose level of the individual after the fast is no more than about 114 mg/dL. In some embodiments, the individual exhibits normal blood glucose control if the blood glucose level of the individual after the fast is no more than about 113 mg/dL. In some embodiments, the individual exhibits normal blood glucose control if the blood glucose level of the individual after the fast is no more than about 112 mg/dL. In some embodiments, the individual exhibits normal blood glucose control if the blood glucose level of the individual after the fast is no more than about 111 mg/dL. In some embodiments, the individual exhibits normal blood glucose control if the blood glucose level of the individual after the fast is no more than about 110 mg/dL. In some embodiments, the individual exhibits normal blood glucose control if the blood glucose level of the individual after the fast is no more than about 109 mg/dL. In some embodiments, the individual exhibits normal blood glucose control if the blood glucose level of the individual after the fast is no more than about 108 mg/dL. In some embodiments, the individual exhibits normal blood glucose control if the blood glucose level of the individual after the fast is no more than about 107 mg/dL. In some embodiments, the individual exhibits normal blood glucose control if the blood glucose level of the individual after the fast is no more than about 106 mg/dL. In some embodiments, the individual exhibits normal blood glucose control if the blood glucose level of the individual after the fast is no more than about 105 mg/dL. In some embodiments, the individual exhibits normal blood glucose control if the blood glucose level of the individual after the fast is no more than about 104 mg/dL. In some embodiments, the individual exhibits normal blood glucose control if the blood glucose level of the individual after the fast is no more than about 103 mg/dL. In some embodiments, the individual exhibits normal blood glucose control if the blood glucose level of the individual after the fast is no more than about 102 mg/dL. In some embodiments, the individual exhibits normal blood glucose control if the blood glucose level of the individual after the fast is no more than about 101 mg/dL. In some embodiments, the individual exhibits normal blood glucose control if the blood glucose level of the individual after the fast is no more than about 100 mg/dL. In some embodiments, the individual exhibits normal blood glucose control if the blood glucose level of the individual after the fast is no more than about 99 mg/dL. In some embodiments, the individual exhibits normal blood glucose control if the blood glucose level of the individual after the fast is no more than about 98 mg/dL. In some embodiments, the individual exhibits normal blood glucose control if the blood glucose level of the individual after the fast is no more than about 97 mg/dL. In some embodiments, the individual exhibits normal blood glucose control if the blood glucose level of the individual after the fast is no more than about 96 mg/dL. In some embodiments, the individual exhibits normal blood glucose control if the blood glucose level of the individual after the fast is no more than about 95 mg/dL. In some embodiments, the individual exhibits normal blood glucose control if the blood glucose level of the individual after the fast is no more than about 94 mg/dL. In some embodiments, the individual exhibits normal blood glucose control if the blood glucose level of the individual after the fast is no more than about 93 mg/dL. In some embodiments, the individual exhibits normal blood glucose control if the blood glucose level of the individual after the fast is no more than about 92 mg/dL. In some embodiments, the individual exhibits normal blood glucose control if the blood glucose level of the individual after the fast is no more than about 91 mg/dL. In some embodiments, the individual exhibits normal blood glucose control if the blood glucose level of the individual after the fast is no more than about 90 mg/dL. In some embodiments, the individual exhibits normal blood glucose control if the blood glucose level of the individual after the fast is no more than about 89 mg/dL. In some embodiments, the individual exhibits normal blood glucose control if the blood glucose level of the individual after the fast is no more than about 88 mg/dL. In some embodiments, the individual exhibits normal blood glucose control if the blood glucose level of the individual after the fast is no more than about 87 mg/dL. In some embodiments, the individual exhibits normal blood glucose control if the blood glucose level of the individual after the fast is no more than about 86 mg/dL. In some embodiments, the individual exhibits normal blood glucose control if the blood glucose level of the individual after the fast is no more than about 85 mg/dL. In some embodiments, the individual exhibits normal blood glucose control if the blood glucose level of the individual after the fast is no more than about 84 mg/dL. In some embodiments, the individual exhibits normal blood glucose control if the blood glucose level of the individual after the fast is no more than about 83 mg/dL. In some embodiments, the individual exhibits normal blood glucose control if the blood glucose level of the individual after the fast is no more than about 82 mg/dL. In some embodiments, the individual exhibits normal blood glucose control if the blood glucose level of the individual after the fast is no more than about 81 mg/dL. In some embodiments, the individual exhibits normal blood glucose control if the blood glucose level of the individual after the fast is no more than about 80 mg/dL. In some embodiments, the individual exhibits normal blood glucose control if the blood glucose level of the individual after the fast is no more than about 79 mg/dL. In some embodiments, the individual exhibits normal blood glucose control if the blood glucose level of the individual after the fast is no more than about 78 mg/dL. In some embodiments, the individual exhibits normal blood glucose control if the blood glucose level of the individual after the fast is no more than about 77 mg/dL. In some embodiments, the individual exhibits normal blood glucose control if the blood glucose level of the individual after the fast is no more than about 76 mg/dL. In some embodiments, the individual exhibits normal blood glucose control if the blood glucose level of the individual after the fast is no more than about 75 mg/dL.

In some embodiments, the individual exhibits normal blood glucose control if the blood glucose level of the individual after the fast is between about 0 and about 150 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or about 150 mg/dL, or any concentration therebetween.

In some embodiments, the individual exhibits normal blood glucose control if the blood glucose level of the individual after the overnight fast is no more than about 150 mg/dL. In some embodiments, the individual exhibits normal blood glucose control if the blood glucose level of the individual after the overnight fast is no more than about 149 mg/dL. In some embodiments, the individual exhibits normal blood glucose control if the blood glucose level of the individual after the overnight fast is no more than about 148 mg/dL. In some embodiments, the individual exhibits normal blood glucose control if the blood glucose level of the individual after the overnight fast is no more than about 147 mg/dL. In some embodiments, the individual exhibits normal blood glucose control if the blood glucose level of the individual after the overnight fast is no more than about 146 mg/dL. In some embodiments, the individual exhibits normal blood glucose control if the blood glucose level of the individual after the overnight fast is no more than about 145 mg/dL. In some embodiments, the individual exhibits normal blood glucose control if the blood glucose level of the individual after the overnight fast is no more than about 144 mg/dL. In some embodiments, the individual exhibits normal blood glucose control if the blood glucose level of the individual after the overnight fast is no more than about 143 mg/dL. In some embodiments, the individual exhibits normal blood glucose control if the blood glucose level of the individual after the overnight fast is no more than about 142 mg/dL. In some embodiments, the individual exhibits normal blood glucose control if the blood glucose level of the individual after the overnight fast is no more than about 141 mg/dL. In some embodiments, the individual exhibits normal blood glucose control if the blood glucose level of the individual after the overnight fast is no more than about 140 mg/dL. In some embodiments, the individual exhibits normal blood glucose control if the blood glucose level of the individual after the overnight fast is no more than about 139 mg/dL. In some embodiments, the individual exhibits normal blood glucose control if the blood glucose level of the individual after the overnight fast is no more than about 138 mg/dL. In some embodiments, the individual exhibits normal blood glucose control if the blood glucose level of the individual after the overnight fast is no more than about 137 mg/dL. In some embodiments, the individual exhibits normal blood glucose control if the blood glucose level of the individual after the overnight fast is no more than about 136 mg/dL. In some embodiments, the individual exhibits normal blood glucose control if the blood glucose level of the individual after the overnight fast is no more than about 135 mg/dL. In some embodiments, the individual exhibits normal blood glucose control if the blood glucose level of the individual after the overnight fast is no more than about 134 mg/dL. In some embodiments, the individual exhibits normal blood glucose control if the blood glucose level of the individual after the overnight fast is no more than about 133 mg/dL. In some embodiments, the individual exhibits normal blood glucose control if the blood glucose level of the individual after the overnight fast is no more than about 132 mg/dL. In some embodiments, the individual exhibits normal blood glucose control if the blood glucose level of the individual after the overnight fast is no more than about 131 mg/dL. In some embodiments, the individual exhibits normal blood glucose control if the blood glucose level of the individual after the overnight fast is no more than about 130 mg/dL. In some embodiments, the individual exhibits normal blood glucose control if the blood glucose level of the individual after the overnight fast is no more than about 129 mg/dL. In some embodiments, the individual exhibits normal blood glucose control if the blood glucose level of the individual after the overnight fast is no more than about 128 mg/dL. In some embodiments, the individual exhibits normal blood glucose control if the blood glucose level of the individual after the overnight fast is no more than about 127 mg/dL. In some embodiments, the individual exhibits normal blood glucose control if the blood glucose level of the individual after the overnight fast is no more than about 126 mg/dL. In some embodiments, the individual exhibits normal blood glucose control if the blood glucose level of the individual after the overnight fast is no more than about 125 mg/dL. In some embodiments, the individual exhibits normal blood glucose control if the blood glucose level of the individual after the overnight fast is no more than about 124 mg/dL. In some embodiments, the individual exhibits normal blood glucose control if the blood glucose level of the individual after the overnight fast is no more than about 123 mg/dL. In some embodiments, the individual exhibits normal blood glucose control if the blood glucose level of the individual after the overnight fast is no more than about 122 mg/dL. In some embodiments, the individual exhibits normal blood glucose control if the blood glucose level of the individual after the overnight fast is no more than about 121 mg/dL. In some embodiments, the individual exhibits normal blood glucose control if the blood glucose level of the individual after the overnight fast is no more than about 120 mg/dL. In some embodiments, the individual exhibits normal blood glucose control if the blood glucose level of the individual after the overnight fast is no more than about 119 mg/dL. In some embodiments, the individual exhibits normal blood glucose control if the blood glucose level of the individual after the overnight fast is no more than about 118 mg/dL. In some embodiments, the individual exhibits normal blood glucose control if the blood glucose level of the individual after the overnight fast is no more than about 117 mg/dL. In some embodiments, the individual exhibits normal blood glucose control if the blood glucose level of the individual after the overnight fast is no more than about 116 mg/dL. In some embodiments, the individual exhibits normal blood glucose control if the blood glucose level of the individual after the overnight fast is no more than about 115 mg/dL. In some embodiments, the individual exhibits normal blood glucose control if the blood glucose level of the individual after the overnight fast is no more than about 114 mg/dL. In some embodiments, the individual exhibits normal blood glucose control if the blood glucose level of the individual after the overnight fast is no more than about 113 mg/dL. In some embodiments, the individual exhibits normal blood glucose control if the blood glucose level of the individual after the overnight fast is no more than about 112 mg/dL. In some embodiments, the individual exhibits normal blood glucose control if the blood glucose level of the individual after the overnight fast is no more than about 111 mg/dL. In some embodiments, the individual exhibits normal blood glucose control if the blood glucose level of the individual after the overnight fast is no more than about 110 mg/dL. In some embodiments, the individual exhibits normal blood glucose control if the blood glucose level of the individual after the overnight fast is no more than about 109 mg/dL. In some embodiments, the individual exhibits normal blood glucose control if the blood glucose level of the individual after the overnight fast is no more than about 108 mg/dL. In some embodiments, the individual exhibits normal blood glucose control if the blood glucose level of the individual after the overnight fast is no more than about 107 mg/dL. In some embodiments, the individual exhibits normal blood glucose control if the blood glucose level of the individual after the overnight fast is no more than about 106 mg/dL. In some embodiments, the individual exhibits normal blood glucose control if the blood glucose level of the individual after the overnight fast is no more than about 105 mg/dL. In some embodiments, the individual exhibits normal blood glucose control if the blood glucose level of the individual after the overnight fast is no more than about 104 mg/dL. In some embodiments, the individual exhibits normal blood glucose control if the blood glucose level of the individual after the overnight fast is no more than about 103 mg/dL. In some embodiments, the individual exhibits normal blood glucose control if the blood glucose level of the individual after the overnight fast is no more than about 102 mg/dL. In some embodiments, the individual exhibits normal blood glucose control if the blood glucose level of the individual after the overnight fast is no more than about 101 mg/dL. In some embodiments, the individual exhibits normal blood glucose control if the blood glucose level of the individual after the overnight fast is no more than about 100 mg/dL. In some embodiments, the individual exhibits normal blood glucose control if the blood glucose level of the individual after the overnight fast is no more than about 99 mg/dL. In some embodiments, the individual exhibits normal blood glucose control if the blood glucose level of the individual after the overnight fast is no more than about 98 mg/dL. In some embodiments, the individual exhibits normal blood glucose control if the blood glucose level of the individual after the overnight fast is no more than about 97 mg/dL. In some embodiments, the individual exhibits normal blood glucose control if the blood glucose level of the individual after the overnight fast is no more than about 96 mg/dL. In some embodiments, the individual exhibits normal blood glucose control if the blood glucose level of the individual after the overnight fast is no more than about 95 mg/dL. In some embodiments, the individual exhibits normal blood glucose control if the blood glucose level of the individual after the overnight fast is no more than about 94 mg/dL. In some embodiments, the individual exhibits normal blood glucose control if the blood glucose level of the individual after the overnight fast is no more than about 93 mg/dL. In some embodiments, the individual exhibits normal blood glucose control if the blood glucose level of the individual after the overnight fast is no more than about 92 mg/dL. In some embodiments, the individual exhibits normal blood glucose control if the blood glucose level of the individual after the overnight fast is no more than about 91 mg/dL. In some embodiments, the individual exhibits normal blood glucose control if the blood glucose level of the individual after the overnight fast is no more than about 90 mg/dL. In some embodiments, the individual exhibits normal blood glucose control if the blood glucose level of the individual after the overnight fast is no more than about 89 mg/dL. In some embodiments, the individual exhibits normal blood glucose control if the blood glucose level of the individual after the overnight fast is no more than about 88 mg/dL. In some embodiments, the individual exhibits normal blood glucose control if the blood glucose level of the individual after the overnight fast is no more than about 87 mg/dL. In some embodiments, the individual exhibits normal blood glucose control if the blood glucose level of the individual after the overnight fast is no more than about 86 mg/dL. In some embodiments, the individual exhibits normal blood glucose control if the blood glucose level of the individual after the overnight fast is no more than about 85 mg/dL. In some embodiments, the individual exhibits normal blood glucose control if the blood glucose level of the individual after the overnight fast is no more than about 84 mg/dL. In some embodiments, the individual exhibits normal blood glucose control if the blood glucose level of the individual after the overnight fast is no more than about 83 mg/dL. In some embodiments, the individual exhibits normal blood glucose control if the blood glucose level of the individual after the overnight fast is no more than about 82 mg/dL. In some embodiments, the individual exhibits normal blood glucose control if the blood glucose level of the individual after the overnight fast is no more than about 81 mg/dL. In some embodiments, the individual exhibits normal blood glucose control if the blood glucose level of the individual after the overnight fast is no more than about 80 mg/dL. In some embodiments, the individual exhibits normal blood glucose control if the blood glucose level of the individual after the overnight fast is no more than about 79 mg/dL. In some embodiments, the individual exhibits normal blood glucose control if the blood glucose level of the individual after the overnight fast is no more than about 78 mg/dL. In some embodiments, the individual exhibits normal blood glucose control if the blood glucose level of the individual after the overnight fast is no more than about 77 mg/dL. In some embodiments, the individual exhibits normal blood glucose control if the blood glucose level of the individual after the overnight fast is no more than about 76 mg/dL. In some embodiments, the individual exhibits normal blood glucose control if the blood glucose level of the individual after the overnight fast is no more than about 75 mg/dL.

In some embodiments, the individual exhibits normal blood glucose control if the blood glucose level of the individual after the overnight fast is between about 0 and about 150 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or about 150 mg/dL, or any concentration therebetween.

In some embodiments, when the fast lasts about 8 to about 16 hours, the individual exhibits normal blood glucose control if the blood glucose level of the individual after the fast is no more than about 150 mg/dL. In some embodiments, when the fast lasts about 8 to about 16 hours, the individual exhibits normal blood glucose control if the blood glucose level of the individual after the fast is no more than about 149 mg/dL. In some embodiments, when the fast lasts about 8 to about 16 hours, the individual exhibits normal blood glucose control if the blood glucose level of the individual after the fast is no more than about 148 mg/dL. In some embodiments, when the fast lasts about 8 to about 16 hours, the individual exhibits normal blood glucose control if the blood glucose level of the individual after the fast is no more than about 147 mg/dL. In some embodiments, when the fast lasts about 8 to about 16 hours, the individual exhibits normal blood glucose control if the blood glucose level of the individual after the fast is no more than about 146 mg/dL. In some embodiments, when the fast lasts about 8 to about 16 hours, the individual exhibits normal blood glucose control if the blood glucose level of the individual after the fast is no more than about 145 mg/dL. In some embodiments, when the fast lasts about 8 to about 16 hours, the individual exhibits normal blood glucose control if the blood glucose level of the individual after the fast is no more than about 144 mg/dL. In some embodiments, when the fast lasts about 8 to about 16 hours, the individual exhibits normal blood glucose control if the blood glucose level of the individual after the fast is no more than about 143 mg/dL. In some embodiments, when the fast lasts about 8 to about 16 hours, the individual exhibits normal blood glucose control if the blood glucose level of the individual after the fast is no more than about 142 mg/dL. In some embodiments, when the fast lasts about 8 to about 16 hours, the individual exhibits normal blood glucose control if the blood glucose level of the individual after the fast is no more than about 141 mg/dL. In some embodiments, when the fast lasts about 8 to about 16 hours, the individual exhibits normal blood glucose control if the blood glucose level of the individual after the fast is no more than about 140 mg/dL. In some embodiments, when the fast lasts about 8 to about 16 hours, the individual exhibits normal blood glucose control if the blood glucose level of the individual after the fast is no more than about 139 mg/dL. In some embodiments, when the fast lasts about 8 to about 16 hours, the individual exhibits normal blood glucose control if the blood glucose level of the individual after the fast is no more than about 138 mg/dL. In some embodiments, when the fast lasts about 8 to about 16 hours, the individual exhibits normal blood glucose control if the blood glucose level of the individual after the fast is no more than about 137 mg/dL. In some embodiments, when the fast lasts about 8 to about 16 hours, the individual exhibits normal blood glucose control if the blood glucose level of the individual after the fast is no more than about 136 mg/dL. In some embodiments, when the fast lasts about 8 to about 16 hours, the individual exhibits normal blood glucose control if the blood glucose level of the individual after the fast is no more than about 135 mg/dL. In some embodiments, when the fast lasts about 8 to about 16 hours, the individual exhibits normal blood glucose control if the blood glucose level of the individual after the fast is no more than about 134 mg/dL. In some embodiments, when the fast lasts about 8 to about 16 hours, the individual exhibits normal blood glucose control if the blood glucose level of the individual after the fast is no more than about 133 mg/dL. In some embodiments, when the fast lasts about 8 to about 16 hours, the individual exhibits normal blood glucose control if the blood glucose level of the individual after the fast is no more than about 132 mg/dL. In some embodiments, when the fast lasts about 8 to about 16 hours, the individual exhibits normal blood glucose control if the blood glucose level of the individual after the fast is no more than about 131 mg/dL. In some embodiments, when the fast lasts about 8 to about 16 hours, the individual exhibits normal blood glucose control if the blood glucose level of the individual after the fast is no more than about 130 mg/dL. In some embodiments, when the fast lasts about 8 to about 16 hours, the individual exhibits normal blood glucose control if the blood glucose level of the individual after the fast is no more than about 129 mg/dL. In some embodiments, when the fast lasts about 8 to about 16 hours, the individual exhibits normal blood glucose control if the blood glucose level of the individual after the fast is no more than about 128 mg/dL. In some embodiments, when the fast lasts about 8 to about 16 hours, the individual exhibits normal blood glucose control if the blood glucose level of the individual after the fast is no more than about 127 mg/dL. In some embodiments, when the fast lasts about 8 to about 16 hours, the individual exhibits normal blood glucose control if the blood glucose level of the individual after the fast is no more than about 126 mg/dL. In some embodiments, when the fast lasts about 8 to about 16 hours, the individual exhibits normal blood glucose control if the blood glucose level of the individual after the fast is no more than about 125 mg/dL. In some embodiments, when the fast lasts about 8 to about 16 hours, the individual exhibits normal blood glucose control if the blood glucose level of the individual after the fast is no more than about 124 mg/dL. In some embodiments, when the fast lasts about 8 to about 16 hours, the individual exhibits normal blood glucose control if the blood glucose level of the individual after the fast is no more than about 123 mg/dL. In some embodiments, when the fast lasts about 8 to about 16 hours, the individual exhibits normal blood glucose control if the blood glucose level of the individual after the fast is no more than about 122 mg/dL. In some embodiments, when the fast lasts about 8 to about 16 hours, the individual exhibits normal blood glucose control if the blood glucose level of the individual after the fast is no more than about 121 mg/dL. In some embodiments, when the fast lasts about 8 to about 16 hours, the individual exhibits normal blood glucose control if the blood glucose level of the individual after the fast is no more than about 120 mg/dL. In some embodiments, when the fast lasts about 8 to about 16 hours, the individual exhibits normal blood glucose control if the blood glucose level of the individual after the fast is no more than about 119 mg/dL. In some embodiments, when the fast lasts about 8 to about 16 hours, the individual exhibits normal blood glucose control if the blood glucose level of the individual after the fast is no more than about 118 mg/dL. In some embodiments, when the fast lasts about 8 to about 16 hours, the individual exhibits normal blood glucose control if the blood glucose level of the individual after the fast is no more than about 117 mg/dL. In some embodiments, when the fast lasts about 8 to about 16 hours, the individual exhibits normal blood glucose control if the blood glucose level of the individual after the fast is no more than about 116 mg/dL. In some embodiments, when the fast lasts about 8 to about 16 hours, the individual exhibits normal blood glucose control if the blood glucose level of the individual after the fast is no more than about 115 mg/dL. In some embodiments, when the fast lasts about 8 to about 16 hours, the individual exhibits normal blood glucose control if the blood glucose level of the individual after the fast is no more than about 114 mg/dL. In some embodiments, when the fast lasts about 8 to about 16 hours, the individual exhibits normal blood glucose control if the blood glucose level of the individual after the fast is no more than about 113 mg/dL. In some embodiments, when the fast lasts about 8 to about 16 hours, the individual exhibits normal blood glucose control if the blood glucose level of the individual after the fast is no more than about 112 mg/dL. In some embodiments, when the fast lasts about 8 to about 16 hours, the individual exhibits normal blood glucose control if the blood glucose level of the individual after the fast is no more than about 111 mg/dL. In some embodiments, when the fast lasts about 8 to about 16 hours, the individual exhibits normal blood glucose control if the blood glucose level of the individual after the fast is no more than about 110 mg/dL. In some embodiments, when the fast lasts about 8 to about 16 hours, the individual exhibits normal blood glucose control if the blood glucose level of the individual after the fast is no more than about 109 mg/dL. In some embodiments, when the fast lasts about 8 to about 16 hours, the individual exhibits normal blood glucose control if the blood glucose level of the individual after the fast is no more than about 108 mg/dL. In some embodiments, when the fast lasts about 8 to about 16 hours, the individual exhibits normal blood glucose control if the blood glucose level of the individual after the fast is no more than about 107 mg/dL. In some embodiments, when the fast lasts about 8 to about 16 hours, the individual exhibits normal blood glucose control if the blood glucose level of the individual after the fast is no more than about 106 mg/dL. In some embodiments, when the fast lasts about 8 to about 16 hours, the individual exhibits normal blood glucose control if the blood glucose level of the individual after the fast is no more than about 105 mg/dL. In some embodiments, when the fast lasts about 8 to about 16 hours, the individual exhibits normal blood glucose control if the blood glucose level of the individual after the fast is no more than about 104 mg/dL. In some embodiments, when the fast lasts about 8 to about 16 hours, the individual exhibits normal blood glucose control if the blood glucose level of the individual after the fast is no more than about 103 mg/dL. In some embodiments, when the fast lasts about 8 to about 16 hours, the individual exhibits normal blood glucose control if the blood glucose level of the individual after the fast is no more than about 102 mg/dL. In some embodiments, when the fast lasts about 8 to about 16 hours, the individual exhibits normal blood glucose control if the blood glucose level of the individual after the fast is no more than about 101 mg/dL. In some embodiments, when the fast lasts about 8 to about 16 hours, the individual exhibits normal blood glucose control if the blood glucose level of the individual after the fast is no more than about 100 mg/dL. In some embodiments, when the fast lasts about 8 to about 16 hours, the individual exhibits normal blood glucose control if the blood glucose level of the individual after the fast is no more than about 99 mg/dL. In some embodiments, when the fast lasts about 8 to about 16 hours, the individual exhibits normal blood glucose control if the blood glucose level of the individual after the fast is no more than about 98 mg/dL. In some embodiments, when the fast lasts about 8 to about 16 hours, the individual exhibits normal blood glucose control if the blood glucose level of the individual after the fast is no more than about 97 mg/dL. In some embodiments, when the fast lasts about 8 to about 16 hours, the individual exhibits normal blood glucose control if the blood glucose level of the individual after the fast is no more than about 96 mg/dL. In some embodiments, when the fast lasts about 8 to about 16 hours, the individual exhibits normal blood glucose control if the blood glucose level of the individual after the fast is no more than about 95 mg/dL. In some embodiments, when the fast lasts about 8 to about 16 hours, the individual exhibits normal blood glucose control if the blood glucose level of the individual after the fast is no more than about 94 mg/dL. In some embodiments, when the fast lasts about 8 to about 16 hours, the individual exhibits normal blood glucose control if the blood glucose level of the individual after the fast is no more than about 93 mg/dL. In some embodiments, when the fast lasts about 8 to about 16 hours, the individual exhibits normal blood glucose control if the blood glucose level of the individual after the fast is no more than about 92 mg/dL. In some embodiments, when the fast lasts about 8 to about 16 hours, the individual exhibits normal blood glucose control if the blood glucose level of the individual after the fast is no more than about 91 mg/dL. In some embodiments, when the fast lasts about 8 to about 16 hours, the individual exhibits normal blood glucose control if the blood glucose level of the individual after the fast is no more than about 90 mg/dL. In some embodiments, when the fast lasts about 8 to about 16 hours, the individual exhibits normal blood glucose control if the blood glucose level of the individual after the fast is no more than about 89 mg/dL. In some embodiments, when the fast lasts about 8 to about 16 hours, the individual exhibits normal blood glucose control if the blood glucose level of the individual after the fast is no more than about 88 mg/dL. In some embodiments, when the fast lasts about 8 to about 16 hours, the individual exhibits normal blood glucose control if the blood glucose level of the individual after the fast is no more than about 87 mg/dL. In some embodiments, when the fast lasts about 8 to about 16 hours, the individual exhibits normal blood glucose control if the blood glucose level of the individual after the fast is no more than about 86 mg/dL. In some embodiments, when the fast lasts about 8 to about 16 hours, the individual exhibits normal blood glucose control if the blood glucose level of the individual after the fast is no more than about 85 mg/dL. In some embodiments, when the fast lasts about 8 to about 16 hours, the individual exhibits normal blood glucose control if the blood glucose level of the individual after the fast is no more than about 84 mg/dL. In some embodiments, when the fast lasts about 8 to about 16 hours, the individual exhibits normal blood glucose control if the blood glucose level of the individual after the fast is no more than about 83 mg/dL. In some embodiments, when the fast lasts about 8 to about 16 hours, the individual exhibits normal blood glucose control if the blood glucose level of the individual after the fast is no more than about 82 mg/dL. In some embodiments, when the fast lasts about 8 to about 16 hours, the individual exhibits normal blood glucose control if the blood glucose level of the individual after the fast is no more than about 81 mg/dL. In some embodiments, when the fast lasts about 8 to about 16 hours, the individual exhibits normal blood glucose control if the blood glucose level of the individual after the fast is no more than about 80 mg/dL. In some embodiments, when the fast lasts about 8 to about 16 hours, the individual exhibits normal blood glucose control if the blood glucose level of the individual after the fast is no more than about 79 mg/dL. In some embodiments, when the fast lasts about 8 to about 16 hours, the individual exhibits normal blood glucose control if the blood glucose level of the individual after the fast is no more than about 78 mg/dL. In some embodiments, when the fast lasts about 8 to about 16 hours, the individual exhibits normal blood glucose control if the blood glucose level of the individual after the fast is no more than about 77 mg/dL. In some embodiments, when the fast lasts about 8 to about 16 hours, the individual exhibits normal blood glucose control if the blood glucose level of the individual after the fast is no more than about 76 mg/dL. In some embodiments, when the fast lasts about 8 to about 16 hours, the individual exhibits normal blood glucose control if the blood glucose level of the individual after the fast is no more than about 75 mg/dL.

In some embodiments, normal blood glucose control is established the day of administration of an IGF1R inhibitor. In some embodiments, normal blood glucose control is established 1, 2, 3, 4, 5, 6, 7, 8, 9, 10, 11, 12, 13, 14, 21, 28, 35, 42, 48 or 56 days prior to administration of an IGF1R inhibitor.

In some embodiments, when the fast lasts about 8 hours to about 16 hours, the individual exhibits normal blood glucose control if the blood glucose level of the individual after the fast is between 0 and about 150 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or about 150 mg/dL, or any concentration therebetween.

In some embodiments, the individual exhibits abnormal blood glucose control if the blood glucose level of the individual after the fast is above about 150 mg/dL. In some embodiments, the individual exhibits abnormal blood glucose control if the blood glucose level of the individual after the fast is above about 149 mg/dL. In some embodiments, the individual exhibits abnormal blood glucose control if the blood glucose level of the individual after the fast is above about 148 mg/dL. In some embodiments, the individual exhibits abnormal blood glucose control if the blood glucose level of the individual after the fast is above about 147 mg/dL. In some embodiments, the individual exhibits abnormal blood glucose control if the blood glucose level of the individual after the fast is above about 146 mg/dL. In some embodiments, the individual exhibits abnormal blood glucose control if the blood glucose level of the individual after the fast is above about 145 mg/dL. In some embodiments, the individual exhibits abnormal blood glucose control if the blood glucose level of the individual after the fast is above about 144 mg/dL. In some embodiments, the individual exhibits abnormal blood glucose control if the blood glucose level of the individual after the fast is above about 143 mg/dL. In some embodiments, the individual exhibits abnormal blood glucose control if the blood glucose level of the individual after the fast is above about 142 mg/dL. In some embodiments, the individual exhibits abnormal blood glucose control if the blood glucose level of the individual after the fast is above about 141 mg/dL. In some embodiments, the individual exhibits abnormal blood glucose control if the blood glucose level of the individual after the fast is above about 140 mg/dL. In some embodiments, the individual exhibits abnormal blood glucose control if the blood glucose level of the individual after the fast is above about 139 mg/dL. In some embodiments, the individual exhibits abnormal blood glucose control if the blood glucose level of the individual after the fast is above about 138 mg/dL. In some embodiments, the individual exhibits abnormal blood glucose control if the blood glucose level of the individual after the fast is above about 137 mg/dL. In some embodiments, the individual exhibits abnormal blood glucose control if the blood glucose level of the individual after the fast is above about 136 mg/dL. In some embodiments, the individual exhibits abnormal blood glucose control if the blood glucose level of the individual after the fast is above about 135 mg/dL. In some embodiments, the individual exhibits abnormal blood glucose control if the blood glucose level of the individual after the fast is above about 134 mg/dL. In some embodiments, the individual exhibits abnormal blood glucose control if the blood glucose level of the individual after the fast is above about 133 mg/dL. In some embodiments, the individual exhibits abnormal blood glucose control if the blood glucose level of the individual after the fast is above about 132 mg/dL. In some embodiments, the individual exhibits abnormal blood glucose control if the blood glucose level of the individual after the fast is above about 131 mg/dL. In some embodiments, the individual exhibits abnormal blood glucose control if the blood glucose level of the individual after the fast is above about 130 mg/dL. In some embodiments, the individual exhibits abnormal blood glucose control if the blood glucose level of the individual after the fast is above about 129 mg/dL. In some embodiments, the individual exhibits abnormal blood glucose control if the blood glucose level of the individual after the fast is above about 128 mg/dL. In some embodiments, the individual exhibits abnormal blood glucose control if the blood glucose level of the individual after the fast is above about 127 mg/dL. In some embodiments, the individual exhibits abnormal blood glucose control if the blood glucose level of the individual after the fast is above about 126 mg/dL. In some embodiments, the individual exhibits abnormal blood glucose control if the blood glucose level of the individual after the fast is above about 125 mg/dL. In some embodiments, the individual exhibits abnormal blood glucose control if the blood glucose level of the individual after the fast is above about 124 mg/dL. In some embodiments, the individual exhibits abnormal blood glucose control if the blood glucose level of the individual after the fast is above about 123 mg/dL. In some embodiments, the individual exhibits abnormal blood glucose control if the blood glucose level of the individual after the fast is above about 122 mg/dL. In some embodiments, the individual exhibits abnormal blood glucose control if the blood glucose level of the individual after the fast is above about 121 mg/dL. In some embodiments, the individual exhibits abnormal blood glucose control if the blood glucose level of the individual after the fast is above about 120 mg/dL. In some embodiments, the individual exhibits abnormal blood glucose control if the blood glucose level of the individual after the fast is above about 119 mg/dL. In some embodiments, the individual exhibits abnormal blood glucose control if the blood glucose level of the individual after the fast is above about 118 mg/dL. In some embodiments, the individual exhibits abnormal blood glucose control if the blood glucose level of the individual after the fast is above about 117 mg/dL. In some embodiments, the individual exhibits abnormal blood glucose control if the blood glucose level of the individual after the fast is above about 116 mg/dL. In some embodiments, the individual exhibits abnormal blood glucose control if the blood glucose level of the individual after the fast is above about 115 mg/dL. In some embodiments, the individual exhibits abnormal blood glucose control if the blood glucose level of the individual after the fast is above about 114 mg/dL. In some embodiments, the individual exhibits abnormal blood glucose control if the blood glucose level of the individual after the fast is above about 113 mg/dL. In some embodiments, the individual exhibits abnormal blood glucose control if the blood glucose level of the individual after the fast is above about 112 mg/dL. In some embodiments, the individual exhibits abnormal blood glucose control if the blood glucose level of the individual after the fast is above about 111 mg/dL. In some embodiments, the individual exhibits abnormal blood glucose control if the blood glucose level of the individual after the fast is above about 110 mg/dL. In some embodiments, the individual exhibits abnormal blood glucose control if the blood glucose level of the individual after the fast is above about 109 mg/dL. In some embodiments, the individual exhibits abnormal blood glucose control if the blood glucose level of the individual after the fast is above about 108 mg/dL. In some embodiments, the individual exhibits abnormal blood glucose control if the blood glucose level of the individual after the fast is above about 107 mg/dL. In some embodiments, the individual exhibits abnormal blood glucose control if the blood glucose level of the individual after the fast is above about 106 mg/dL. In some embodiments, the individual exhibits abnormal blood glucose control if the blood glucose level of the individual after the fast is above about 105 mg/dL. In some embodiments, the individual exhibits abnormal blood glucose control if the blood glucose level of the individual after the fast is above about 104 mg/dL. In some embodiments, the individual exhibits abnormal blood glucose control if the blood glucose level of the individual after the fast is above about 103 mg/dL. In some embodiments, the individual exhibits abnormal blood glucose control if the blood glucose level of the individual after the fast is above about 102 mg/dL. In some embodiments, the individual exhibits abnormal blood glucose control if the blood glucose level of the individual after the fast is above about 101 mg/dL. In some embodiments, the individual exhibits abnormal blood glucose control if the blood glucose level of the individual after the fast is above about 100 mg/dL. In some embodiments, the individual exhibits abnormal blood glucose control if the blood glucose level of the individual after the fast is above about 99 mg/dL. In some embodiments, the individual exhibits abnormal blood glucose control if the blood glucose level of the individual after the fast is above about 98 mg/dL. In some embodiments, the individual exhibits abnormal blood glucose control if the blood glucose level of the individual after the fast is above about 97 mg/dL. In some embodiments, the individual exhibits abnormal blood glucose control if the blood glucose level of the individual after the fast is above about 96 mg/dL. In some embodiments, the individual exhibits abnormal blood glucose control if the blood glucose level of the individual after the fast is above about 95 mg/dL. In some embodiments, the individual exhibits abnormal blood glucose control if the blood glucose level of the individual after the fast is above about 94 mg/dL. In some embodiments, the individual exhibits abnormal blood glucose control if the blood glucose level of the individual after the fast is above about 93 mg/dL. In some embodiments, the individual exhibits abnormal blood glucose control if the blood glucose level of the individual after the fast is above about 92 mg/dL. In some embodiments, the individual exhibits abnormal blood glucose control if the blood glucose level of the individual after the fast is above about 91 mg/dL. In some embodiments, the individual exhibits abnormal blood glucose control if the blood glucose level of the individual after the fast is above about 90 mg/dL. In some embodiments, the individual exhibits abnormal blood glucose control if the blood glucose level of the individual after the fast is above about 89 mg/dL. In some embodiments, the individual exhibits abnormal blood glucose control if the blood glucose level of the individual after the fast is above about 88 mg/dL. In some embodiments, the individual exhibits abnormal blood glucose control if the blood glucose level of the individual after the fast is above about 87 mg/dL. In some embodiments, the individual exhibits abnormal blood glucose control if the blood glucose level of the individual after the fast is above about 86 mg/dL. In some embodiments, the individual exhibits abnormal blood glucose control if the blood glucose level of the individual after the fast is above about 85 mg/dL. In some embodiments, the individual exhibits abnormal blood glucose control if the blood glucose level of the individual after the fast is above about 84 mg/dL. In some embodiments, the individual exhibits abnormal blood glucose control if the blood glucose level of the individual after the fast is above about 83 mg/dL. In some embodiments, the individual exhibits abnormal blood glucose control if the blood glucose level of the individual after the fast is above about 82 mg/dL. In some embodiments, the individual exhibits abnormal blood glucose control if the blood glucose level of the individual after the fast is above about 81 mg/dL. In some embodiments, the individual exhibits abnormal blood glucose control if the blood glucose level of the individual after the fast is above about 80 mg/dL. In some embodiments, the individual exhibits abnormal blood glucose control if the blood glucose level of the individual after the fast is above about 79 mg/dL. In some embodiments, the individual exhibits abnormal blood glucose control if the blood glucose level of the individual after the fast is above about 78 mg/dL. In some embodiments, the individual exhibits abnormal blood glucose control if the blood glucose level of the individual after the fast is above about 77 mg/dL. In some embodiments, the individual exhibits abnormal blood glucose control if the blood glucose level of the individual after the fast is above about 76 mg/dL. In some embodiments, the individual exhibits abnormal blood glucose control if the blood glucose level of the individual after the fast is above about 75 mg/dL.

In some embodiments, the individual exhibits abnormal blood glucose control if the blood glucose level of the individual after the fast is above about 75 mg/dL, e.g., about 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or 150 mg/dL, or any concentration above 150 mg/dL.

In some embodiments, the individual exhibits abnormal blood glucose control if the blood glucose level of the individual after the overnight fast is above about 150 mg/dL. In some embodiments, the individual exhibits abnormal blood glucose control if the blood glucose level of the individual after the overnight fast is above about 149 mg/dL. In some embodiments, the individual exhibits abnormal blood glucose control if the blood glucose level of the individual after the overnight fast is above about 148 mg/dL. In some embodiments, the individual exhibits abnormal blood glucose control if the blood glucose level of the individual after the overnight fast is above about 147 mg/dL. In some embodiments, the individual exhibits abnormal blood glucose control if the blood glucose level of the individual after the overnight fast is above about 146 mg/dL. In some embodiments, the individual exhibits abnormal blood glucose control if the blood glucose level of the individual after the overnight fast is above about 145 mg/dL. In some embodiments, the individual exhibits abnormal blood glucose control if the blood glucose level of the individual after the overnight fast is above about 144 mg/dL. In some embodiments, the individual exhibits abnormal blood glucose control if the blood glucose level of the individual after the overnight fast is above about 143 mg/dL. In some embodiments, the individual exhibits abnormal blood glucose control if the blood glucose level of the individual after the overnight fast is above about 142 mg/dL. In some embodiments, the individual exhibits abnormal blood glucose control if the blood glucose level of the individual after the overnight fast is above about 141 mg/dL. In some embodiments, the individual exhibits abnormal blood glucose control if the blood glucose level of the individual after the overnight fast is above about 140 mg/dL. In some embodiments, the individual exhibits abnormal blood glucose control if the blood glucose level of the individual after the overnight fast is above about 139 mg/dL. In some embodiments, the individual exhibits abnormal blood glucose control if the blood glucose level of the individual after the overnight fast is above about 138 mg/dL. In some embodiments, the individual exhibits abnormal blood glucose control if the blood glucose level of the individual after the overnight fast is above about 137 mg/dL. In some embodiments, the individual exhibits abnormal blood glucose control if the blood glucose level of the individual after the overnight fast is above about 136 mg/dL. In some embodiments, the individual exhibits abnormal blood glucose control if the blood glucose level of the individual after the overnight fast is above about 135 mg/dL. In some embodiments, the individual exhibits abnormal blood glucose control if the blood glucose level of the individual after the overnight fast is above about 134 mg/dL. In some embodiments, the individual exhibits abnormal blood glucose control if the blood glucose level of the individual after the overnight fast is above about 133 mg/dL. In some embodiments, the individual exhibits abnormal blood glucose control if the blood glucose level of the individual after the overnight fast is above about 132 mg/dL. In some embodiments, the individual exhibits abnormal blood glucose control if the blood glucose level of the individual after the overnight fast is above about 131 mg/dL. In some embodiments, the individual exhibits abnormal blood glucose control if the blood glucose level of the individual after the overnight fast is above about 130 mg/dL. In some embodiments, the individual exhibits abnormal blood glucose control if the blood glucose level of the individual after the overnight fast is above about 129 mg/dL. In some embodiments, the individual exhibits abnormal blood glucose control if the blood glucose level of the individual after the overnight fast is above about 128 mg/dL. In some embodiments, the individual exhibits abnormal blood glucose control if the blood glucose level of the individual after the overnight fast is above about 127 mg/dL. In some embodiments, the individual exhibits abnormal blood glucose control if the blood glucose level of the individual after the overnight fast is above about 126 mg/dL. In some embodiments, the individual exhibits abnormal blood glucose control if the blood glucose level of the individual after the overnight fast is above about 125 mg/dL. In some embodiments, the individual exhibits abnormal blood glucose control if the blood glucose level of the individual after the overnight fast is above about 124 mg/dL. In some embodiments, the individual exhibits abnormal blood glucose control if the blood glucose level of the individual after the overnight fast is above about 123 mg/dL. In some embodiments, the individual exhibits abnormal blood glucose control if the blood glucose level of the individual after the overnight fast is above about 122 mg/dL. In some embodiments, the individual exhibits abnormal blood glucose control if the blood glucose level of the individual after the overnight fast is above about 121 mg/dL. In some embodiments, the individual exhibits abnormal blood glucose control if the blood glucose level of the individual after the overnight fast is above about 120 mg/dL. In some embodiments, the individual exhibits abnormal blood glucose control if the blood glucose level of the individual after the overnight fast is above about 119 mg/dL. In some embodiments, the individual exhibits abnormal blood glucose control if the blood glucose level of the individual after the overnight fast is above about 118 mg/dL. In some embodiments, the individual exhibits abnormal blood glucose control if the blood glucose level of the individual after the overnight fast is above about 117 mg/dL. In some embodiments, the individual exhibits abnormal blood glucose control if the blood glucose level of the individual after the overnight fast is above about 116 mg/dL. In some embodiments, the individual exhibits abnormal blood glucose control if the blood glucose level of the individual after the overnight fast is above about 115 mg/dL. In some embodiments, the individual exhibits abnormal blood glucose control if the blood glucose level of the individual after the overnight fast is above about 114 mg/dL. In some embodiments, the individual exhibits abnormal blood glucose control if the blood glucose level of the individual after the overnight fast is above about 113 mg/dL. In some embodiments, the individual exhibits abnormal blood glucose control if the blood glucose level of the individual after the overnight fast is above about 112 mg/dL. In some embodiments, the individual exhibits abnormal blood glucose control if the blood glucose level of the individual after the overnight fast is above about 111 mg/dL. In some embodiments, the individual exhibits abnormal blood glucose control if the blood glucose level of the individual after the overnight fast is above about 110 mg/dL. In some embodiments, the individual exhibits abnormal blood glucose control if the blood glucose level of the individual after the overnight fast is above about 109 mg/dL. In some embodiments, the individual exhibits abnormal blood glucose control if the blood glucose level of the individual after the overnight fast is above about 108 mg/dL. In some embodiments, the individual exhibits abnormal blood glucose control if the blood glucose level of the individual after the overnight fast is above about 107 mg/dL. In some embodiments, the individual exhibits abnormal blood glucose control if the blood glucose level of the individual after the overnight fast is above about 106 mg/dL. In some embodiments, the individual exhibits abnormal blood glucose control if the blood glucose level of the individual after the overnight fast is above about 105 mg/dL. In some embodiments, the individual exhibits abnormal blood glucose control if the blood glucose level of the individual after the overnight fast is above about 104 mg/dL. In some embodiments, the individual exhibits abnormal blood glucose control if the blood glucose level of the individual after the overnight fast is above about 103 mg/dL. In some embodiments, the individual exhibits abnormal blood glucose control if the blood glucose level of the individual after the overnight fast is above about 102 mg/dL. In some embodiments, the individual exhibits abnormal blood glucose control if the blood glucose level of the individual after the overnight fast is above about 101 mg/dL. In some embodiments, the individual exhibits abnormal blood glucose control if the blood glucose level of the individual after the overnight fast is above about 100 mg/dL. In some embodiments, the individual exhibits abnormal blood glucose control if the blood glucose level of the individual after the overnight fast is above about 99 mg/dL. In some embodiments, the individual exhibits abnormal blood glucose control if the blood glucose level of the individual after the overnight fast is above about 98 mg/dL. In some embodiments, the individual exhibits abnormal blood glucose control if the blood glucose level of the individual after the overnight fast is above about 97 mg/dL. In some embodiments, the individual exhibits abnormal blood glucose control if the blood glucose level of the individual after the overnight fast is above about 96 mg/dL. In some embodiments, the individual exhibits abnormal blood glucose control if the blood glucose level of the individual after the overnight fast is above about 95 mg/dL. In some embodiments, the individual exhibits abnormal blood glucose control if the blood glucose level of the individual after the overnight fast is above about 94 mg/dL. In some embodiments, the individual exhibits abnormal blood glucose control if the blood glucose level of the individual after the overnight fast is above about 93 mg/dL. In some embodiments, the individual exhibits abnormal blood glucose control if the blood glucose level of the individual after the overnight fast is above about 92 mg/dL. In some embodiments, the individual exhibits abnormal blood glucose control if the blood glucose level of the individual after the overnight fast is above about 91 mg/dL. In some embodiments, the individual exhibits abnormal blood glucose control if the blood glucose level of the individual after the overnight fast is above about 90 mg/dL. In some embodiments, the individual exhibits abnormal blood glucose control if the blood glucose level of the individual after the overnight fast is above about 89 mg/dL. In some embodiments, the individual exhibits abnormal blood glucose control if the blood glucose level of the individual after the overnight fast is above about 88 mg/dL. In some embodiments, the individual exhibits abnormal blood glucose control if the blood glucose level of the individual after the overnight fast is above about 87 mg/dL. In some embodiments, the individual exhibits abnormal blood glucose control if the blood glucose level of the individual after the overnight fast is above about 86 mg/dL. In some embodiments, the individual exhibits abnormal blood glucose control if the blood glucose level of the individual after the overnight fast is above about 85 mg/dL. In some embodiments, the individual exhibits abnormal blood glucose control if the blood glucose level of the individual after the overnight fast is above about 84 mg/dL. In some embodiments, the individual exhibits abnormal blood glucose control if the blood glucose level of the individual after the overnight fast is above about 83 mg/dL. In some embodiments, the individual exhibits abnormal blood glucose control if the blood glucose level of the individual after the overnight fast is above about 82 mg/dL. In some embodiments, the individual exhibits abnormal blood glucose control if the blood glucose level of the individual after the overnight fast is above about 81 mg/dL. In some embodiments, the individual exhibits abnormal blood glucose control if the blood glucose level of the individual after the overnight fast is above about 80 mg/dL. In some embodiments, the individual exhibits abnormal blood glucose control if the blood glucose level of the individual after the overnight fast is above about 79 mg/dL. In some embodiments, the individual exhibits abnormal blood glucose control if the blood glucose level of the individual after the overnight fast is above about 78 mg/dL. In some embodiments, the individual exhibits abnormal blood glucose control if the blood glucose level of the individual after the overnight fast is above about 77 mg/dL. In some embodiments, the individual exhibits abnormal blood glucose control if the blood glucose level of the individual after the overnight fast is above about 76 mg/dL. In some embodiments, the individual exhibits abnormal blood glucose control if the blood glucose level of the individual after the overnight fast is above about 75 mg/dL.

In some embodiments, the individual exhibits abnormal blood glucose control if the blood glucose level of the individual after the overnight fast is above about 75 mg/dL, e.g., about 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or about 150 mg/dL, or any concentration above 150 mg/dL.

In some embodiments, when the fast lasts about 8 to about 16 hours, the individual exhibits abnormal blood glucose control if the blood glucose level of the individual after the fast is above about 150 mg/dL. In some embodiments, when the fast lasts about 8 to about 16 hours, the individual exhibits abnormal blood glucose control if the blood glucose level of the individual after the fast is above about 149 mg/dL. In some embodiments, when the fast lasts about 8 to about 16 hours, the individual exhibits abnormal blood glucose control if the blood glucose level of the individual after the fast is above about 148 mg/dL. In some embodiments, when the fast lasts about 8 to about 16 hours, the individual exhibits abnormal blood glucose control if the blood glucose level of the individual after the fast is above about 147 mg/dL. In some embodiments, when the fast lasts about 8 to about 16 hours, the individual exhibits abnormal blood glucose control if the blood glucose level of the individual after the fast is above about 146 mg/dL. In some embodiments, when the fast lasts about 8 to about 16 hours, the individual exhibits abnormal blood glucose control if the blood glucose level of the individual after the fast is above about 145 mg/dL. In some embodiments, when the fast lasts about 8 to about 16 hours, the individual exhibits abnormal blood glucose control if the blood glucose level of the individual after the fast is above about 144 mg/dL. In some embodiments, when the fast lasts about 8 to about 16 hours, the individual exhibits abnormal blood glucose control if the blood glucose level of the individual after the fast is above about 143 mg/dL. In some embodiments, when the fast lasts about 8 to about 16 hours, the individual exhibits abnormal blood glucose control if the blood glucose level of the individual after the fast is above about 142 mg/dL. In some embodiments, when the fast lasts about 8 to about 16 hours, the individual exhibits abnormal blood glucose control if the blood glucose level of the individual after the fast is above about 141 mg/dL. In some embodiments, when the fast lasts about 8 to about 16 hours, the individual exhibits abnormal blood glucose control if the blood glucose level of the individual after the fast is above about 140 mg/dL. In some embodiments, when the fast lasts about 8 to about 16 hours, the individual exhibits abnormal blood glucose control if the blood glucose level of the individual after the fast is above about 139 mg/dL. In some embodiments, when the fast lasts about 8 to about 16 hours, the individual exhibits abnormal blood glucose control if the blood glucose level of the individual after the fast is above about 138 mg/dL. In some embodiments, when the fast lasts about 8 to about 16 hours, the individual exhibits abnormal blood glucose control if the blood glucose level of the individual after the fast is above about 137 mg/dL. In some embodiments, when the fast lasts about 8 to about 16 hours, the individual exhibits abnormal blood glucose control if the blood glucose level of the individual after the fast is above about 136 mg/dL. In some embodiments, when the fast lasts about 8 to about 16 hours, the individual exhibits abnormal blood glucose control if the blood glucose level of the individual after the fast is above about 135 mg/dL. In some embodiments, when the fast lasts about 8 to about 16 hours, the individual exhibits abnormal blood glucose control if the blood glucose level of the individual after the fast is above about 134 mg/dL. In some embodiments, when the fast lasts about 8 to about 16 hours, the individual exhibits abnormal blood glucose control if the blood glucose level of the individual after the fast is above about 133 mg/dL. In some embodiments, when the fast lasts about 8 to about 16 hours, the individual exhibits abnormal blood glucose control if the blood glucose level of the individual after the fast is above about 132 mg/dL. In some embodiments, when the fast lasts about 8 to about 16 hours, the individual exhibits abnormal blood glucose control if the blood glucose level of the individual after the fast is above about 131 mg/dL. In some embodiments, when the fast lasts about 8 to about 16 hours, the individual exhibits abnormal blood glucose control if the blood glucose level of the individual after the fast is above about 130 mg/dL. In some embodiments, when the fast lasts about 8 to about 16 hours, the individual exhibits abnormal blood glucose control if the blood glucose level of the individual after the fast is above about 129 mg/dL. In some embodiments, when the fast lasts about 8 to about 16 hours, the individual exhibits abnormal blood glucose control if the blood glucose level of the individual after the fast is above about 128 mg/dL. In some embodiments, when the fast lasts about 8 to about 16 hours, the individual exhibits abnormal blood glucose control if the blood glucose level of the individual after the fast is above about 127 mg/dL. In some embodiments, when the fast lasts about 8 to about 16 hours, the individual exhibits abnormal blood glucose control if the blood glucose level of the individual after the fast is above about 126 mg/dL. In some embodiments, when the fast lasts about 8 to about 16 hours, the individual exhibits abnormal blood glucose control if the blood glucose level of the individual after the fast is above about 125 mg/dL. In some embodiments, when the fast lasts about 8 to about 16 hours, the individual exhibits abnormal blood glucose control if the blood glucose level of the individual after the fast is above about 124 mg/dL. In some embodiments, when the fast lasts about 8 to about 16 hours, the individual exhibits abnormal blood glucose control if the blood glucose level of the individual after the fast is above about 123 mg/dL. In some embodiments, when the fast lasts about 8 to about 16 hours, the individual exhibits abnormal blood glucose control if the blood glucose level of the individual after the fast is above about 122 mg/dL. In some embodiments, when the fast lasts about 8 to about 16 hours, the individual exhibits abnormal blood glucose control if the blood glucose level of the individual after the fast is above about 121 mg/dL. In some embodiments, when the fast lasts about 8 to about 16 hours, the individual exhibits abnormal blood glucose control if the blood glucose level of the individual after the fast is above about 120 mg/dL. In some embodiments, when the fast lasts about 8 to about 16 hours, the individual exhibits abnormal blood glucose control if the blood glucose level of the individual after the fast is above about 119 mg/dL. In some embodiments, when the fast lasts about 8 to about 16 hours, the individual exhibits abnormal blood glucose control if the blood glucose level of the individual after the fast is above about 118 mg/dL. In some embodiments, when the fast lasts about 8 to about 16 hours, the individual exhibits abnormal blood glucose control if the blood glucose level of the individual after the fast is above about 117 mg/dL. In some embodiments, when the fast lasts about 8 to about 16 hours, the individual exhibits abnormal blood glucose control if the blood glucose level of the individual after the fast is above about 116 mg/dL. In some embodiments, when the fast lasts about 8 to about 16 hours, the individual exhibits abnormal blood glucose control if the blood glucose level of the individual after the fast is above about 115 mg/dL. In some embodiments, when the fast lasts about 8 to about 16 hours, the individual exhibits abnormal blood glucose control if the blood glucose level of the individual after the fast is above about 114 mg/dL. In some embodiments, when the fast lasts about 8 to about 16 hours, the individual exhibits abnormal blood glucose control if the blood glucose level of the individual after the fast is above about 113 mg/dL. In some embodiments, when the fast lasts about 8 to about 16 hours, the individual exhibits abnormal blood glucose control if the blood glucose level of the individual after the fast is above about 112 mg/dL. In some embodiments, when the fast lasts about 8 to about 16 hours, the individual exhibits abnormal blood glucose control if the blood glucose level of the individual after the fast is above about 111 mg/dL. In some embodiments, when the fast lasts about 8 to about 16 hours, the individual exhibits abnormal blood glucose control if the blood glucose level of the individual after the fast is above about 110 mg/dL. In some embodiments, when the fast lasts about 8 to about 16 hours, the individual exhibits abnormal blood glucose control if the blood glucose level of the individual after the fast is above about 109 mg/dL. In some embodiments, when the fast lasts about 8 to about 16 hours, the individual exhibits abnormal blood glucose control if the blood glucose level of the individual after the fast is above about 108 mg/dL. In some embodiments, when the fast lasts about 8 to about 16 hours, the individual exhibits abnormal blood glucose control if the blood glucose level of the individual after the fast is above about 107 mg/dL. In some embodiments, when the fast lasts about 8 to about 16 hours, the individual exhibits abnormal blood glucose control if the blood glucose level of the individual after the fast is above about 106 mg/dL. In some embodiments, when the fast lasts about 8 to about 16 hours, the individual exhibits abnormal blood glucose control if the blood glucose level of the individual after the fast is above about 105 mg/dL. In some embodiments, when the fast lasts about 8 to about 16 hours, the individual exhibits abnormal blood glucose control if the blood glucose level of the individual after the fast is above about 104 mg/dL. In some embodiments, when the fast lasts about 8 to about 16 hours, the individual exhibits abnormal blood glucose control if the blood glucose level of the individual after the fast is above about 103 mg/dL. In some embodiments, when the fast lasts about 8 to about 16 hours, the individual exhibits abnormal blood glucose control if the blood glucose level of the individual after the fast is above about 102 mg/dL. In some embodiments, when the fast lasts about 8 to about 16 hours, the individual exhibits abnormal blood glucose control if the blood glucose level of the individual after the fast is above about 101 mg/dL. In some embodiments, when the fast lasts about 8 to about 16 hours, the individual exhibits abnormal blood glucose control if the blood glucose level of the individual after the fast is above about 100 mg/dL. In some embodiments, when the fast lasts about 8 to about 16 hours, the individual exhibits abnormal blood glucose control if the blood glucose level of the individual after the fast is above about 99 mg/dL. In some embodiments, when the fast lasts about 8 to about 16 hours, the individual exhibits abnormal blood glucose control if the blood glucose level of the individual after the fast is above about 98 mg/dL. In some embodiments, when the fast lasts about 8 to about 16 hours, the individual exhibits abnormal blood glucose control if the blood glucose level of the individual after the fast is above about 97 mg/dL. In some embodiments, when the fast lasts about 8 to about 16 hours, the individual exhibits abnormal blood glucose control if the blood glucose level of the individual after the fast is above about 96 mg/dL. In some embodiments, when the fast lasts about 8 to about 16 hours, the individual exhibits abnormal blood glucose control if the blood glucose level of the individual after the fast is above about 95 mg/dL. In some embodiments, when the fast lasts about 8 to about 16 hours, the individual exhibits abnormal blood glucose control if the blood glucose level of the individual after the fast is above about 94 mg/dL. In some embodiments, when the fast lasts about 8 to about 16 hours, the individual exhibits abnormal blood glucose control if the blood glucose level of the individual after the fast is above about 93 mg/dL. In some embodiments, when the fast lasts about 8 to about 16 hours, the individual exhibits abnormal blood glucose control if the blood glucose level of the individual after the fast is above about 92 mg/dL. In some embodiments, when the fast lasts about 8 to about 16 hours, the individual exhibits abnormal blood glucose control if the blood glucose level of the individual after the fast is above about 91 mg/dL. In some embodiments, when the fast lasts about 8 to about 16 hours, the individual exhibits abnormal blood glucose control if the blood glucose level of the individual after the fast is above about 90 mg/dL. In some embodiments, when the fast lasts about 8 to about 16 hours, the individual exhibits abnormal blood glucose control if the blood glucose level of the individual after the fast is above about 89 mg/dL. In some embodiments, when the fast lasts about 8 to about 16 hours, the individual exhibits abnormal blood glucose control if the blood glucose level of the individual after the fast is above about 88 mg/dL. In some embodiments, when the fast lasts about 8 to about 16 hours, the individual exhibits abnormal blood glucose control if the blood glucose level of the individual after the fast is above about 87 mg/dL. In some embodiments, when the fast lasts about 8 to about 16 hours, the individual exhibits abnormal blood glucose control if the blood glucose level of the individual after the fast is above about 86 mg/dL. In some embodiments, when the fast lasts about 8 to about 16 hours, the individual exhibits abnormal blood glucose control if the blood glucose level of the individual after the fast is above about 85 mg/dL. In some embodiments, when the fast lasts about 8 to about 16 hours, the individual exhibits abnormal blood glucose control if the blood glucose level of the individual after the fast is above about 84 mg/dL. In some embodiments, when the fast lasts about 8 to about 16 hours, the individual exhibits abnormal blood glucose control if the blood glucose level of the individual after the fast is above about 83 mg/dL. In some embodiments, when the fast lasts about 8 to about 16 hours, the individual exhibits abnormal blood glucose control if the blood glucose level of the individual after the fast is above about 82 mg/dL. In some embodiments, when the fast lasts about 8 to about 16 hours, the individual exhibits abnormal blood glucose control if the blood glucose level of the individual after the fast is above about 81 mg/dL. In some embodiments, when the fast lasts about 8 to about 16 hours, the individual exhibits abnormal blood glucose control if the blood glucose level of the individual after the fast is above about 80 mg/dL. In some embodiments, when the fast lasts about 8 to about 16 hours, the individual exhibits abnormal blood glucose control if the blood glucose level of the individual after the fast is above about 79 mg/dL. In some embodiments, when the fast lasts about 8 to about 16 hours, the individual exhibits abnormal blood glucose control if the blood glucose level of the individual after the fast is above about 78 mg/dL. In some embodiments, when the fast lasts about 8 to about 16 hours, the individual exhibits abnormal blood glucose control if the blood glucose level of the individual after the fast is above about 77 mg/dL. In some embodiments, when the fast lasts about 8 to about 16 hours, the individual exhibits abnormal blood glucose control if the blood glucose level of the individual after the fast is above about 76 mg/dL. In some embodiments, when the fast lasts about 8 to about 16 hours, the individual exhibits abnormal blood glucose control if the blood glucose level of the individual after the fast is above about 75 mg/dL.

In some embodiments, when the fast lasts about 8 hours to about 16 hours, the individual exhibits abnormal blood glucose control if the blood glucose level of the individual after the fast is above about 75 mg/dL, e.g., about 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, or about 150 mg/dL, or any concentration above 150 mg/dL.

In some embodiments, the test for blood glucose control comprises a measurement of non-fasting blood glucose. In some embodiments, the measurement of non-fasting blood glucose comprises measuring a blood glucose level or a blood sugar level of an individual without subjecting the individual to fasting. In some embodiments, the measurement of non-fasting blood glucose comprises measuring a blood glucose level or a blood sugar level of an individual at any time without subjecting the individual to fasting.

In some embodiments, the individual exhibits normal blood glucose control if the individual exhibits a non-fasting blood glucose level below about 250 mg/dL. In some embodiments, the individual exhibits normal blood glucose control if the individual exhibits a non-fasting blood glucose level below about 249 mg/dL. In some embodiments, the individual exhibits normal blood glucose control if the individual exhibits a non-fasting blood glucose level below about 248 mg/dL. In some embodiments, the individual exhibits normal blood glucose control if the individual exhibits a non-fasting blood glucose level below about 247 mg/dL. In some embodiments, the individual exhibits normal blood glucose control if the individual exhibits a non-fasting blood glucose level below about 246 mg/dL. In some embodiments, the individual exhibits normal blood glucose control if the individual exhibits a non-fasting blood glucose level below about 245 mg/dL. In some embodiments, the individual exhibits normal blood glucose control if the individual exhibits a non-fasting blood glucose level below about 244 mg/dL. In some embodiments, the individual exhibits normal blood glucose control if the individual exhibits a non-fasting blood glucose level below about 243 mg/dL. In some embodiments, the individual exhibits normal blood glucose control if the individual exhibits a non-fasting blood glucose level below about 242 mg/dL. In some embodiments, the individual exhibits normal blood glucose control if the individual exhibits a non-fasting blood glucose level below about 241 mg/dL. In some embodiments, the individual exhibits normal blood glucose control if the individual exhibits a non-fasting blood glucose level below about 240 mg/dL. In some embodiments, the individual exhibits normal blood glucose control if the individual exhibits a non-fasting blood glucose level below about 239 mg/dL. In some embodiments, the individual exhibits normal blood glucose control if the individual exhibits a non-fasting blood glucose level below about 238 mg/dL. In some embodiments, the individual exhibits normal blood glucose control if the individual exhibits a non-fasting blood glucose level below about 237 mg/dL. In some embodiments, the individual exhibits normal blood glucose control if the individual exhibits a non-fasting blood glucose level below about 236 mg/dL. In some embodiments, the individual exhibits normal blood glucose control if the individual exhibits a non-fasting blood glucose level below about 235 mg/dL. In some embodiments, the individual exhibits normal blood glucose control if the individual exhibits a non-fasting blood glucose level below about 234 mg/dL. In some embodiments, the individual exhibits normal blood glucose control if the individual exhibits a non-fasting blood glucose level below about 233 mg/dL. In some embodiments, the individual exhibits normal blood glucose control if the individual exhibits a non-fasting blood glucose level below about 232 mg/dL. In some embodiments, the individual exhibits normal blood glucose control if the individual exhibits a non-fasting blood glucose level below about 231 mg/dL. In some embodiments, the individual exhibits normal blood glucose control if the individual exhibits a non-fasting blood glucose level below about 230 mg/dL. In some embodiments, the individual exhibits normal blood glucose control if the individual exhibits a non-fasting blood glucose level below about 229 mg/dL. In some embodiments, the individual exhibits normal blood glucose control if the individual exhibits a non-fasting blood glucose level below about 228 mg/dL. In some embodiments, the individual exhibits normal blood glucose control if the individual exhibits a non-fasting blood glucose level below about 227 mg/dL. In some embodiments, the individual exhibits normal blood glucose control if the individual exhibits a non-fasting blood glucose level below about 226 mg/dL. In some embodiments, the individual exhibits normal blood glucose control if the individual exhibits a non-fasting blood glucose level below about 225 mg/dL. In some embodiments, the individual exhibits normal blood glucose control if the individual exhibits a non-fasting blood glucose level below about 224 mg/dL. In some embodiments, the individual exhibits normal blood glucose control if the individual exhibits a non-fasting blood glucose level below about 223 mg/dL. In some embodiments, the individual exhibits normal blood glucose control if the individual exhibits a non-fasting blood glucose level below about 222 mg/dL. In some embodiments, the individual exhibits normal blood glucose control if the individual exhibits a non-fasting blood glucose level below about 221 mg/dL. In some embodiments, the individual exhibits normal blood glucose control if the individual exhibits a non-fasting blood glucose level below about 220 mg/dL. In some embodiments, the individual exhibits normal blood glucose control if the individual exhibits a non-fasting blood glucose level below about 219 mg/dL. In some embodiments, the individual exhibits normal blood glucose control if the individual exhibits a non-fasting blood glucose level below about 218 mg/dL. In some embodiments, the individual exhibits normal blood glucose control if the individual exhibits a non-fasting blood glucose level below about 217 mg/dL. In some embodiments, the individual exhibits normal blood glucose control if the individual exhibits a non-fasting blood glucose level below about 216 mg/dL. In some embodiments, the individual exhibits normal blood glucose control if the individual exhibits a non-fasting blood glucose level below about 215 mg/dL. In some embodiments, the individual exhibits normal blood glucose control if the individual exhibits a non-fasting blood glucose level below about 214 mg/dL. In some embodiments, the individual exhibits normal blood glucose control if the individual exhibits a non-fasting blood glucose level below about 213 mg/dL. In some embodiments, the individual exhibits normal blood glucose control if the individual exhibits a non-fasting blood glucose level below about 212 mg/dL. In some embodiments, the individual exhibits normal blood glucose control if the individual exhibits a non-fasting blood glucose level below about 211 mg/dL. In some embodiments, the individual exhibits normal blood glucose control if the individual exhibits a non-fasting blood glucose level below about 210 mg/dL. In some embodiments, the individual exhibits normal blood glucose control if the individual exhibits a non-fasting blood glucose level below about 209 mg/dL. In some embodiments, the individual exhibits normal blood glucose control if the individual exhibits a non-fasting blood glucose level below about 208 mg/dL. In some embodiments, the individual exhibits normal blood glucose control if the individual exhibits a non-fasting blood glucose level below about 207 mg/dL. In some embodiments, the individual exhibits normal blood glucose control if the individual exhibits a non-fasting blood glucose level below about 206 mg/dL. In some embodiments, the individual exhibits normal blood glucose control if the individual exhibits a non-fasting blood glucose level below about 205 mg/dL. In some embodiments, the individual exhibits normal blood glucose control if the individual exhibits a non-fasting blood glucose level below about 204 mg/dL. In some embodiments, the individual exhibits normal blood glucose control if the individual exhibits a non-fasting blood glucose level below about 203 mg/dL. In some embodiments, the individual exhibits normal blood glucose control if the individual exhibits a non-fasting blood glucose level below about 202 mg/dL. In some embodiments, the individual exhibits normal blood glucose control if the individual exhibits a non-fasting blood glucose level below about 201 mg/dL. In some embodiments, the individual exhibits normal blood glucose control if the individual exhibits a non-fasting blood glucose level below about 200 mg/dL. In some embodiments, the individual exhibits normal blood glucose control if the individual exhibits a non-fasting blood glucose level below about 199 mg/dL. In some embodiments, the individual exhibits normal blood glucose control if the individual exhibits a non-fasting blood glucose level below about 198 mg/dL. In some embodiments, the individual exhibits normal blood glucose control if the individual exhibits a non-fasting blood glucose level below about 197 mg/dL. In some embodiments, the individual exhibits normal blood glucose control if the individual exhibits a non-fasting blood glucose level below about 196 mg/dL. In some embodiments, the individual exhibits normal blood glucose control if the individual exhibits a non-fasting blood glucose level below about 195 mg/dL. In some embodiments, the individual exhibits normal blood glucose control if the individual exhibits a non-fasting blood glucose level below about 194 mg/dL. In some embodiments, the individual exhibits normal blood glucose control if the individual exhibits a non-fasting blood glucose level below about 193 mg/dL. In some embodiments, the individual exhibits normal blood glucose control if the individual exhibits a non-fasting blood glucose level below about 192 mg/dL. In some embodiments, the individual exhibits normal blood glucose control if the individual exhibits a non-fasting blood glucose level below about 191 mg/dL. In some embodiments, the individual exhibits normal blood glucose control if the individual exhibits a non-fasting blood glucose level below about 190 mg/dL. In some embodiments, the individual exhibits normal blood glucose control if the individual exhibits a non-fasting blood glucose level below about 189 mg/dL. In some embodiments, the individual exhibits normal blood glucose control if the individual exhibits a non-fasting blood glucose level below about 188 mg/dL. In some embodiments, the individual exhibits normal blood glucose control if the individual exhibits a non-fasting blood glucose level below about 187 mg/dL. In some embodiments, the individual exhibits normal blood glucose control if the individual exhibits a non-fasting blood glucose level below about 186 mg/dL. In some embodiments, the individual exhibits normal blood glucose control if the individual exhibits a non-fasting blood glucose level below about 185 mg/dL. In some embodiments, the individual exhibits normal blood glucose control if the individual exhibits a non-fasting blood glucose level below about 184 mg/dL. In some embodiments, the individual exhibits normal blood glucose control if the individual exhibits a non-fasting blood glucose level below about 183 mg/dL. In some embodiments, the individual exhibits normal blood glucose control if the individual exhibits a non-fasting blood glucose level below about 182 mg/dL. In some embodiments, the individual exhibits normal blood glucose control if the individual exhibits a non-fasting blood glucose level below about 181 mg/dL. In some embodiments, the individual exhibits normal blood glucose control if the individual exhibits a non-fasting blood glucose level below about 180 mg/dL. In some embodiments, the individual exhibits normal blood glucose control if the individual exhibits a non-fasting blood glucose level below about 179 mg/dL. In some embodiments, the individual exhibits normal blood glucose control if the individual exhibits a non-fasting blood glucose level below about 178 mg/dL. In some embodiments, the individual exhibits normal blood glucose control if the individual exhibits a non-fasting blood glucose level below about 177 mg/dL. In some embodiments, the individual exhibits normal blood glucose control if the individual exhibits a non-fasting blood glucose level below about 176 mg/dL. In some embodiments, the individual exhibits normal blood glucose control if the individual exhibits a non-fasting blood glucose level below about 175 mg/dL. In some embodiments, the individual exhibits normal blood glucose control if the individual exhibits a non-fasting blood glucose level below about 174 mg/dL. In some embodiments, the individual exhibits normal blood glucose control if the individual exhibits a non-fasting blood glucose level below about 173 mg/dL. In some embodiments, the individual exhibits normal blood glucose control if the individual exhibits a non-fasting blood glucose level below about 172 mg/dL. In some embodiments, the individual exhibits normal blood glucose control if the individual exhibits a non-fasting blood glucose level below about 171 mg/dL. In some embodiments, the individual exhibits normal blood glucose control if the individual exhibits a non-fasting blood glucose level below about 170 mg/dL. In some embodiments, the individual exhibits normal blood glucose control if the individual exhibits a non-fasting blood glucose level below about 169 mg/dL. In some embodiments, the individual exhibits normal blood glucose control if the individual exhibits a non-fasting blood glucose level below about 168 mg/dL. In some embodiments, the individual exhibits normal blood glucose control if the individual exhibits a non-fasting blood glucose level below about 167 mg/dL. In some embodiments, the individual exhibits normal blood glucose control if the individual exhibits a non-fasting blood glucose level below about 166 mg/dL. In some embodiments, the individual exhibits normal blood glucose control if the individual exhibits a non-fasting blood glucose level below about 165 mg/dL. In some embodiments, the individual exhibits normal blood glucose control if the individual exhibits a non-fasting blood glucose level below about 164 mg/dL. In some embodiments, the individual exhibits normal blood glucose control if the individual exhibits a non-fasting blood glucose level below about 163 mg/dL. In some embodiments, the individual exhibits normal blood glucose control if the individual exhibits a non-fasting blood glucose level below about 162 mg/dL. In some embodiments, the individual exhibits normal blood glucose control if the individual exhibits a non-fasting blood glucose level below about 161 mg/dL. In some embodiments, the individual exhibits normal blood glucose control if the individual exhibits a non-fasting blood glucose level below about 160 mg/dL. In some embodiments, the individual exhibits normal blood glucose control if the individual exhibits a non-fasting blood glucose level below about 159 mg/dL. In some embodiments, the individual exhibits normal blood glucose control if the individual exhibits a non-fasting blood glucose level below about 158 mg/dL. In some embodiments, the individual exhibits normal blood glucose control if the individual exhibits a non-fasting blood glucose level below about 157 mg/dL. In some embodiments, the individual exhibits normal blood glucose control if the individual exhibits a non-fasting blood glucose level below about 156 mg/dL. In some embodiments, the individual exhibits normal blood glucose control if the individual exhibits a non-fasting blood glucose level below about 155 mg/dL. In some embodiments, the individual exhibits normal blood glucose control if the individual exhibits a non-fasting blood glucose level below about 154 mg/dL. In some embodiments, the individual exhibits normal blood glucose control if the individual exhibits a non-fasting blood glucose level below about 153 mg/dL. In some embodiments, the individual exhibits normal blood glucose control if the individual exhibits a non-fasting blood glucose level below about 152 mg/dL. In some embodiments, the individual exhibits normal blood glucose control if the individual exhibits a non-fasting blood glucose level below about 151 mg/dL. In some embodiments, the individual exhibits normal blood glucose control if the individual exhibits a non-fasting blood glucose level below about 150 mg/dL.

In some embodiments, the individual exhibits normal blood glucose control if the individual exhibits a non-fasting blood glucose level from about 0 to about 250 mg/dL, e.g., about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, or about 250 mg/dL, or any concentration therebetween.

In some embodiments, the individual exhibits abnormal blood glucose control if the individual exhibits a non-fasting blood glucose level of at least about 250 mg/dL. In some embodiments, the individual exhibits abnormal blood glucose control if the individual exhibits a non-fasting blood glucose level of at least about 249 mg/dL. In some embodiments, the individual exhibits abnormal blood glucose control if the individual exhibits a non-fasting blood glucose level of at least about 248 mg/dL. In some embodiments, the individual exhibits abnormal blood glucose control if the individual exhibits a non-fasting blood glucose level of at least about 247 mg/dL. In some embodiments, the individual exhibits abnormal blood glucose control if the individual exhibits a non-fasting blood glucose level of at least about 246 mg/dL. In some embodiments, the individual exhibits abnormal blood glucose control if the individual exhibits a non-fasting blood glucose level of at least about 245 mg/dL. In some embodiments, the individual exhibits abnormal blood glucose control if the individual exhibits a non-fasting blood glucose level of at least about 244 mg/dL. In some embodiments, the individual exhibits abnormal blood glucose control if the individual exhibits a non-fasting blood glucose level of at least about 243 mg/dL. In some embodiments, the individual exhibits abnormal blood glucose control if the individual exhibits a non-fasting blood glucose level of at least about 242 mg/dL. In some embodiments, the individual exhibits abnormal blood glucose control if the individual exhibits a non-fasting blood glucose level of at least about 241 mg/dL. In some embodiments, the individual exhibits abnormal blood glucose control if the individual exhibits a non-fasting blood glucose level of at least about 240 mg/dL. In some embodiments, the individual exhibits abnormal blood glucose control if the individual exhibits a non-fasting blood glucose level of at least about 239 mg/dL. In some embodiments, the individual exhibits abnormal blood glucose control if the individual exhibits a non-fasting blood glucose level of at least about 238 mg/dL. In some embodiments, the individual exhibits abnormal blood glucose control if the individual exhibits a non-fasting blood glucose level of at least about 237 mg/dL. In some embodiments, the individual exhibits abnormal blood glucose control if the individual exhibits a non-fasting blood glucose level of at least about 236 mg/dL. In some embodiments, the individual exhibits abnormal blood glucose control if the individual exhibits a non-fasting blood glucose level of at least about 235 mg/dL. In some embodiments, the individual exhibits abnormal blood glucose control if the individual exhibits a non-fasting blood glucose level of at least about 234 mg/dL. In some embodiments, the individual exhibits abnormal blood glucose control if the individual exhibits a non-fasting blood glucose level of at least about 233 mg/dL. In some embodiments, the individual exhibits abnormal blood glucose control if the individual exhibits a non-fasting blood glucose level of at least about 232 mg/dL. In some embodiments, the individual exhibits abnormal blood glucose control if the individual exhibits a non-fasting blood glucose level of at least about 231 mg/dL. In some embodiments, the individual exhibits abnormal blood glucose control if the individual exhibits a non-fasting blood glucose level of at least about 230 mg/dL. In some embodiments, the individual exhibits abnormal blood glucose control if the individual exhibits a non-fasting blood glucose level of at least about 229 mg/dL. In some embodiments, the individual exhibits abnormal blood glucose control if the individual exhibits a non-fasting blood glucose level of at least about 228 mg/dL. In some embodiments, the individual exhibits abnormal blood glucose control if the individual exhibits a non-fasting blood glucose level of at least about 227 mg/dL. In some embodiments, the individual exhibits abnormal blood glucose control if the individual exhibits a non-fasting blood glucose level of at least about 226 mg/dL. In some embodiments, the individual exhibits abnormal blood glucose control if the individual exhibits a non-fasting blood glucose level of at least about 225 mg/dL. In some embodiments, the individual exhibits abnormal blood glucose control if the individual exhibits a non-fasting blood glucose level of at least about 224 mg/dL. In some embodiments, the individual exhibits abnormal blood glucose control if the individual exhibits a non-fasting blood glucose level of at least about 223 mg/dL. In some embodiments, the individual exhibits abnormal blood glucose control if the individual exhibits a non-fasting blood glucose level of at least about 222 mg/dL. In some embodiments, the individual exhibits abnormal blood glucose control if the individual exhibits a non-fasting blood glucose level of at least about 221 mg/dL. In some embodiments, the individual exhibits abnormal blood glucose control if the individual exhibits a non-fasting blood glucose level of at least about 220 mg/dL. In some embodiments, the individual exhibits abnormal blood glucose control if the individual exhibits a non-fasting blood glucose level of at least about 219 mg/dL. In some embodiments, the individual exhibits abnormal blood glucose control if the individual exhibits a non-fasting blood glucose level of at least about 218 mg/dL. In some embodiments, the individual exhibits abnormal blood glucose control if the individual exhibits a non-fasting blood glucose level of at least about 217 mg/dL. In some embodiments, the individual exhibits abnormal blood glucose control if the individual exhibits a non-fasting blood glucose level of at least about 216 mg/dL. In some embodiments, the individual exhibits abnormal blood glucose control if the individual exhibits a non-fasting blood glucose level of at least about 215 mg/dL. In some embodiments, the individual exhibits abnormal blood glucose control if the individual exhibits a non-fasting blood glucose level of at least about 214 mg/dL. In some embodiments, the individual exhibits abnormal blood glucose control if the individual exhibits a non-fasting blood glucose level of at least about 213 mg/dL. In some embodiments, the individual exhibits abnormal blood glucose control if the individual exhibits a non-fasting blood glucose level of at least about 212 mg/dL. In some embodiments, the individual exhibits abnormal blood glucose control if the individual exhibits a non-fasting blood glucose level of at least about 211 mg/dL. In some embodiments, the individual exhibits abnormal blood glucose control if the individual exhibits a non-fasting blood glucose level of at least about 210 mg/dL. In some embodiments, the individual exhibits abnormal blood glucose control if the individual exhibits a non-fasting blood glucose level of at least about 209 mg/dL. In some embodiments, the individual exhibits abnormal blood glucose control if the individual exhibits a non-fasting blood glucose level of at least about 208 mg/dL. In some embodiments, the individual exhibits abnormal blood glucose control if the individual exhibits a non-fasting blood glucose level of at least about 207 mg/dL. In some embodiments, the individual exhibits abnormal blood glucose control if the individual exhibits a non-fasting blood glucose level of at least about 206 mg/dL. In some embodiments, the individual exhibits abnormal blood glucose control if the individual exhibits a non-fasting blood glucose level of at least about 205 mg/dL. In some embodiments, the individual exhibits abnormal blood glucose control if the individual exhibits a non-fasting blood glucose level of at least about 204 mg/dL. In some embodiments, the individual exhibits abnormal blood glucose control if the individual exhibits a non-fasting blood glucose level of at least about 203 mg/dL. In some embodiments, the individual exhibits abnormal blood glucose control if the individual exhibits a non-fasting blood glucose level of at least about 202 mg/dL. In some embodiments, the individual exhibits abnormal blood glucose control if the individual exhibits a non-fasting blood glucose level of at least about 201 mg/dL. In some embodiments, the individual exhibits abnormal blood glucose control if the individual exhibits a non-fasting blood glucose level of at least about 200 mg/dL. In some embodiments, the individual exhibits abnormal blood glucose control if the individual exhibits a non-fasting blood glucose level of at least about 199 mg/dL. In some embodiments, the individual exhibits abnormal blood glucose control if the individual exhibits a non-fasting blood glucose level of at least about 198 mg/dL. In some embodiments, the individual exhibits abnormal blood glucose control if the individual exhibits a non-fasting blood glucose level of at least about 197 mg/dL. In some embodiments, the individual exhibits abnormal blood glucose control if the individual exhibits a non-fasting blood glucose level of at least about 196 mg/dL. In some embodiments, the individual exhibits abnormal blood glucose control if the individual exhibits a non-fasting blood glucose level of at least about 195 mg/dL. In some embodiments, the individual exhibits abnormal blood glucose control if the individual exhibits a non-fasting blood glucose level of at least about 194 mg/dL. In some embodiments, the individual exhibits abnormal blood glucose control if the individual exhibits a non-fasting blood glucose level of at least about 193 mg/dL. In some embodiments, the individual exhibits abnormal blood glucose control if the individual exhibits a non-fasting blood glucose level of at least about 192 mg/dL. In some embodiments, the individual exhibits abnormal blood glucose control if the individual exhibits a non-fasting blood glucose level of at least about 191 mg/dL. In some embodiments, the individual exhibits abnormal blood glucose control if the individual exhibits a non-fasting blood glucose level of at least about 190 mg/dL. In some embodiments, the individual exhibits abnormal blood glucose control if the individual exhibits a non-fasting blood glucose level of at least about 189 mg/dL. In some embodiments, the individual exhibits abnormal blood glucose control if the individual exhibits a non-fasting blood glucose level of at least about 188 mg/dL. In some embodiments, the individual exhibits abnormal blood glucose control if the individual exhibits a non-fasting blood glucose level of at least about 187 mg/dL. In some embodiments, the individual exhibits abnormal blood glucose control if the individual exhibits a non-fasting blood glucose level of at least about 186 mg/dL. In some embodiments, the individual exhibits abnormal blood glucose control if the individual exhibits a non-fasting blood glucose level of at least about 185 mg/dL. In some embodiments, the individual exhibits abnormal blood glucose control if the individual exhibits a non-fasting blood glucose level of at least about 184 mg/dL. In some embodiments, the individual exhibits abnormal blood glucose control if the individual exhibits a non-fasting blood glucose level of at least about 183 mg/dL. In some embodiments, the individual exhibits abnormal blood glucose control if the individual exhibits a non-fasting blood glucose level of at least about 182 mg/dL. In some embodiments, the individual exhibits abnormal blood glucose control if the individual exhibits a non-fasting blood glucose level of at least about 181 mg/dL. In some embodiments, the individual exhibits abnormal blood glucose control if the individual exhibits a non-fasting blood glucose level of at least about 180 mg/dL. In some embodiments, the individual exhibits abnormal blood glucose control if the individual exhibits a non-fasting blood glucose level of at least about 179 mg/dL. In some embodiments, the individual exhibits abnormal blood glucose control if the individual exhibits a non-fasting blood glucose level of at least about 178 mg/dL. In some embodiments, the individual exhibits abnormal blood glucose control if the individual exhibits a non-fasting blood glucose level of at least about 177 mg/dL. In some embodiments, the individual exhibits abnormal blood glucose control if the individual exhibits a non-fasting blood glucose level of at least about 176 mg/dL. In some embodiments, the individual exhibits abnormal blood glucose control if the individual exhibits a non-fasting blood glucose level of at least about 175 mg/dL. In some embodiments, the individual exhibits abnormal blood glucose control if the individual exhibits a non-fasting blood glucose level of at least about 174 mg/dL. In some embodiments, the individual exhibits abnormal blood glucose control if the individual exhibits a non-fasting blood glucose level of at least about 173 mg/dL. In some embodiments, the individual exhibits abnormal blood glucose control if the individual exhibits a non-fasting blood glucose level of at least about 172 mg/dL. In some embodiments, the individual exhibits abnormal blood glucose control if the individual exhibits a non-fasting blood glucose level of at least about 171 mg/dL. In some embodiments, the individual exhibits abnormal blood glucose control if the individual exhibits a non-fasting blood glucose level of at least about 170 mg/dL. In some embodiments, the individual exhibits abnormal blood glucose control if the individual exhibits a non-fasting blood glucose level of at least about 169 mg/dL. In some embodiments, the individual exhibits abnormal blood glucose control if the individual exhibits a non-fasting blood glucose level of at least about 168 mg/dL. In some embodiments, the individual exhibits abnormal blood glucose control if the individual exhibits a non-fasting blood glucose level of at least about 167 mg/dL. In some embodiments, the individual exhibits abnormal blood glucose control if the individual exhibits a non-fasting blood glucose level of at least about 166 mg/dL. In some embodiments, the individual exhibits abnormal blood glucose control if the individual exhibits a non-fasting blood glucose level of at least about 165 mg/dL. In some embodiments, the individual exhibits abnormal blood glucose control if the individual exhibits a non-fasting blood glucose level of at least about 164 mg/dL. In some embodiments, the individual exhibits abnormal blood glucose control if the individual exhibits a non-fasting blood glucose level of at least about 163 mg/dL. In some embodiments, the individual exhibits abnormal blood glucose control if the individual exhibits a non-fasting blood glucose level of at least about 162 mg/dL. In some embodiments, the individual exhibits abnormal blood glucose control if the individual exhibits a non-fasting blood glucose level of at least about 161 mg/dL. In some embodiments, the individual exhibits abnormal blood glucose control if the individual exhibits a non-fasting blood glucose level of at least about 160 mg/dL. In some embodiments, the individual exhibits abnormal blood glucose control if the individual exhibits a non-fasting blood glucose level of at least about 159 mg/dL. In some embodiments, the individual exhibits abnormal blood glucose control if the individual exhibits a non-fasting blood glucose level of at least about 158 mg/dL. In some embodiments, the individual exhibits abnormal blood glucose control if the individual exhibits a non-fasting blood glucose level of at least about 157 mg/dL. In some embodiments, the individual exhibits abnormal blood glucose control if the individual exhibits a non-fasting blood glucose level of at least about 156 mg/dL. In some embodiments, the individual exhibits abnormal blood glucose control if the individual exhibits a non-fasting blood glucose level of at least about 155 mg/dL. In some embodiments, the individual exhibits abnormal blood glucose control if the individual exhibits a non-fasting blood glucose level of at least about 154 mg/dL. In some embodiments, the individual exhibits abnormal blood glucose control if the individual exhibits a non-fasting blood glucose level of at least about 153 mg/dL. In some embodiments, the individual exhibits abnormal blood glucose control if the individual exhibits a non-fasting blood glucose level of at least about 152 mg/dL. In some embodiments, the individual exhibits abnormal blood glucose control if the individual exhibits a non-fasting blood glucose level of at least about 151 mg/dL. In some embodiments, the individual exhibits abnormal blood glucose control if the individual exhibits a non-fasting blood glucose level of at least about 150 mg/dL.

In some embodiments, the individual exhibits abnormal blood glucose control if the individual exhibits a non-fasting blood glucose level of at least about 150 mg/dL, e.g., about 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, 251 mg/dL, 252 mg/dL, 253 mg/dL, 254 mg/dL, 255 mg/dL, 256 mg/dL, 257 mg/dL, 258 mg/dL, 259 mg/dL, 260 mg/dL, 261 mg/dL, 262 mg/dL, 263 mg/dL, 264 mg/dL, 265 mg/dL, 266 mg/dL, 267 mg/dL, 268 mg/dL, 269 mg/dL, 270 mg/dL, 271 mg/dL, 272 mg/dL, 273 mg/dL, 274 mg/dL, 275 mg/dL, 276 mg/dL, 277 mg/dL, 278 mg/dL, 279 mg/dL, 280 mg/dL, 281 mg/dL, 282 mg/dL, 283 mg/dL, 284 mg/dL, 285 mg/dL, 286 mg/dL, 287 mg/dL, 288 mg/dL, 289 mg/dL, 290 mg/dL, 291 mg/dL, 292 mg/dL, 293 mg/dL, 294 mg/dL, 295 mg/dL, 296 mg/dL, 297 mg/dL, 298 mg/dL, 299 mg/dL, or about 300 mg/dL, or any concentration above about 300 mg/dL.

In some embodiments, the individual is diagnosed with a disorder associated with high blood glucose before or during the treatment with the antagonist of IGF1R signaling. In some embodiments, before or during the treatment with the antagonist of IGF1R signaling, the individual is diagnosed with one or more selected from the group comprising hyperglycemia, prediabetes, diabetes, gestational diabetes, type 1 diabetes, type 2 diabetes, glucose intolerance, impaired fasting glucose, or impaired glucose tolerance. Hyperglycemia is the technical term for high blood glucose (blood sugar). High blood glucose happens when the body has too little insulin or when the body can't use insulin properly.

Diabetes is a chronic, metabolic disease characterized by elevated levels of blood glucose (or blood sugar), which leads over time to serious damage to the heart, blood vessels, eyes, kidneys and nerves. Subjects with diabetes do not make enough insulin or cannot use insulin as well as they should. Insulin is a hormone the body uses to allow sugar (glucose) to enter cells to produce energy.

Type 1 diabetes, once known as juvenile diabetes or insulin-dependent diabetes, is a chronic condition. In this condition, the pancreas makes little or no insulin. Different factors, such as genetics and some viruses, may cause type 1 diabetes. Although type 1 diabetes usually appears during childhood or adolescence, it can develop in adults.

Type 2 diabetes is a condition that happens because of a problem in the way the body regulates and uses sugar as a fuel, resulting in too much sugar circulating in the blood. Eventually, high blood sugar levels can lead to disorders of the circulatory, nervous and immune systems. In type 2 diabetes, there are primarily two problems: 1) the pancreas does not produce enough insulin, and 2) cells respond poorly to insulin and take in less sugar. Type 2 diabetes used to be known as adult-onset diabetes, but both type 1 and type 2 diabetes can begin during childhood and adulthood. Type 2 is more common in older adults. But the increase in the number of children with obesity has led to more cases of type 2 diabetes in younger people. Risk factors for Type 2 diabetes include: being overweight or obese, inactivity, family history, and blood lipid levels, among other things.

Prediabetes is a health condition where blood sugar levels are higher than normal, but not high enough yet to be diagnosed as type 2 diabetes.

A fasting blood sugar level of 99 mg/dL or lower is considered normal, 100 to 125 mg/dL indicates a subject has prediabetes, and 126 mg/dL or higher indicates a subject has diabetes. A measurement of hemoglobin A1c (HbA1c) below 5.7% is considered normal, a level of 5.7% to 6.4% indicates a subject has prediabetes, and a level of greater than 6.5% indicates a subject has diabetes.

In certain embodiments, one or more anti-diabetic drugs are used to treat hyperglycemia, prediabetes, diabetes, gestational diabetes, type 1 diabetes, type 2 diabetes, glucose intolerance, impaired fasting glucose, or impaired glucose tolerance. Anti-diabetic drugs stabilize and control blood glucose amongst people with hyperglycemia or diabetes. Anti-diabetic drugs can include insulin, metformin, pioglitazone, rosiglitazone, acarbose, miglitol, bromocriptine, alogliptin, linagliptin, saxagliptin, sitagliptin, dulaglutide, exenatide, liraglutide, lixisenatide, semaglutide, tirzepatide, nateglinide, repaglinide, canagliflozin, dapagliflozin, empagliflozin, ertugliflozin, glimepiride, gliclazide, glipizide, and combinations thereof.

In some embodiments, the antagonist of IGF1R signaling or the pharmaceutical composition comprising the antagonist of IGF1R signaling may be administered to the individual through any appropriate route, including but not limited to intravenous fusion, intravenous injection, subcutaneous injection, intramuscular injection, inhalation, oral ingestion, intraperitoneal injection, intravitreal injections, subconjunctival injection, subretinal injection and topical administration. In some embodiments, teprotumumab may be administered to the individual through intravenous infusion.

In some embodiments, a dosage or a dosing regimen for the antagonist of IGF1R signaling disclosed herein is applicable to the pharmaceutical composition comprising the antagonist of IGF1R signaling which contains the same amount of the antagonist of IGF1R signaling. In some embodiments, the dosage or dosing regimen of the pharmaceutical composition can be calculated based on the effective amount of the antagonist of IGF1R signaling required in the pharmaceutical composition disclosed herein. In some embodiments, a dosage of the antagonist of IGF1R signaling or the pharmaceutical composition comprising same amount of the antagonist of IGF1R signaling may be calculated based on one or more factors selected from the group comprising the type of antagonist of IGF1R signaling, the disease or disorder being treated, physical attributes of the individual including weight, height, age, gender, medical history of the individual, delivery route, and other medical, physical or mental conditions that the individual may have, and a physician may determine an appropriate dosage to be used. In some embodiments, the dosage of the antagonist of IGF1R signaling or the pharmaceutical composition comprising the antagonist of IGF1R signaling may be altered during the course of the treatment based on the individual's response to the treatment, including but not limited to any therapeutical or adverse effect on the IGF1R associated disorder or blood glucose level. In some embodiments, different dosages of the antagonist of IGF1R signaling or the pharmaceutical composition comprising the antagonist of IGF1R signaling may be administered to the individual during the course of the treatment. In some embodiments, two or more antagonists of IGF1R signaling disclosed herein or a pharmaceutical composition comprising two or more antagonists of IGF1R signaling disclosed herein may be administered to the individual concurrently or sequentially during the course of the treatment depending on the individual's response to the treatment, including but not limited to any therapeutical or adverse effect on the IGF1R associated disorder or blood glucose level.

In some embodiments, the antagonist of IGF1R signaling comprises teprotumumab. In some embodiments, the dosage of teprotumumab may comprise a first dose. In some embodiments, the first dose of teprotumumab may be calculated based on the weight of the individual being treated. In some embodiments, the first dose of teprotumumab may be calculated based on the medical or physical conditions of the individual being treated, including but not limited to the individual's blood glucose level, conditions of the IGF1R associated disorder, and prior medical history. In some embodiments, the first dose of teprotumumab may be calculated based on any factor disclosed herein. In some embodiments, the dosage of teprotumumab may comprise a second dose. In some embodiments, the second dose of teprotumumab may be the same or different from the first dose. In some embodiments, the dosage of teprotumumab may comprise 4, 5, 6, 7, or more than 7 doses during the course of the treatment.

In some embodiments, the antagonist of IGF1R signaling may be administered at a single dosage or daily dosage of about 0.01 ng to about 1000 ng, e.g., about 0.01 ng, 0.02 ng, 0.03 ng, 0.04 ng, 0.05 ng, 0.06 ng, 0.07 ng, 0.08 ng, 0.09 ng, 0.1 ng, 0.2 ng, 0.3 ng, 0.4 ng, 0.5 ng, 0.6 ng, 0.7 ng, 0.8 ng, 0.9 ng, 1 ng, 1.1 ng, 1.2 ng, 1.3 ng, 1.4 ng, 1.5 ng, 1.6 ng, 1.7 ng, 1.8 ng, 1.9 ng, 2 ng, 2.1 ng, 2.2 ng, 2.3 ng, 2.4 ng, 2.5 ng, 2.6 ng, 2.7 ng, 2.8 ng, 2.9 ng, 3 ng, 3.1 ng, 3.2 ng, 3.3 ng, 3.4 ng, 3.5 ng, 3.6 ng, 3.7 ng, 3.8 ng, 3.9 ng, 4 ng, 4.1 ng, 4.2 ng, 4.3 ng, 4.4 ng, 4.5 ng, 4.6 ng, 4.7 ng, 4.8 ng, 4.9 ng, 5 ng, 5.1 ng, 5.2 ng, 5.3 ng, 5.4 ng, 5.5 ng, 5.6 ng, 5.7 ng, 5.8 ng, 5.9 ng, 6 ng, 6.1 ng, 6.2 ng, 6.3 ng, 6.4 ng, 6.5 ng, 6.6 ng, 6.7 ng, 6.8 ng, 6.9 ng, 7 ng, 7.1 ng, 7.2 ng, 7.3 ng, 7.4 ng, 7.5 ng, 7.6 ng, 7.7 ng, 7.8 ng, 7.9 ng, 8 ng, 8.1 ng, 8.2 ng, 8.3 ng, 8.4 ng, 8.5 ng, 8.6 ng, 8.7 ng, 8.8 ng, 8.9 ng, 9 ng, 9.1 ng, 9.2 ng, 9.3 ng, 9.4 ng, 9.5 ng, 9.6 ng, 9.7 ng, 9.8 ng, 9.9 ng, 10 ng, 10.5 ng, 11 ng, 11.5 ng, 12 ng, 12.5 ng, 13 ng, 13.5 ng, 14 ng, 14.5 ng, 15 ng, 15.5 ng, 16 ng, 16.5 ng, 17 ng, 17.5 ng, 18 ng, 18.5 ng, 19 ng, 19.5 ng, 20 ng, 20.5 ng, 21 ng, 21.5 ng, 22 ng, 22.5 ng, 23 ng, 23.5 ng, 24 ng, 24.5 ng, 25 ng, 25.5 ng, 26 ng, 26.5 ng, 27 ng, 27.5 ng, 28 ng, 28.5 ng, 29 ng, 29.5 ng, 30 ng, 30.5 ng, 31 ng, 31.5 ng, 32 ng, 32.5 ng, 33 ng, 33.5 ng, 34 ng, 34.5 ng, 35 ng, 35.5 ng, 36 ng, 36.5 ng, 37 ng, 37.5 ng, 38 ng, 38.5 ng, 39 ng, 39.5 ng, 40 ng, 40.5 ng, 41 ng, 41.5 ng, 42 ng, 42.5 ng, 43 ng, 43.5 ng, 44 ng, 44.5 ng, 45 ng, 45.5 ng, 46 ng, 46.5 ng, 47 ng, 47.5 ng, 48 ng, 48.5 ng, 49 ng, 49.5 ng, 50 ng, 51 ng, 52 ng, 53 ng, 54 ng, 55 ng, 56 ng, 57 ng, 58 ng, 59 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 100 ng, 105 ng, 110 ng, 115 ng, 120 ng, 125 ng, 130 ng, 135 ng, 140 ng, 145 ng, 150 ng, 155 ng, 160 ng, 165 ng, 170 ng, 175 ng, 180 ng, 185 ng, 190 ng, 195 ng, 200 ng, 205 ng, 210 ng, 215 ng, 220 ng, 225 ng, 230 ng, 235 ng, 240 ng, 245 ng, 250 ng, 255 ng, 260 ng, 265 ng, 270 ng, 275 ng, 280 ng, 285 ng, 290 ng, 295 ng, 300 ng, 305 ng, 310 ng, 315 ng, 320 ng, 325 ng, 330 ng, 335 ng, 340 ng, 345 ng, 350 ng, 355 ng, 360 ng, 365 ng, 370 ng, 375 ng, 380 ng, 385 ng, 390 ng, 395 ng, 400 ng, 405 ng, 410 ng, 415 ng, 420 ng, 425 ng, 430 ng, 435 ng, 440 ng, 445 ng, 450 ng, 455 ng, 460 ng, 465 ng, 470 ng, 475 ng, 480 ng, 485 ng, 490 ng, 495 ng, 500 ng, 505 ng, 510 ng, 515 ng, 520 ng, 525 ng, 530 ng, 535 ng, 540 ng, 545 ng, 550 ng, 555 ng, 560 ng, 565 ng, 570 ng, 575 ng, 580 ng, 585 ng, 590 ng, 595 ng, 600 ng, 605 ng, 610 ng, 615 ng, 620 ng, 625 ng, 630 ng, 635 ng, 640 ng, 645 ng, 650 ng, 655 ng, 660 ng, 665 ng, 670 ng, 675 ng, 680 ng, 685 ng, 690 ng, 695 ng, 700 ng, 705 ng, 710 ng, 715 ng, 720 ng, 725 ng, 730 ng, 735 ng, 740 ng, 745 ng, 750 ng, 755 ng, 760 ng, 765 ng, 770 ng, 775 ng, 780 ng, 785 ng, 790 ng, 795 ng, 800 ng, 805 ng, 810 ng, 815 ng, 820 ng, 825 ng, 830 ng, 835 ng, 840 ng, 845 ng, 850 ng, 855 ng, 860 ng, 865 ng, 870 ng, 875 ng, 880 ng, 885 ng, 890 ng, 895 ng, 900 ng, 905 ng, 910 ng, 915 ng, 920 ng, 925 ng, 930 ng, 935 ng, 940 ng, 945 ng, 950 ng, 955 ng, 960 ng, 965 ng, 970 ng, 975 ng, 980 ng, 985 ng, 990 ng, 995 ng, or about 1000 ng, or any dosage therebetween.

In some embodiments, the antagonist of IGF1R signaling may be administered at a single dosage or daily dosage of about 1 μg to about 1 mg, e.g., about 1 μg, 1.1 μg, 1.2 μg, 1.3 μg, 1.4 μg, 1.5 μg, 1.6 μg, 1.7 μg, 1.8 μg, 1.9 μg, 2 μg, 2.1 μg, 2.2 μg, 2.3 μg, 2.4 μg, 2.5 μg, 2.6 μg, 2.7 μg, 2.8 μg, 2.9 μg, 3 μg, 3.1 μg, 3.2 μg, 3.3 μg, 3.4 μg, 3.5 μg, 3.6 μg, 3.7 μg, 3.8 μg, 3.9 μg, 4 μg, 4.1 μg, 4.2 μg, 4.3 μg, 4.4 μg, 4.5 μg, 4.6 μg, 4.7 μg, 4.8 μg, 4.9 μg, 5 μg, 5.1 μg, 5.2 μg, 5.3 μg, 5.4 μg, 5.5 μg, 5.6 μg, 5.7 μg, 5.8 μg, 5.9 μg, 6 μg, 6.1 μg, 6.2 μg, 6.3 μg, 6.4 μg, 6.5 μg, 6.6 μg, 6.7 μg, 6.8 μg, 6.9 μg, 7 μg, 7.1 μg, 7.2 μg, 7.3 μg, 7.4 μg, 7.5 μg, 7.6 μg, 7.7 μg, 7.8 μg, 7.9 μg, 8 μg, 8.1 μg, 8.2 μg, 8.3 μg, 8.4 μg, 8.5 μg, 8.6 μg, 8.7 μg, 8.8 μg, 8.9 μg, 9 μg, 9.1 μg, 9.2 μg, 9.3 μg, 9.4 μg, 9.5 μg, 9.6 μg, 9.7 μg, 9.8 μg, 9.9 μg, 10 μg, 10.5 μg, 11 μg, 11.5 μg, 12 μg, 12.5 μg, 13 μg, 13.5 μg, 14 μg, 14.5 μg, 15 μg, 15.5 μg, 16 μg, 16.5 μg, 17 μg, 17.5 μg, 18 μg, 18.5 μg, 19 μg, 19.5 μg, 20 μg, 20.5 μg, 21 μg, 21.5 μg, 22 μg, 22.5 μg, 23 μg, 23.5 μg, 24 μg, 24.5 μg, 25 μg, 25.5 μg, 26 μg, 26.5 μg, 27 μg, 27.5 μg, 28 μg, 28.5 μg, 29 μg, 29.5 μg, 30 μg, 30.5 μg, 31 μg, 31.5 μg, 32 μg, 32.5 μg, 33 μg, 33.5 μg, 34 μg, 34.5 μg, 35 μg, 35.5 μg, 36 μg, 36.5 μg, 37 μg, 37.5 μg, 38 μg, 38.5 μg, 39 μg, 39.5 μg, 40 μg, 40.5 μg, 41 μg, 41.5 μg, 42 μg, 42.5 μg, 43 μg, 43.5 μg, 44 μg, 44.5 μg, 45 μg, 45.5 μg, 46 μg, 46.5 μg, 47 μg, 47.5 μg, 48 μg, 48.5 μg, 49 μg, 49.5 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, or about 1000 μg, or any dosage therebetween.

In some embodiments, the antagonist of IGF1R signaling may be administered at a single dosage or daily dosage of about 1 mg to about 1 g, e.g., about 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or about 1000 mg, or any dosage therebetween.

In some embodiments, the antagonist of IGF1R signaling may be administered at a single dosage or daily dosage of about 1 g to about 100 g, e.g., about 1 g, 1.1 g, 1.2 g, 1.3 g, 1.4 g, 1.5 g, 1.6 g, 1.7 g, 1.8 g, 1.9 g, 2 g, 2.1 g, 2.2 g, 2.3 g, 2.4 g, 2.5 g, 2.6 g, 2.7 g, 2.8 g, 2.9 g, 3 g, 3.1 g, 3.2 g, 3.3 g, 3.4 g, 3.5 g, 3.6 g, 3.7 g, 3.8 g, 3.9 g, 4 g, 4.1 g, 4.2 g, 4.3 g, 4.4 g, 4.5 g, 4.6 g, 4.7 g, 4.8 g, 4.9 g, 5 g, 5.1 g, 5.2 g, 5.3 g, 5.4 g, 5.5 g, 5.6 g, 5.7 g, 5.8 g, 5.9 g, 6 g, 6.1 g, 6.2 g, 6.3 g, 6.4 g, 6.5 g, 6.6 g, 6.7 g, 6.8 g, 6.9 g, 7 g, 7.1 g, 7.2 g, 7.3 g, 7.4 g, 7.5 g, 7.6 g, 7.7 g, 7.8 g, 7.9 g, 8 g, 8.1 g, 8.2 g, 8.3 g, 8.4 g, 8.5 g, 8.6 g, 8.7 g, 8.8 g, 8.9 g, 9 g, 9.1 g, 9.2 g, 9.3 g, 9.4 g, 9.5 g, 9.6 g, 9.7 g, 9.8 g, 9.9 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g, 18 g, 18.5 g, 19 g, 19.5 g, 20 g, 20.5 g, 21 g, 21.5 g, 22 g, 22.5 g, 23 g, 23.5 g, 24 g, 24.5 g, 25 g, 25.5 g, 26 g, 26.5 g, 27 g, 27.5 g, 28 g, 28.5 g, 29 g, 29.5 g, 30 g, 30.5 g, 31 g, 31.5 g, 32 g, 32.5 g, 33 g, 33.5 g, 34 g, 34.5 g, 35 g, 35.5 g, 36 g, 36.5 g, 37 g, 37.5 g, 38 g, 38.5 g, 39 g, 39.5 g, 40 g, 40.5 g, 41 g, 41.5 g, 42 g, 42.5 g, 43 g, 43.5 g, 44 g, 44.5 g, 45 g, 45.5 g, 46 g, 46.5 g, 47 g, 47.5 g, 48 g, 48.5 g, 49 g, 49.5 g, 50 g, 50.5 g, 51 g, 51.5 g, 52 g, 52.5 g, 53 g, 53.5 g, 54 g, 54.5 g, 55 g, 55.5 g, 56 g, 56.5 g, 57 g, 57.5 g, 58 g, 58.5 g, 59 g, 59.5 g, 60 g, 60.5 g, 61 g, 61.5 g, 62 g, 62.5 g, 63 g, 63.5 g, 64 g, 64.5 g, 65 g, 65.5 g, 66 g, 66.5 g, 67 g, 67.5 g, 68 g, 68.5 g, 69 g, 69.5 g, 70 g, 70.5 g, 71 g, 71.5 g, 72 g, 72.5 g, 73 g, 73.5 g, 74 g, 74.5 g, 75 g, 75.5 g, 76 g, 76.5 g, 77 g, 77.5 g, 78 g, 78.5 g, 79 g, 79.5 g, 80 g, 80.5 g, 81 g, 81.5 g, 82 g, 82.5 g, 83 g, 83.5 g, 84 g, 84.5 g, 85 g, 85.5 g, 86 g, 86.5 g, 87 g, 87.5 g, 88 g, 88.5 g, 89 g, 89.5 g, 90 g, 90.5 g, 91 g, 91.5 g, 92 g, 92.5 g, 93 g, 93.5 g, 94 g, 94.5 g, 95 g, 95.5 g, 96 g, 96.5 g, 97 g, 97.5 g, 98 g, 98.5 g, 99 g, 99.5 g, or about 100 g, or any dosage therebetween.

In some embodiments, a dosage of the antagonist of IGF1R signaling may be administered one, two, three, or four times daily for a time course of one day to several days, weeks, months, and even years, and may even be for the life of the patient. In some embodiments, a dosing regimen disclosed herein may last a period of at least about 30 minutes (min), one hour, 90 minutes, two hours, four hours, 8 hours, 16 hours, a day, a week, two weeks, three weeks, four weeks, from about 1 to about 4 weeks, from about 1 to about 8 weeks, from about 1 to about 12 weeks, from about 1 to about 16 weeks, from about 1 to about 20 weeks, from about 1 to about 24 weeks, from about 1 to about 36 weeks, from about 1 to about 48 weeks, from about 1 to about 52 weeks, from about 1 to about 60 weeks, from about 1 to about 72 weeks, from about 1 to about 84 weeks, from about 1 to about 96 weeks, from about 1 week to about 1 year, from about 1 week to about 2 years, from about 1 week to about 3 years, from 1 about week to about 4 years, from about 1 week to about 5 years, or longer. In some embodiments, a dosage of the antagonist of IGF1R signaling may be administered every day, every 2 days, every 3 days, every 4 days, every 5 days, every 6 days, every 7 days, every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, every 20 weeks, every 21 weeks, every 22 weeks, every 23 weeks, or every 24 weeks. In some embodiments, a dosage of the antagonist of IGF1R signaling may be administered over a course of about 5 seconds (s), 10 s, 20 s, 30 s, 40 s, 50 s, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or about 24 hours, or any duration therebetween.

In some embodiments, a dosing regimen of the antagonist of IGF1R signaling may last about 1, 2, 3, 4, 5, 6, or 7 days. In some embodiments, a dosing regimen of the antagonist of IGF1R signaling may last about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, 151 weeks, 152 weeks, 153 weeks, 154 weeks, 155 weeks, 156 weeks, 157 weeks, 158 weeks, 159 weeks, 160 weeks, 161 weeks, 162 weeks, 163 weeks, 164 weeks, 165 weeks, 166 weeks, 167 weeks, 168 weeks, 169 weeks, 170 weeks, 171 weeks, 172 weeks, 173 weeks, 174 weeks, 175 weeks, 176 weeks, 177 weeks, 178 weeks, 179 weeks, 180 weeks, 181 weeks, 182 weeks, 183 weeks, 184 weeks, 185 weeks, 186 weeks, 187 weeks, 188 weeks, 189 weeks, 190 weeks, 191 weeks, 192 weeks, 193 weeks, 194 weeks, 195 weeks, 196 weeks, 197 weeks, 198 weeks, 199 weeks, 200 weeks, 201 weeks, 202 weeks, 203 weeks, 204 weeks, 205 weeks, 206 weeks, 207 weeks, 208 weeks, 209 weeks, 210 weeks, 211 weeks, 212 weeks, 213 weeks, 214 weeks, 215 weeks, 216 weeks, 217 weeks, 218 weeks, 219 weeks, 220 weeks, 221 weeks, 222 weeks, 223 weeks, 224 weeks, 225 weeks, 226 weeks, 227 weeks, 228 weeks, 229 weeks, 230 weeks, 231 weeks, 232 weeks, 233 weeks, 234 weeks, 235 weeks, 236 weeks, 237 weeks, 238 weeks, 239 weeks, 240 weeks, 241 weeks, 242 weeks, 243 weeks, 244 weeks, 245 weeks, 246 weeks, 247 weeks, 248 weeks, 249 weeks, 250 weeks, 251 weeks, 252 weeks, 253 weeks, 254 weeks, 255 weeks, 256 weeks, 257 weeks, 258 weeks, 259 weeks, 260 weeks, 261 weeks, 262 weeks, 263 weeks, 264 weeks, 265 weeks, 266 weeks, 267 weeks, 268 weeks, 269 weeks, 270 weeks, 271 weeks, 272 weeks, 273 weeks, 274 weeks, 275 weeks, 276 weeks, 277 weeks, 278 weeks, 279 weeks, 280 weeks, 281 weeks, 282 weeks, 283 weeks, 284 weeks, 285 weeks, 286 weeks, 287 weeks, 288 weeks, 289 weeks, 290 weeks, 291 weeks, 292 weeks, 293 weeks, 294 weeks, 295 weeks, 296 weeks, 297 weeks, 298 weeks, 299 weeks, 300 weeks, 301 weeks, 302 weeks, 303 weeks, 304 weeks, 305 weeks, 306 weeks, 307 weeks, 308 weeks, 309 weeks, or about 310 weeks, or any duration longer than about 310 weeks. In some embodiments, a dosing regimen of the antagonist of IGF1R signaling disclosed herein may last about 1 year, 2 years, 3 years, 4 years, 5 years, 6 years, 7 years, 8 years, 9 years, about 10 years, or any period of time longer than 10 years, or throughout the life of the individual being treated.

In some embodiments, teprotumumab may be administered at a dosage based on the weight of the individual. In some embodiments, teprotumumab may be administered at a dosage of about 0.01 to 500 milligrams per kilogram (mg/kg) of the individual's weight, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.9 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 30.5 mg/kg, 31 mg/kg, 31.5 mg/kg, 32 mg/kg, 32.5 mg/kg, 33 mg/kg, 33.5 mg/kg, 34 mg/kg, 34.5 mg/kg, 35 mg/kg, 35.5 mg/kg, 36 mg/kg, 36.5 mg/kg, 37 mg/kg, 37.5 mg/kg, 38 mg/kg, 38.5 mg/kg, 39 mg/kg, 39.5 mg/kg, 40 mg/kg, 40.5 mg/kg, 41 mg/kg, 41.5 mg/kg, 42 mg/kg, 42.5 mg/kg, 43 mg/kg, 43.5 mg/kg, 44 mg/kg, 44.5 mg/kg, 45 mg/kg, 45.5 mg/kg, 46 mg/kg, 46.5 mg/kg, 47 mg/kg, 47.5 mg/kg, 48 mg/kg, 48.5 mg/kg, 49 mg/kg, 49.5 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 255 mg/kg, 260 mg/kg, 265 mg/kg, 270 mg/kg, 275 mg/kg, 280 mg/kg, 285 mg/kg, 290 mg/kg, 295 mg/kg, 300 mg/kg, 305 mg/kg, 310 mg/kg, 315 mg/kg, 320 mg/kg, 325 mg/kg, 330 mg/kg, 335 mg/kg, 340 mg/kg, 345 mg/kg, 350 mg/kg, 355 mg/kg, 360 mg/kg, 365 mg/kg, 370 mg/kg, 375 mg/kg, 380 mg/kg, 385 mg/kg, 390 mg/kg, 395 mg/kg, 400 mg/kg, 405 mg/kg, 410 mg/kg, 415 mg/kg, 420 mg/kg, 425 mg/kg, 430 mg/kg, 435 mg/kg, 440 mg/kg, 445 mg/kg, 450 mg/kg, 455 mg/kg, 460 mg/kg, 465 mg/kg, 470 mg/kg, 475 mg/kg, 480 mg/kg, 485 mg/kg, 490 mg/kg, 495 mg/kg, or about 500 mg/kg, or any amount therebetween.

In some embodiments, teprotumumab may be administered at a dosage of about 0.01 mg/kg to about 0.1 mg/kg, about 0.01 mg/kg to about 0.09 mg/kg, about 0.01 mg/kg to about 0.08 mg/kg, about 0.01 mg/kg to about 0.07 mg/kg, about 0.01 mg/kg to about 0.06 mg/kg, about 0.01 mg/kg to about 0.05 mg/kg, about 0.01 mg/kg to about 0.04 mg/kg, about 0.01 mg/kg to about 0.03 mg/kg, about 0.01 mg/kg to about 0.02 mg/kg, about 0.02 mg/kg to about 0.1 mg/kg, about 0.02 mg/kg to about 0.09 mg/kg, about 0.02 mg/kg to about 0.08 mg/kg, about 0.02 mg/kg to about 0.07 mg/kg, about 0.02 mg/kg to about 0.06 mg/kg, about 0.02 mg/kg to about 0.05 mg/kg, about 0.02 mg/kg to about 0.04 mg/kg, about 0.02 mg/kg to about 0.03 mg/kg, about 0.03 mg/kg to about 0.1 mg/kg, about 0.03 mg/kg to about 0.09 mg/kg, about 0.03 mg/kg to about 0.08 mg/kg, about 0.03 mg/kg to about 0.07 mg/kg, about 0.03 mg/kg to about 0.06 mg/kg, about 0.03 mg/kg to about 0.05 mg/kg, about 0.03 mg/kg to about 0.04 mg/kg, about 0.04 mg/kg to about 0.1 mg/kg, about 0.04 mg/kg to about 0.09 mg/kg, about 0.04 mg/kg to about 0.08 mg/kg, about 0.04 mg/kg to about 0.07 mg/kg, about 0.04 mg/kg to about 0.06 mg/kg, about 0.04 mg/kg to about 0.05 mg/kg, about 0.05 mg/kg to about 0.1 mg/kg, about 0.05 mg/kg to about 0.09 mg/kg, about 0.05 mg/kg to about 0.08 mg/kg, about 0.05 mg/kg to about 0.07 mg/kg, about 0.05 mg/kg to about 0.06 mg/kg, about 0.06 mg/kg to about 0.1 mg/kg, about 0.06 mg/kg to about 0.09 mg/kg, about 0.06 mg/kg to about 0.08 mg/kg, about 0.06 mg/kg to about 0.07 mg/kg, about 0.07 mg/kg to about 0.1 mg/kg, about 0.07 mg/kg to about 0.09 mg/kg, about 0.07 mg/kg to about 0.08 mg/kg, about 0.08 mg/kg to about 0.1 mg/kg, about 0.08 mg/kg to about 0.09 mg/kg, or about 0.09 mg/kg to about 0.1 mg/kg.

In some embodiments, teprotumumab may be administered at a dosage of about 0.1 mg/kg to about 1 mg/kg, about 0.1 mg/kg to about 0.9 mg/kg, about 0.1 mg/kg to about 0.8 mg/kg, about 0.1 mg/kg to about 0.7 mg/kg, about 0.1 mg/kg to about 0.6 mg/kg, about 0.1 mg/kg to about 0.5 mg/kg, about 0.1 mg/kg to about 0.4 mg/kg, about 0.1 mg/kg to about 0.3 mg/kg, about 0.1 mg/kg to about 0.2 mg/kg, about 0.2 mg/kg to about 1 mg/kg, about 0.2 mg/kg to about 0.9 mg/kg, about 0.2 mg/kg to about 0.8 mg/kg, about 0.2 mg/kg to about 0.7 mg/kg, about 0.2 mg/kg to about 0.6 mg/kg, about 0.2 mg/kg to about 0.5 mg/kg, about 0.2 mg/kg to about 0.4 mg/kg, about 0.2 mg/kg to about 0.3 mg/kg, about 0.3 mg/kg to about 1 mg/kg, about 0.3 mg/kg to about 0.9 mg/kg, about 0.3 mg/kg to about 0.8 mg/kg, about 0.3 mg/kg to about 0.7 mg/kg, about 0.3 mg/kg to about 0.6 mg/kg, about 0.3 mg/kg to about 0.5 mg/kg, about 0.3 mg/kg to about 0.4 mg/kg, about 0.4 mg/kg to about 1 mg/kg, about 0.4 mg/kg to about 0.9 mg/kg, about 0.4 mg/kg to about 0.8 mg/kg, about 0.4 mg/kg to about 0.7 mg/kg, about 0.4 mg/kg to about 0.6 mg/kg, about 0.4 mg/kg to about 0.5 mg/kg, about 0.5 mg/kg to about 1 mg/kg, about 0.5 mg/kg to about 0.9 mg/kg, about 0.5 mg/kg to about 0.8 mg/kg, about 0.5 mg/kg to about 0.7 mg/kg, about 0.5 mg/kg to about 0.6 mg/kg, about 0.6 mg/kg to about 1 mg/kg, about 0.6 mg/kg to about 0.9 mg/kg, about 0.6 mg/kg to about 0.8 mg/kg, about 0.6 mg/kg to about 0.7 mg/kg, about 0.7 mg/kg to about 1 mg/kg, about 0.7 mg/kg to about 0.9 mg/kg, about 0.7 mg/kg to about 0.8 mg/kg, about 0.8 mg/kg to about 1 mg/kg, about 0.8 mg/kg to about 0.9 mg/kg, about 0.8 mg/kg to about 1 mg/kg, about 0.8 mg/kg to about 0.9 mg/kg, or about 0.9 mg/kg to about 1 mg/kg.

In some embodiments, teprotumumab may be administered at a dosage of about 1 mg/kg to about 10 mg/kg, about 1 mg/kg to about 9 mg/kg, about 1 mg/kg to about 8 mg/kg, about 1 mg/kg to about 7 mg/kg, about 1 mg/kg to about 6 mg/kg, about 1 mg/kg to about 5 mg/kg, about 1 mg/kg to about 4 mg/kg, about 1 mg/kg to about 3 mg/kg, about 1 mg/kg to about 2 mg/kg, about 2 mg/kg to about 10 mg/kg, about 2 mg/kg to about 9 mg/kg, about 2 mg/kg to about 8 mg/kg, about 2 mg/kg to about 7 mg/kg, about 2 mg/kg to about 6 mg/kg, about 2 mg/kg to about 5 mg/kg, about 2 mg/kg to about 4 mg/kg, about 2 mg/kg to about 3 mg/kg, about 3 mg/kg to about 10 mg/kg, about 3 mg/kg to about 9 mg/kg, about 3 mg/kg to about 8 mg/kg, about 3 mg/kg to about 7 mg/kg, about 3 mg/kg to about 6 mg/kg, about 3 mg/kg to about 5 mg/kg, about 3 mg/kg to about 4 mg/kg, about 4 mg/kg to about 10 mg/kg, about 4 mg/kg to about 9 mg/kg, about 4 mg/kg to about 8 mg/kg, about 4 mg/kg to about 7 mg/kg, about 4 mg/kg to about 6 mg/kg, about 4 mg/kg to about 5 mg/kg, about 5 mg/kg to about 10 mg/kg, about 5 mg/kg to about 9 mg/kg, about 5 mg/kg to about 8 mg/kg, about 5 mg/kg to about 7 mg/kg, about 5 mg/kg to about 6 mg/kg, about 6 mg/kg to about 10 mg/kg, about 6 mg/kg to about 9 mg/kg, about 6 mg/kg to about 8 mg/kg, about 6 mg/kg to about 7 mg/kg, about 7 mg/kg to about 10 mg/kg, about 7 mg/kg to about 9 mg/kg, about 7 mg/kg to about 8 mg/kg, about 8 mg/kg to about mg/kg, about 8 mg/kg to about 9 mg/kg, or about 9 mg/kg to about 10 mg/kg.

In some embodiments, teprotumumab may be administered at a dosage of about 10 mg/kg to about 100 mg/kg, about 10 mg/kg to about 90 mg/kg, about 10 mg/kg to about 80 mg/kg, about 10 mg/kg to about 70 mg/kg, about 10 mg/kg to about 60 mg/kg, about 10 mg/kg to about 50 mg/kg, about 10 mg/kg to about 40 mg/kg, about 10 mg/kg to about 30 mg/kg, about 10 mg/kg to about 20 mg/kg, about 20 mg/kg to about 100 mg/kg, about 20 mg/kg to about 90 mg/kg, about 20 mg/kg to about 80 mg/kg, about 20 mg/kg to about 70 mg/kg, about 20 mg/kg to about 60 mg/kg, about 20 mg/kg to about 50 mg/kg, about 20 mg/kg to about 40 mg/kg, about 20 mg/kg to about 30 mg/kg, about 30 mg/kg to about 100 mg/kg, about 30 mg/kg to about 90 mg/kg, about 30 mg/kg to about 80 mg/kg, about 30 mg/kg to about 70 mg/kg, about 30 mg/kg to about 60 mg/kg, about 30 mg/kg to about 50 mg/kg, about 30 mg/kg to about 40 mg/kg, about 40 mg/kg to about 100 mg/kg, about 40 mg/kg to about 90 mg/kg, about 40 mg/kg to about 80 mg/kg, about 40 mg/kg to about 70 mg/kg, about 40 mg/kg to about 60 mg/kg, about 40 mg/kg to about 50 mg/kg, about 50 mg/kg to about 100 mg/kg, about 50 mg/kg to about 90 mg/kg, about 50 mg/kg to about 80 mg/kg, about 50 mg/kg to about 70 mg/kg, about 50 mg/kg to about 60 mg/kg, about 60 mg/kg to about 100 mg/kg, about 60 mg/kg to about 90 mg/kg, about 60 mg/kg to about 80 mg/kg, about 60 mg/kg to about 70 mg/kg, about 70 mg/kg to about 100 mg/kg, about 70 mg/kg to about 90 mg/kg, about 70 mg/kg to about 80 mg/kg, about 80 mg/kg to about 100 mg/kg, about 80 mg/kg to about 90 mg/kg, or about 90 mg/kg to about 100 mg/kg.

In some embodiments, a dosage of teprotumumab disclosed herein may be administered one, two, three, or four times daily for a time course of one day to several days, weeks, months, and even years, and may even be for the life of the patient. In some embodiments, a dosing regimen of teprotumumab disclosed herein may last a period of at least about 30 minutes, one hour, 90 minutes, two hours, four hours, 8 hours, 16 hours, a day, a week, two weeks, three weeks, four weeks, from about 1 to about 4 weeks, from about 1 to about 8 weeks, from about 1 to about 12 weeks, from about 1 to about 16 weeks, from about 1 to about 20 weeks, from about 1 to about 24 weeks, from about 1 to about 36 weeks, from about 1 to about 48 weeks, from about 1 to about 52 weeks, from about 1 to about 60 weeks, from about 1 to about 72 weeks, from about 1 to about 84 weeks, from about 1 to about 96 weeks, from about 1 week to about 1 year, from about 1 week to about 2 years, from about 1 week to about 3 years, from 1 about week to about 4 years, from about 1 week to about 5 years, or longer. In some embodiments, a dosage of teprotumumab disclosed herein may be administered every day, every 2 days, every 3 days, every 4 days, every 5 days, every 6 days, every 7 days, every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, every 20 weeks, every 21 weeks, every 22 weeks, every 23 weeks, or every 24 weeks. In some embodiments, a dosage of teprotumumab disclosed herein may be administered over a course of about 5 seconds (s), 10 s, 20 s, 30 s, 40 s, 50 s, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or about 24 hours, or any duration therebetween. In some embodiments, a dosage of teprotumumab disclosed herein may be administered over a course of about 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, or about 90 min, or any duration therebetween.

In some embodiments, a dosing regimen of teprotumumab disclosed herein may last about 1, 2, 3, 4, 5, 6, or 7 days. In some embodiments, a dosing regimen of teprotumumab disclosed herein may last about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, 151 weeks, 152 weeks, 153 weeks, 154 weeks, 155 weeks, 156 weeks, 157 weeks, 158 weeks, 159 weeks, 160 weeks, 161 weeks, 162 weeks, 163 weeks, 164 weeks, 165 weeks, 166 weeks, 167 weeks, 168 weeks, 169 weeks, 170 weeks, 171 weeks, 172 weeks, 173 weeks, 174 weeks, 175 weeks, 176 weeks, 177 weeks, 178 weeks, 179 weeks, 180 weeks, 181 weeks, 182 weeks, 183 weeks, 184 weeks, 185 weeks, 186 weeks, 187 weeks, 188 weeks, 189 weeks, 190 weeks, 191 weeks, 192 weeks, 193 weeks, 194 weeks, 195 weeks, 196 weeks, 197 weeks, 198 weeks, 199 weeks, 200 weeks, 201 weeks, 202 weeks, 203 weeks, 204 weeks, 205 weeks, 206 weeks, 207 weeks, 208 weeks, 209 weeks, 210 weeks, 211 weeks, 212 weeks, 213 weeks, 214 weeks, 215 weeks, 216 weeks, 217 weeks, 218 weeks, 219 weeks, 220 weeks, 221 weeks, 222 weeks, 223 weeks, 224 weeks, 225 weeks, 226 weeks, 227 weeks, 228 weeks, 229 weeks, 230 weeks, 231 weeks, 232 weeks, 233 weeks, 234 weeks, 235 weeks, 236 weeks, 237 weeks, 238 weeks, 239 weeks, 240 weeks, 241 weeks, 242 weeks, 243 weeks, 244 weeks, 245 weeks, 246 weeks, 247 weeks, 248 weeks, 249 weeks, 250 weeks, 251 weeks, 252 weeks, 253 weeks, 254 weeks, 255 weeks, 256 weeks, 257 weeks, 258 weeks, 259 weeks, 260 weeks, 261 weeks, 262 weeks, 263 weeks, 264 weeks, 265 weeks, 266 weeks, 267 weeks, 268 weeks, 269 weeks, 270 weeks, 271 weeks, 272 weeks, 273 weeks, 274 weeks, 275 weeks, 276 weeks, 277 weeks, 278 weeks, 279 weeks, 280 weeks, 281 weeks, 282 weeks, 283 weeks, 284 weeks, 285 weeks, 286 weeks, 287 weeks, 288 weeks, 289 weeks, 290 weeks, 291 weeks, 292 weeks, 293 weeks, 294 weeks, 295 weeks, 296 weeks, 297 weeks, 298 weeks, 299 weeks, 300 weeks, 301 weeks, 302 weeks, 303 weeks, 304 weeks, 305 weeks, 306 weeks, 307 weeks, 308 weeks, 309 weeks, or 310 weeks, or any period of time longer than 310 weeks. In some embodiments, a dosing regimen of teprotumumab disclosed herein may last about 1 year, 2 years, 3 years, 4 years, 5 years, 6 years, 7 years, 8 years, 9 years, 10 years, or any period of time longer than 10 years, or throughout the life of the individual being treated.

In some embodiments, teprotumumab may be administered at different doses throughout the treatment. In some embodiments, teprotumumab may be administered at a first dose and a second dose throughout the treatment. In some embodiments, the dosage of teprotumumab is calculated based on the weight of the individual. In some embodiments, teprotumumab may be administered at the first dose of about 10 mg/kg through intravenous infusion at the beginning of the treatment. In some embodiments, teprotumumab may be administered at the first dose of about 10 mg/kg over the course of about 60 min to about 90 min through intravenous infusion at the beginning of the treatment. In some embodiments, treatment with the first dose of teprotumumab may last a period of time disclosed herein. In some embodiments, treatment with the first dose of teprotumumab may last a period of time from about 1 week to about 30 weeks days. In some embodiments, treatment with the first dose of teprotumumab may last a period of time from about 1 week to about 10 weeks. In some embodiments, treatment with the first dose of teprotumumab may last a period of time from about 1 day to about 5 weeks. In some embodiments, treatment with the first dose of teprotumumab may last 3 weeks. In some embodiments, teprotumumab may be administered at the second dose of about 20 mg/kg through intravenous infusion after the initial treatment with the first dose. In some embodiments, teprotumumab may be administered at the second dose of about 20 mg/kg through intravenous infusion over the course of about 60 min to about 90 min after the initial treatment with the first dose. In some embodiments, teprotumumab may be administered at the second dose of about 20 mg/kg through intravenous infusion over the course of about 60 min to about 90 min 3 weeks after the initial treatment with the first dose. In some embodiments, teprotumumab may be administered at about 20 mg/kg through intravenous infusion once every 3 weeks. In some embodiments, treatment with the second dose of teprotumumab may last a period of time disclosed herein. In some embodiments, treatment with the second dose of teprotumumab may last a period of time from about 1 week to about 200 weeks. In some embodiments, treatment with the second dose of teprotumumab may last a period of time from about 1 week to about 100 weeks. In some embodiments, treatment with the second dose of teprotumumab may last a period of time from about 1 week to about 50 weeks. In some embodiments, treatment with the second dose of teprotumumab may last a period of time from about 1 week to about 25 weeks. In some embodiments, treatment with the second dose of teprotumumab may last a period of time of about 21 weeks. In some embodiments, teprotumumab may be administered at about 20 mg/kg through intravenous infusion every 3 weeks for about 7 additional infusions. In some embodiments, the initial dose of teprotumumab may be adjusted based on the factors disclosed herein. In some embodiments, the dose of teprotumumab after initial treatment may be adjusted based on the factors disclosed herein.

In some embodiments, an antagonist of IGF1R signaling disclosed herein or a pharmaceutical composition comprising an antagonist of IGF1R signaling disclosed herein may be in various forms, including but not limited to lyophilized powder, a solution, a tablets, a pill, a sachet, or a capsule of hard or soft gelatin, methylcellulose or of another suitable material easily dissolved in the digestive tract. In some embodiments, the excipient of the pharmaceutical composition may comprise any suitable substance, including but not limited pharmaceutical grades of mannitol, lactose, starch, magnesium stearate, sodium saccharin, talcum, cellulose, glucose, sucrose, magnesium carbonate, and the like.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Individuals Treated with Teprotumumab have Increased Risk of Hyperglycemia if Previously Exhibited Poor Glucose Control

In a trial of adverse events associated with individuals receiving teprotumumab. Subjects reporting hyperglycemia were stratified by pre-existing diabetes as indicated in Table 1.

TABLE 1
Subjects with an AESI of Hyperglycemia During the 24-Week
Treatment Period of the TED RCTs
Stratified by History of Pre-existing Diabetes

| | Double-Masked Population (RCTs) | | | |
|---|---|---|---|---|
| | Placebo | | Teprotumumab | |
| | N = 86 | | N = 84 | |
| MedDRA PT | n (%) | | n (%) | |
| Pre-existing diabetes or glucose | Yes | No | Yes | No |
| intolerance, n (%) | (n = 9) | (n = 77) | (n = 10) | (n = 74) |
| Any subject with hyperglycemia | 0 | 1 (1.3) | 5 (50) | 3 (4.1) |
| Hyperglycemia | 0 | 1 (1.3) | 5 (50) | 1 (1.4) |
| Blood glucose increased | 0 | 0 | 0 | 2 (2.7) |

Overall, 10 subjects in the teprotumumab group had pre-existing diabetes. Of these, 5 subjects (50%) reported hyperglycemia and one subject, who did not report hyperglycemia, had an increase in HbA1c from 7.4% on Day −45 to 8.2% on Day 85 that subsequently decreased to 7.8% on Day 169. Of the 74 subjects in the teprotumumab group without pre-existing diabetes, 3 subjects (4.1%) reported an event of hyperglycemia during the Treatment Period. Of the 9 subjects in the placebo group with pre-existing diabetes mellitus, none reported hyperglycemia and of the 77 subjects in the placebo group without pre-existing diabetes, 1 subject (1.3%) reported hyperglycemia. Thus, individuals with pre-existing diabetes or poor glucose control were more likely to develop an adverse event of hyperglycemia.

### Example 2. Treatment of Thyroid Eye Disease with Teprotumumab in Individual No. 1

This example illustrates a method of treating an individual with thyroid eye disease using teprotumumab, an antibody that binds IGF1R. First, the individual with thyroid eye disease is subjected to a test for blood glucose control. The test for blood glucose control is a measurement of hemoglobin A1c (HbA1c) or can be any other suitable test for blood glucose control.

When the measurement of HbA1c is below 6.5%, the individual is selected for treatment with teprotumumab. Teprotumumab is administered through intravenous infusion over a course of 90 min at a dose of 10 mg/kg based on the weight of the individual. For example, when the individual weighs 75 kg, an amount of 750 mg of teprotumumab is administered. Then, in 3 weeks, the individual is administered a second dose of 20 mg/kg of teprotumumab through intravenous infusion over the course of 90 min based on the weight of the individual. For example, when the individual weighs 75 kg, an amount of 1.5 g of teprotumumab is administered. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, by the measurement of HbA1c.

During the teprotumumab treatment, when the measurement of HbA1c is 6.5% or higher, the treatment of teprotumumab is discontinued. The individual is then subjected to a treatment for high blood glucose, such as insulin. During the treatment for high blood glucose, the individual is subjected to regular test for blood glucose control. When the measurement of HbA1c is below 6.5% during the treatment for high blood glucose, the treatment of teprotumumab is renewed. The individual's blood glucose level is monitored continuously throughout the teprotumumab treatment.

### Example 3. Treatment of Thyroid Eye Disease with Teprotumumab in Individual No. 2

This example illustrates a method of treating another individual with thyroid eye disease using teprotumumab. First, the individual with thyroid eye disease is subjected to a test for blood glucose control. The test for blood glucose control is a measurement of HbA1c or can be any other suitable test for blood glucose control.

When the measurement of HbA1c is below 6.5%, the individual is selected for treatment with teprotumumab. Teprotumumab is administered through intravenous infusion over a course of 90 min at a dose of 10 mg/kg based on the weight of the individual. Then, in 3 weeks, the individual is administered a second dose of 20 mg/kg of teprotumumab through intravenous infusion over the course of 90 min based on the weight of the individual. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, using the measurement of HbA1c.

During the course of the teprotumumab treatment, when the measurement of HbA1c is 6.5% or higher, the teprotumumab treatment is continued at a dose lower than 20 mg/kg and adjusted from time to time based on the blood glucose level of the individual by the measurement of HbA1c.

### Example 4. Treatment of Thyroid Eye Disease with Teprotumumab in Individual No. 3

This example illustrates a method of treating another individual with thyroid eye disease using teprotumumab. First, the individual with thyroid eye disease is subjected to a test for blood glucose control. The test for blood glucose control is a measurement of HbA1c or can be any other suitable test for blood glucose control.

When the measurement of HbA1c is below 6.5%, the individual is selected for treatment with teprotumumab. Teprotumumab is administered through intravenous infusion over a course of 90 min at a dose of 10 mg/kg based on the weight of the individual. Then, in 3 weeks, the individual is administered a second dose of 20 mg/kg of teprotumumab through intravenous infusion over the course of 90 min based on the weight of the individual. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, using the measurement of HbA1c.

During the course of the teprotumumab treatment, when the measurement of HbA1c is 6.5% or higher, the teprotumumab treatment is terminated.

### Example 5. Treatment of Thyroid Eye Disease with Teprotumumab in Individual No. 4

This example illustrates a method of treating another individual with thyroid eye disease using teprotumumab. First, the individual with thyroid eye disease is subjected to a test for blood glucose control. The test for blood glucose control is a measurement of HbA1c or can be any other suitable test for blood glucose control.

When the individual's measurement of HbA1c is 6.5% or higher, the individual is subjected to a treatment for high blood glucose, such as insulin. Throughout the treatment for high blood glucose, the individual is regularly tested for blood glucose level by a measurement of HbA1c. When the measurement of HbA1c is below 6.5% during the course of the treatment for high blood glucose, teprotumumab is administered through intravenous infusion over a course of 90 min at a dose of 10 mg/kg based on the weight of the individual. Then, in 3 weeks, the individual is administered a second dose of 20 mg/kg of teprotumumab through intravenous infusion over the course of 90 min based on the weight of the individual. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, using the measurement of HbA1c.

During the teprotumumab treatment, when the measurement of HbA1c is 6.5% or higher, the treatment of teprotumumab is discontinued. The individual is then subjected to a treatment for high blood glucose again, such as insulin. During the treatment for high blood glucose, the individual is subjected to regular test for blood glucose control. When the measurement of HbA1c is below 6.5% during the treatment for high blood glucose, the treatment of teprotumumab is renewed. The individual's blood glucose level is monitored continuously throughout the teprotumumab treatment.

### Example 6. Treatment of Thyroid Eye Disease with Teprotumumab in Individual No. 5

This example illustrates a method of treating another individual with thyroid eye disease using teprotumumab. First, the individual with thyroid eye disease is subjected to a test for blood glucose control. The test for blood glucose control is a measurement of HbA1c or can be any other suitable test for blood glucose control.

When the individual's measurement of HbA1c is 6.5% or higher, the individual is subjected to a treatment for high blood glucose, such as insulin. Throughout the treatment for high blood glucose, the individual is regularly tested for blood glucose level by a measurement of HbA1c. When the measurement of HbA1c is below 6.5% during the course of the treatment for high blood glucose, teprotumumab is administered through intravenous infusion over a course of 90 min at a dose of 10 mg/kg based on the weight of the individual. Then, in 3 weeks, the individual is administered a second dose of 20 mg/kg of teprotumumab through intravenous infusion over the course of 90 min based on the weight of the individual. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, using the measurement of HbA1c.

During the teprotumumab treatment, when the measurement of HbA1c is 6.5% or higher, the teprotumumab treatment is continued at a dose lower than 20 mg/kg and adjusted from time to time based on the blood glucose level of the individual by the measurement of HbA1c.

### Example 7. Treatment of Thyroid Eye Disease with Teprotumumab in Individual No. 6

This example illustrates a method of treating another individual with thyroid eye disease using teprotumumab. First, the individual with thyroid eye disease is subjected to a test for blood glucose control. The test for blood glucose control is a measurement of HbA1c or can be any other suitable test for blood glucose control.

When the individual's measurement of HbA1c is 6.5% or higher, the individual is subjected to a treatment for high blood glucose, such as insulin. Throughout the treatment for high blood glucose, the individual is regularly tested for blood glucose level by a measurement of HbA1c. When the measurement of HbA1c is below 6.5% during the course of the treatment for high blood glucose, teprotumumab is administered through intravenous infusion over a course of 90 min at a dose of 10 mg/kg based on the weight of the individual. Then, in 3 weeks, the individual is administered a second dose of 20 mg/kg of teprotumumab through intravenous infusion over the course of 90 min based on the weight of the individual. 20 mg/kg of teprotumumab is administered to the individual once every 3 weeks for a total of 7 times. Throughout the course of the teprotumumab treatment, the effects of teprotumumab on thyroid eye disease are monitored periodically, such as once every week, and the effects of teprotumumab on blood glucose level are monitored periodically, such as once every day, using the measurement of HbA1c.

During the teprotumumab treatment, when the measurement of HbA1c is 6.5% or higher, the teprotumumab treatment is terminated.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-207. (canceled)

208. A method of treating an individual afflicted with an insulin-like growth factor 1 receptor (IGF1R) associated disorder the method comprising:
a) obtaining the results of a test for blood glucose control from the individual; and
b) administering an antagonist of IGF1R signaling to the individual if the individual exhibits normal blood glucose control by the test for blood glucose control.

209. The method of claim 208, wherein the IGF1R associated disorder comprises thyroid eye disease.

210. The method of claim 208, wherein the antagonist of IGF1R signaling comprises an antibody that binds IGF1R.

211. The method of claim 210, wherein the antibody that binds IGF1R comprises ganitumab, figitumumab, MEDI-573, cixutumumab, dalotuzumab, robatumumab, AVE1642, BIIB022, xentuzumab, istiratumab, or linsitinib.

212. The method of claim 210, wherein the antibody that binds IGF1R comprises teprotumumab.

213. The method of claim 208, wherein the antagonist of IGF1R signaling comprises a small molecule that inhibits IGF1R signaling.

214. The method of claim 213, wherein the small molecule that inhibits IGF1R signaling comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, INSM-18, AZD3463, AZD9362, BI885578, BI893923, TT-100 (NDGA), XL-228, ganetespib, ceritinib, or A-928605.

215. The method of claim 210, wherein the antibody that binds IGF1R comprises:
a) a heavy chain comprising:
(i) a first heavy chain complementarity determining region (HCDR1) comprising an amino acid sequence according to SEQ ID No.: 5;
(ii) a second heavy chain complementarity determining region (HCDR2) comprising an amino acid sequence according to SEQ ID No.: 6; and
(iii) a third heavy chain complementarity determining region (HCDR3) comprising an amino acid sequence according to SEQ ID No.: 7; and
b) a light chain comprising:
(i) a first light chain complementarity determining region (LCDR1) comprising an amino acid sequence according to SEQ ID No.: 8;
(ii) a second light chain complementarity determining region (LCDR2) comprising an amino acid sequence according to SEQ ID No.: 9; and
(iii) a third light chain complementarity determining region (LCDR3) comprising an amino acid sequence according to SEQ ID No.: 10.

216. The method of claim 210, wherein the antibody that binds IGF1R comprises:
a) a heavy chain variable domain comprising an amino acid sequence having at least 85%, 90%, or 95% sequence identity to SEQ ID No.: 3; and
b) a light chain variable domain comprising an amino acid sequence having at least 85%, 90%, or 95% sequence identity to SEQ ID No.: 4.

217. The method of claim 210, wherein the antibody that binds IGF1R comprises:
a) a heavy chain variable domain comprising an amino acid sequence according to SEQ ID No.: 3; and
b) a light chain variable domain comprising an amino acid sequence according to SEQ ID No.: 4.

218. The method of claim 210, wherein the antibody that binds IGF1R comprises:
a) a heavy chain comprising an amino acid sequence having at least 85%, 90%, or 95% sequence identity to SEQ ID No.: 1; and
b) a light chain comprising an amino acid sequence having at least 85%, 90%, or 95% sequence identity to SEQ ID No.: 2.

219. The method of claim 210, wherein the antibody that binds IGF1R comprises:
a) a heavy chain comprising an amino acid sequence according to SEQ ID No.: 1; and
b) a light chain comprising an amino acid sequence according to SEQ ID No.: 2.

220. The method of claim 208, wherein the test for blood glucose control comprises a measurement of hemoglobin A1c (HbA1c).

221. The method of claim 220, wherein the individual exhibits normal blood glucose control if the measurement of HbA1c is below about 6.5%.

222. The method of claim 208, wherein the test for blood glucose control comprises an oral glucose tolerance test.

223. The method of claim 222, wherein the individual exhibits normal blood glucose control if the individual exhibits a blood glucose level below about 200 mg/dL about two hours after oral ingestion of about 75 grams of sugar.

224. The method of claim 208, wherein the test for blood glucose control comprises a measurement of fasting blood glucose.

225. The method of claim 224, wherein the individual exhibits normal blood glucose control if the individual exhibits a blood glucose level below about 126 mg/dL after an overnight fast.

226. The method of claim 225, wherein the overnight fast lasts at least about 8 hours.

227. The method of claim 208, wherein the test for blood glucose control comprises a measurement of non-fasting blood glucose.

228. The method of claim 227, wherein the individual exhibits normal blood glucose control if the individual exhibits a non-fasting blood glucose level below about 200 mg/dL.

229. The method of claim 208, wherein the individual is diagnosed with hyperglycemia, prediabetes, or diabetes before the treatment with the antagonist of IGF1R signaling.

230. The method of claim 208, wherein the administering comprises intravenous infusion.

231. The method of claim 208, wherein the administering comprises administering about 10 mg/kg of the antagonist of IGF1R signaling over the course of about 60 min to about 90 min based on the weight of the individual.

232. The method of claim 231, wherein the administering further comprises administering about 20 mg/kg of the antagonist of IGF1R signaling over the course of about 60 min to about 90 min based on the weight of the individual.

233. The method of claim 232, wherein the administering further comprises administering about 20 mg/kg of the antagonist of IGF1R signaling once every 3 weeks for about 7 times.

234. A method of treating an individual afflicted with an insulin-like growth factor 1 receptor (IGF1R) associated disorder, the method comprising:
a) selecting an individual afflicted with the IGF1R associated disorder for treatment with an antagonist of IGF1R signaling if the individual exhibits normal blood glucose control by a first test for blood glucose control;
b) administering a first dose of the antagonist of IGF1R signaling to the individual; and
c) obtaining the results of a second test for blood glucose control from the individual being treated with the antagonist of IGF1R signaling.

235. The method of claim 234, further comprising interrupting treatment of the individual with the antagonist of IGF1R signaling if the individual exhibits abnormal blood glucose control by the results of the second test for blood glucose control.

236. The method of claim 234, further comprising administering a second dose of the antagonist of IGF1R signaling to the individual if the individual exhibits abnormal blood glucose control by the results of the second test for blood glucose control, wherein the second dose is lower than the first dose.

237. A method of treating an individual afflicted with an insulin-like growth factor 1 receptor (IGF1R) associated disorder wherein the individual is also afflicted with diabetes, the method comprising:
a. administering one or more anti-diabetic drugs to lower blood glucose levels; and
b. administering a dose of the antagonist of IGF1R signaling to the individual.
